(19) United States
(12) Patent Application Publication
(10) Pub. No.: US 2009/0143423 A1
(43) Pub. Date: Jun. 4, 2009

(54) SOLID DISPERSION PRODUCT CONTAINING N-ARYL UREA-BASED COMPOUND

(75) Inventors: Rudolf Schroeder, Worms (DE); Tanja Heitermann, Stutensee (DE)

Correspondence Address: EDWARDS ANGELL PALMER & DODGE LLP, P.O. BOX 55874, BOSTON, MA, 02205, US

(73) Assignee: ABBOTT GMBH & CO. KG, Wiesbaden (DE)

(21) Appl. No.: 12/253,499

(22) Filed: Oct. 17, 2008

Related U.S. Application Data
(60) Provisional application No. 60/999,613, filed on Oct. 19, 2007.

(51) Int. Cl. A61K 31/17; A61K 31/416; A61K 31/454; A61K 31/4725

(52) U.S. Cl. 514/307; 514/406; 514/596; 514/338

(57) ABSTRACT

A solid dispersion product comprising at least one N-aryl urea-based pharmaceutically active agent or an agent of related structural type is obtained by a) preparing a liquid mixture containing the at least one active agent, at least one pharmaceutically acceptable matrix-forming agent, at least one pharmaceutically acceptable surfactant and at least one solvent, and b) removing the solvent(s) from the liquid mixture to obtain the solid dispersion product.

### RELATED APPLICATION DATA

This application claims priority to U.S. provisional application Ser. No. 60/999,613, filed Oct. 19, 2007, the entire disclosure of which is incorporated herein by this reference.

Many potent drugs belong to the class of compounds of N-aryl ureas or compounds of related structural types. Unfortunately, the crystalline forms of most N-aryl urea-based active agents or compounds of related structural types are characterized by poor solubility in aqueous liquids.

Drugs of low water solubility, for example those classified as “practically insoluble” or “insoluble” according to United States Pharmacopeia (USP) 24 (2000), p. 10, i.e., having a solubility of less than about 1 part per 10,000 parts water (less than about 100 μg/ml) are notoriously difficult to formulate for oral delivery. Among other problems, bioavailability of such drugs, when administered by the oral route, tends to be very low.

A specific illustrative small-molecule drug of low water solubility is the compound 1-((R)-5-tert-butyl-indan-1-yl)-3-(1H-indazol-4-yl)-urea (ABT-102), a first-in-class TRPV1 antagonist, intended for the treatment of pain. ABT-102 has a molecular weight of 348.44 g/mol and is disclosed in U.S. Pat. No. 7,015,233 and WO 2004/111009.

For a variety of reasons, such as patient compliance and taste masking, a solid dosage form is usually preferred over a liquid dosage form. In most instances, however, oral solid dosage forms of a drug provide a lower bioavailability than oral solutions of the drug.

There remains a need in the pharmaceutical art for a novel solid formulation of active agents of low water solubility such as ABT-102 that is suitable for oral administration. More particularly and without limitation, there is a need for such a formulation having at least one of the following features, advantages or benefits: acceptably high concentration of the drug; and acceptable bioavailability when administered orally.

### SUMMARY OF THE INVENTION

In one aspect, the invention provides a solid dispersion product comprising at least one pharmaceutically active agent, obtained by
a) preparing a liquid mixture containing the at least one active agent, at least one pharmaceutically acceptable matrix-forming agent, at least one pharmaceutically acceptable surfactant and at least one solvent, and
b) removing the solvent(s) from the liquid mixture to obtain the solid dispersion product.

In another aspect, the invention provides a pharmaceutical dosage form comprising a solid dispersion product comprising at least one pharmaceutically active agent, obtained by
a) preparing a liquid mixture containing the at least one active agent, at least one pharmaceutically acceptable matrix-forming agent, at least one pharmaceutically acceptable surfactant and at least one solvent, and
b) removing the solvent(s) from the liquid mixture to obtain the solid dispersion product.

In yet another aspect, the invention provides a process for preparing a solid dispersion product comprising at least one pharmaceutically active agent, which process comprises
a) preparing a liquid mixture containing the at least one active agent, at least one pharmaceutically acceptable matrix-forming agent, at least one pharmaceutically acceptable surfactant and at least one solvent, and
b) removing the solvent(s) from the liquid mixture to obtain the solid dispersion product.

### BRIEF DESCRIPTION OF THE DRAWINGS

FIG. 1 shows PXRD patterns of an excipient mixture containing Kollidon-30, Gelucire 44/14, and Vitamin E-TPGS (FIG. 1, top) and of crystalline ABT-102 (FIG. 1, bottom).

FIG. 2 shows PXRD patterns of the spray-dried solid dispersions after being stored at 40° C./75% RH for 4 weeks (top two, with 15% drug load) and 6 weeks (bottom four, with 25% drug load).

### DETAILED DESCRIPTION OF THE INVENTION

The invention is particularly useful for water-insoluble or poorly water-soluble (or “hydrophobic” or “lipophilic”) compounds. Compounds are considered water-insoluble or poorly water-soluble when their solubility in water at 25° C. is less than 1 g/100 ml, especially less than 0,1 g/100 ml.

In the dosage forms of the invention, the active agent is present as a solid dispersion or, preferably, as a solid solution. The term “solid dispersion” defines a system in a solid state (as opposed to a liquid or gaseous state) comprising at least two components, wherein one component is dispersed evenly throughout the other component or components. For example, the active agent or combination of active agents is dispersed in a matrix comprised of the matrix-forming agent(s) and pharmaceutically acceptable surfactant(s). The term “solid dispersion” encompasses systems having small particles, typically of less than 1 μm in diameter, of one phase dispersed in another phase. When said dispersion of the components is such that the system is chemically and physically uniform or homogenous throughout or consists of one phase (as defined in thermodynamics), such a solid dispersion will be called a “solid solution” or a “glassy solution”. A glassy solution is a homogeneous, glassy system in which a solute is dissolved in a glassy solvent. Glassy solutions and solid solutions are preferred physical systems. These systems do not contain any significant amounts of active agents in their crystalline or microcrystalline state, as evidenced by thermal analysis (DSC) or X-ray diffraction analysis (WAXS).

In an embodiment of the invention, at least one filler is added to the liquid mixture before removing the solvent(s). It was found that incorporation of a filler into the liquid mixture before removing the solvent(s) increases the brittleness of the solid dispersion product obtained. This allows the solid dispersion product to be subjected to a direct tabletting process.

Preferably, the filler is essentially insoluble in the liquid mixture.

The choice of fillers is not particularly restricted. The filler may be suitably selected from inorganic particulate materials such as silica, calcium carbonate, calcium phosphates, titanium dioxide; natural and pre-gelatinized starches such as corn starch, cereal starch, potato starch; or the like.

However, the filler is preferably water-soluble. Useful fillers to that end may be selected from sugars such as lactose, sucrose; sugar alcohols such as mannitol, sorbitol, xylitol; or sugar alcohol derivatives.

The relative amounts of active agent, pharmaceutically acceptable matrix-forming agent and pharmaceutically acceptable surfactant are chosen with the following conditions in mind: (1) Essentially all of the active agent should be dispersed evenly throughout the matrix comprised of the matrix-forming agent(s) and pharmaceutically acceptable surfactant(s). (2) The matrix should have sufficient mechanical integrity and stability; in particular, the matrix should not exhibit cold flow. Generally, the mass ratio of active agent and pharmaceutically acceptable matrix-forming agent is from 0.01:1 to 1:3, preferably 0.05:1 to 0.2:1; generally the mass ratio of active agent and pharmaceutically acceptable surfactant(s) is from 0.1:1 to 1:7, preferably 1:4 to 1:6.5.

Generally, the solid dispersion product comprises

from about 1 to 30% by weight, preferably from about 4 to 15% by weight, of said at least one pharmaceutically active agent,
from about 15 to 70% by weight, preferably from about 20 to 55% by weight, of said at least one pharmaceutically acceptable matrix-forming agent,
from about 2 to 70% by weight, preferably from about 5 to 55% by weight, of said at least one surfactant, and
from about 0 to 80% by weight, preferably from about 0 to 60% by weight, of additives such as fillers.

The matrix-forming agent may be any agent capable of embedding an active agent and/or being loaded with an active agent and stabilizing an essentially amorphous state of the active agent. Mixtures of matrix-forming agents can, of course, be used.

The pharmaceutically acceptable matrix-forming agent is suitably selected from the group consisting of cyclodextrines, pharmaceutically acceptable polymers, lipids or combinations of two or more thereof.

Cyclodextrins for the purpose of the invention are cyclic oligo- or polysaccharides, for example so-called cycloamyloses or cycloglucans, and analogous cyclic carbohydrates which are described, for example, in Angew. Chem. 92 (1980) p. 343 or F. Vögtle, Supramolekulare Chemie, 2nd Edition, (1992). Suitable and preferred are those cyclodextrins which have a structure suitable for interactions with active agent molecules, in particular in the sense of host-guest systems. Particularly suitable cyclodextrins are those consisting of 6, 7, 8 or 9 α-1,4-glycosidically linked glucose units, which are called (α-, β-, γ- or δ-cyclodextrins. Higher structures analogous to cyclodextrins and composed of a larger number of glucoses or similar sugars are also conceivable and suitable.

Also suitable as cyclodextrins are modified cyclodextrins such as, for example, products which can be prepared by reacting cyclodextrins with alkylene oxides, alkyl halides, acid chlorides, epihalohydrins, isocyanates or halogenated carboxylic acids. Thus, suitable examples are products of the reaction of cyclodextrins with alkylene oxides such as ethylene oxide, propylene oxide, butylene oxide or styrene oxide. One, more than one or all hydroxyl groups in the cyclodextrin polyethers formed in this way may be substituted. Depending on the degree of substitution or the chain lengths of the polyether units, the average molar degree of substitution, that is to say the number of moles of alkylene oxide with which one mole of cyclodextrin is reacted, is usually between 3 and 20,000, but there is in principle no upper limit. Particularly suitable examples are the products of the reaction of cyclodextrins with alkylating agents such as C₁-C₂₂-alkyl halides, for example methyl chloride, ethyl chloride, isopropyl chloride, n-butyl chloride, isobutyl chloride, benzyl chloride, lauryl chloride, stearyl chloride, methyl bromide, ethyl bromide, n-butyl bromide and dialkyl sulfates such as, for example, dimethyl sulfate or diethyl sulfate. Reaction with alkylating reagents leads to cyclodextrin ethers in which one, more than one or all hydroxyl groups are substituted by alkyl ether groups. With the cyclodextrins composed of glucose units, the average degree of etherification per glucose unit is usually in the range from 0.5 to 3, preferably in the range from 0.1 to 2.5 and particularly preferably in the range from 1 to 2. Particular preference is given to methylated, ethylated or propylated α-, β-, γ-cyclodextrins with an average degree of etherification of from 1.5 to 2.2. Also suitable are cyclodextrin esters which are obtainable by reacting cyclodextrins with acid chlorides such as carbonyl or sulfonyl chlorides.

Particularly suitable are carbonyl chlorides such as acetyl chloride, acryloyl chloride, methacryloyl chloride or benzoyl chloride.

Also suitable are polymer-modified cyclodextrins, that is to say cyclodextrins which are incorporated into the main chain of polymers and/or cyclodextrins which have been attached to side chains of polymers or are themselves side chains of polymers. Polymer-modified cyclodextrins in which the cyclodextrin units are arranged in the main chain of the polymer can be obtained, for example, by reacting cyclodextrins with or in the presence of suitable coupling or crosslinking reagents, for example as described in Helv. Chim. Acta, Vol. 48, (1965), p. 1225. Polymer-modified cyclodextrins in which the cyclodextrin units are side chain constituents or act as side chains can be obtained, for example, by cyclodextrins modified with polymerizable groups being polymerized with other comonomers, for example by polymerizing cyclodextrin (meth)acrylates in the presence of other ethylenically unsaturated monomers or by free-radical grafting of cyclodextrin (meth)acrylates onto polymers with free hydroxyl groups such as, for example, polyvinyl alcohol. Another possibility for preparing polymer-modified cyclodextrins with the cyclodextrin units on side groups or as side groups of polymers is to react cyclodextrins, deprotonated cyclodextrins or their alkali metal salts with polymers which have complementary reactive groups such as, for example, anhydride, isocyanate, acid halide or epoxy groups or halogens.

Preferred cyclodextrines are hydroxyalkyl-cyclodextrines, such as hydroxypropyl-β-cyclodextrin.

Suitable lipids may be selected from waxes, tri-, di-, and monoglycerides and phospholipids.

The preferred matrix-forming agents are pharmaceutically acceptable polymers.

The pharmaceutically acceptable polymers may be selected from water-soluble polymers, water-dispersible polymers or water-swellable polymers or any mixture thereof. Polymers are considered water-soluble if they form a clear homogeneous solution in water. When dissolved at 20° C. in an aqueous solution at 2% (w/v), the water-soluble polymer preferably has an apparent viscosity of 1 to 5000 mPa·s, more preferably of 1 to 700 mPa·s, and most preferably of 5 to 100 mPa·s. Water-dispersible polymers are those that, when contacted with water, form colloidal dispersions rather than a clear solution. Upon contact with water or aqueous solutions, water-swellable polymers typically form a rubbery gel. Water-soluble polymers are preferred.

Preferably, the pharmaceutically acceptable polymer employed in the invention has a Tg of at least 40° C., preferably at least +50° C., most preferably from 80° to 180.° C. “Tg” means glass transition temperature. Methods for determining Tg values of the organic polymers are described in “Introduction to Physical Polymer Science”, 2nd Edition by L. H. Sperling, published by John Wiley & Sons, Inc., 1992. The Tg value can be calculated as the weighted sum of the Tg values for homopolymers derived from each of the individual monomers, i, that make up the polymer: Tg=ΣWᵢXᵢ where W is the weight percent of monomer i in the organic polymer, and X is the Tg value for the homopolymer derived from monomer i. Tg values for the homopolymers may be taken from “Polymer Handbook”, 2nd Edition by J. Brandrup and E. H. Immergut, Editors, published by John Wiley & Sons, Inc., 1975.

Various additives contained in the solid dispersion product or even the active ingredient(s) itself may exert a plasticizing effect on the polymer and thus depress the Tg of the polymer such that the final solid dispersion product has a somewhat lower Tg than the starting polymer used for its preparation. In general, the final solid dispersion product has a Tg of 10° C. or higher, preferably 15° C. or higher, more preferably 20° C. or higher and most preferred 30° C. or higher.

For example, preferred pharmaceutically acceptable polymers can be selected from the group comprising

homopolymers and copolymers of N-vinyl lactams, especially homopolymers and co-polymers of N-vinyl pyrrolidone, e.g. polyvinylpyrrolidone (PVP), copolymers of N-vinyl pyrrolidone and vinyl acetate or vinyl propionate,
cellulose esters, cellulose ethers and cellulose ether-esters, in particular methylcellulose and ethylcellulose, hydroxyalkylcelluloses, in particular hydroxypropylcellulose, hydroxyalkylalkylcelluloses, in particular hydroxypropylmethylcellulose, cellulose phthalates or succinates, in particular cellulose acetate phthalate and hydroxypropylmethyl-cellulose phthalate, hydroxypropylmethylcellulose succinate or hydroxypropylmethylcellulose acetate succinate;
high molecular polyalkylene oxides such as polyethylene oxide and polypropylene oxide and copolymers of ethylene oxide and propylene oxide,
polyvinyl alcohol-polyethylene glycol-graft copolymers (available as Kollicoat® IR from BASF SE, Ludwigshafen, Germany);
polyacrylates and polymethacrylates such as methacrylic acid/ethyl acrylate copolymers, methacrylic acid/methyl methacrylate copolymers, butyl methacrylate/2-dimethyl-aminoethyl methacrylate copolymers, poly(hydroxyalkyl acrylates), poly(hydroxyalkyl methacrylates),
polyacrylamides,
vinyl acetate polymers such as copolymers of vinyl acetate and crotonic acid, partially hydrolyzed polyvinyl acetate (also referred to as partially saponified “polyvinyl alcohol”),
polyvinyl alcohol,
oligo- and polysaccharides such as carrageenans, galactomannans and xanthan gum, or mixtures of one or more thereof.

Among these, homopolymers or copolymers of N-vinyl pyrrolidone, in particular a copolymer of N-vinyl pyrrolidone and vinyl acetate, are preferred. A particularly preferred polymer is a copolymer of 60% by weight of the copolymer, N-vinyl pyrrolidone and 40% by weight of the copolymer, vinyl acetate. Different grades of commercially available N-vinyl pyrrolidone homopolymers (also referred to as polyvinylpyrrolidone or PVP) are PVP K-12, PVP K-15, PVP K-17, PVP K-20, PVP K-30, PVP K-60, PVP K-90 and PVP K-120. The K-value referred to in this nomenclature is calculated by Fikentscher's formula from the viscosity of the PVP in aqueous solution, relative to that of water. All of these may suitably be used, with PVP K-12, PVP K-15, PVP K-17, PVP K-20, and PVP K-30 being especially preferred.

A further polymer which can be suitably used is Kollidon® SR (available from BASF SE, Ludwigshafen, Germany) which comprises a mixture of PVP and polyvinylacetate.

The term “pharmaceutically acceptable surfactant” as used herein refers to a pharmaceutically acceptable non-ionic surfactant. The surfactant may effectuate an instantaneous emulsification of the active agent released from the dosage form and/or prevent precipitation of the active ingredient in the aqueous fluids of the gastrointestinal tract. A single surfactant as well as combinations of surfactants may be used. According to an embodiment of the invention, the solid dispersion product comprises a combination of two or more pharmaceutically acceptable surfactants.

Preferred surfactants are selected from sorbitan fatty acid esters, polyalkoxylated fatty acid esters such as, for example, polyalkoxylated glycerides, polyalkoxylated sorbitan fatty acid esters or fatty acid esters of polyalkylene glycols, polyalkoxylated ethers of fatty alcohols, tocopheryl compounds or mixtures of two or more thereof. A fatty acid chain in these compounds ordinarily comprises from 8 to 22 carbon atoms. The polyalkylene oxide blocks comprise on average from 4 to 50 alkylene oxide units, preferably ethylene oxide units, per molecule.

Suitable sorbitan fatty acid esters are sorbitan monolaurate, sorbitan monopalmitate, sorbitan monostearate (Span® 60), sorbitan monooleate (Span® 80), sorbitan tristearate, sorbitan trioleate, sorbitan monostearate, sorbitan monolaurate or sorbitan monooleate.

Examples of suitable polyalkoxylated sorbitan fatty acid esters are polyoxyethylene (20) sorbitan monolaurate, polyoxyethylene (20) sorbitan monopalmitate, polyoxyethylene (20) sorbitan monostearate, polyoxyethylene (20) sorbitan monooleate (Tween® 80), polyoxyethylene (20) sorbitan tristearate (Tween® 65), polyoxyethylene (20) sorbitan trioleate (Tween® 85), polyoxyethylene (4) sorbitan monostearate, polyoxyethylene (4) sorbitan monolaurate or polyoxyethylene (4) sorbitan monooleate.

Suitable polyalkoxylated glycerides are obtained for example by alkoxylation of natural or hydrogenated glycerides or by transesterification of natural or hydrogenated glycerides with polyalkylene glycols. Commercially available examples are polyoxyethylene glycerol ricinoleate 35, polyoxyethylene glycerol trihydroxystearate 40 (Cremophor® RH40, BASF SE) and polyalkoxylated glycerides like those obtainable under the proprietary names Gelucire® and Labrafil® from Gattefosse, e.g. Gelucire® 44/14 (lauroyl macrogol 32 glycerides prepared by transesterification of hydrogenated palm kernel oil with PEG 1500), Gelucire® 50/13 (stearoyl macrogol 32 glycerides, prepared by transesterification of hydrogenated palm oil with PEG 1500) or Labrafil M1944 CS (oleoyl macrogol 6 glycerides prepared by transesterification of apricot kernel oil with PEG 300).

A suitable fatty acid ester of polyalkylene glycols is, for example, PEG 660 hydroxy-stearic acid (polyglycol ester of 12-hydroxystearic acid (70 mol %) with 30 mol % ethylene glycol).

Suitable polyalkoxylated ethers of fatty alcohols are, for example, PEG (2) stearyl ether (Brij® 72), macrogol 6 cetylstearyl ether or macrogol 25 cetylstearyl ether.

In general, the tocopheryl compound corresponds to the formula below

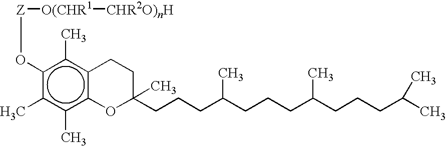

wherein Z is a linking group, R¹ and R² are, independently of one another, hydrogen or C₁-C₄ alkyl and n is an integer from 5 to 100, preferably 10 to 50. Typically, Z is the residue of an aliphatic dibasic acid such as glutaric, succinic, or adipic acid. Preferably, both R¹ and R² are hydrogen.

The preferred tocopheryl compound is alpha tocopheryl polyethylene glycol succinate, which is commonly abbreviated as vitamin E TPGS. Vitamin E TPGS is a water-soluble form of natural-source vitamin E prepared by esterifying d-alpha-tocopheryl acid succinate with polyethylene glycol 1000. Vitamin E TPGS is available from Eastman Chemical Company, Kingsport, Tenn., USA and is listed in the US pharmacopoeia (NF).

It was found that surfactants or combination of surfactants having a defined HLB (hydrophilic lipophilic balance) value are preferred over other solubilizers.

The HLB system (Fiedler, H. B., Encyclopedia of Excipients, 5^{th} ed., Aulendorf: ECV-Editio-Cantor-Verlag (2002)) attributes numeric values to surfactants, with lipophilic substances receiving lower HLB values and hydrophilic substances receiving higher HLB values.

In preferred embodiments, the pharmaceutically acceptable surfactant comprises at least one surfactant having an HLB value of 10 or more.

Solubilizers having an HLB value of 10 or more may be selected from Gelucire® 44/14 (HLB 14), Cremophor® RH40 (HLB 13), Tween® 65 (HLB 10.5), Tween® 85 (HLB 11). Preferred high HLB solubilizers are tocopheryl compounds having a polyalkylene glycol moiety.

In a preferred embodiment, a combination of solubilizers is used which comprises (i) at least one tocopheryl compound having a polyalkylene glycol moiety, preferably alpha tocopheryl polyethylene glycol succinate, and (ii) at least one polyalkoxylated polyol fatty acid ester. The tocopheryl compound preferably is alpha tocopheryl polyethylene glycol succinate. The polyalkoxylated polyol fatty acid ester preferably is a polyalkoxylated glyceride. The mass ratio of tocopheryl compound and polyalkoxylated polyol fatty acid ester preferably is in the range of from 0.2:1 to 1:1.

In an embodiment, the active agent is an N-aryl urea-based active agent. N-aryl urea-based active agents are biologically active compounds which comprise at least one urea moiety in their molecular structure wherein one or both nitrogen atoms are substituted by an aryl group, and which exert a local physiological effect, as well as those which exert a systemic effect, after oral administration. The aryl group may be a carbocyclic or heterocyclic aromatic group or a fused carbocyclic or heterocyclic aromatic group. Attachment to the nitrogen atom is usually via a carbon atom of the aryl group. A fused aromatic group may be linked to the nitrogen atom via an aromatic or non-aromatic carbon atom. The aryl group may, of course, be substituted by further substituents.

Generally, the N-aryl urea-based active agent is represented by the general formula

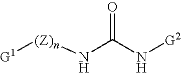

wherein
G¹ and G² are, independently from one another, a carbocyclic ring selected from phenyl, naphthyl, benzocyclobutanyl, dihydronaphthyl, tetrahydronaphthyl, benzocycloheptanyl, benzocycloheptenyl, indanyl and indenyl;
   a ring system selected from (dihydro)benzoxazinyl, benzimidazolyl, indazolyl, benzothiazolyl, benzooxazolyl, benzisoxazolyl, benzofuranyl, (dihydro)benzopyranyl, benzodioxolyl, (dihydro)quinaldinyl, (dihydro)quinazolinyl, (dihydro)quinoxalinyl, (dihydro)isoquinolinyl, (dihydro)quinolinyl, indolyl, isoindolyl, indolinyl, purinyl, tetrahydroquinolinyl, indazolyl, imidazo-pyridinyl, pyrazolopyridinyl, pyrazolo-pyrimidinyl, pyrrolo-pyrimidinyl, pyrrolo-pyridinyl, pyridopyrazinyl, pyrido-pyrimidinyl, pyrido-oxazinyl, pyrido-thiazinyl, pyrido-oxazolyl, pyrido-thioxazolyl, pyrimido-pyrimidine, pteridinyl, cinnolinyl and naphthyridinyl;
wherein G¹ or G² or both may be substituted by one or more substituents, e.g., selected from the group consisting of C₁₋₆ branched or unbranched alkyl, C₁₋₆ haloalkyl, C₁₋₆ branched or unbranched acyl, C₁₋₆ branched or unbranched alkoxy, halogen, C₁₋₆ branched or unbranched alkyloxycarbonyl, hydroxy, amino, mono- or di-(C₁₋₄ alkyl)amino, mono- or di-(C₁₋₄ alkyl)amino-SO₂, cyano, nitro or H₂NSO₂,
Z is 1,4-phenylene, and
n is 0 or 1,
or the pharmaceutically acceptable salts, esters, isomers, hydrates or solvates thereof

In this nomenclature, the prefix “(dihydro)” is intended to mean either the dihydro compound or the aromatic compound without the prefix; thus (dihydro)benzoxazinyl means either dihydrobenzoxazinyl or benzoxazinyl, etc.

In an embodiment, the active agent is at least one compound of formula (I)

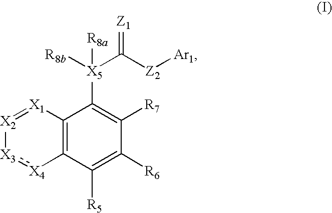

or a pharmaceutically acceptable salt or prodrug thereof, wherein
--- is absent or a single bond;
X₁ is N or CR₁;
X₂ is N or CR₂;
X₃ is N, NR₃, or CR₃;
X₄ is a bond, N, or CR₄;
X₅ is N or C;
provided that at least one of X₁, X₂, X₃, and X₄ is N;
Z₁ is O, NH, or S;
Z₂ is a bond, NH, or O;
Ar₁ is selected from the group consisting of

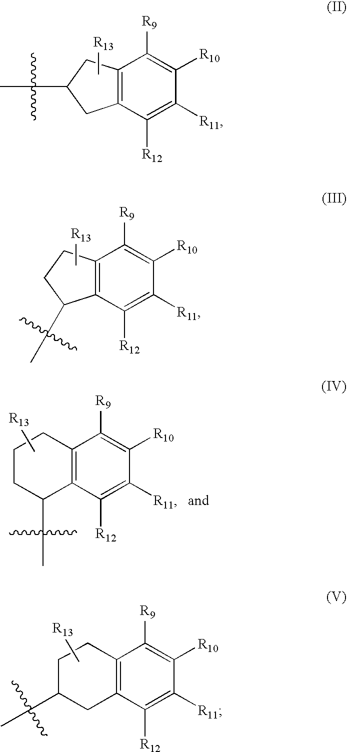

R₁, R₃, R₅, R₆, and R₇ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl;
R₂ and R₄ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)alkylcarbonyl, (Z_{A}Z_{B}N)carbonyl, (Z_{A}Z_{B}N)carbonylalkyl, (Z_{A}Z_{B}N)sulfonyl, (Z_{A}Z_{B}N)C(═NH)—, (Z_{A}Z_{B}N)C(═NCN)NH— and (Z_{A}Z_{B}N)C(═NH)NH—;
R₈ₐ is hydrogen or alkyl;
R_{8b} is absent, hydrogen, alkoxy, alkoxycarbonylalkyl, alkyl, alkylcarbonyloxy, alkylsulfonyloxy, halogen, or hydroxy;
R₉, R₁₀, R₁₁, and R₁₂ are each individually selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, aryl, carboxy, carboxyalkyl, cyano, cyanoalkyl, formyl, formylalkyl, haloalkoxy, haloalkyl, haloalkylthio, halogen, heteroaryl, heterocycle, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl, wherein Z_{A} and Z_{B} are each independently hydrogen, alkyl, alkylcarbonyl, formyl, aryl, or arylalkyl, provided that at least one of R₉, R₁₀, R₁₁, or R₁₂ is other than hydrogen, or R₁₀ and R₁₁ taken together with the atoms to which they are attached form a cycloalkyl, cycloalkenyl, or heterocycle ring;
R₁₃ is selected from the group consisting of hydrogen, alkyl, aryl, heteroaryl, and halogen;
R_{A} is hydrogen or alkyl; and
R_{B} is alkyl, aryl, or arylalkyl;
provided that R_{8b} is absent when X₅ is N.

In an embodiment of the present invention, the active agent is at least one compound of formula (I) wherein --- is absent; X₁ is CR₁,; X₂ is N; X₃ is NR₃; X₄ is a bond; X₅ is N; Z₁ is O; Z₂ is NH; Ar₁ is selected from the group consisting of

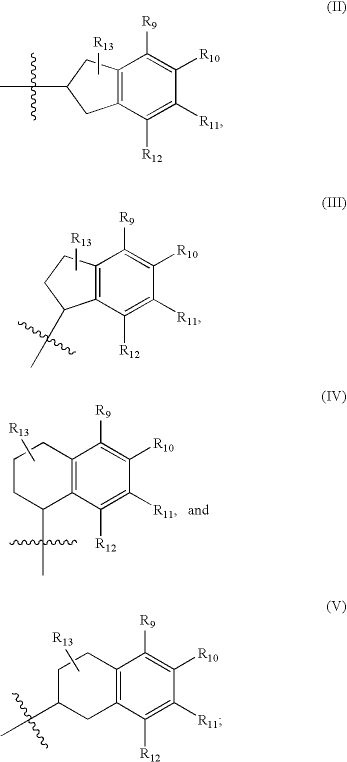

R_{8b} is absent; and R₁, R₃, R₅, R₆, R₇, R₈ₐ, R₉, R₁₀, R₁₁, R₁₂ and R₁₃ are as defined in formula (I).

In another embodiment of the present invention, the active agent is at least one compound of formula (I) wherein --- is absent; X₁ is CR₁; X₂ is N; X₃ is NR₃; X₄ is a bond; X₅ is N; Z₁ is O; Z₂ is NH; Ar₁ is selected from the group consisting of

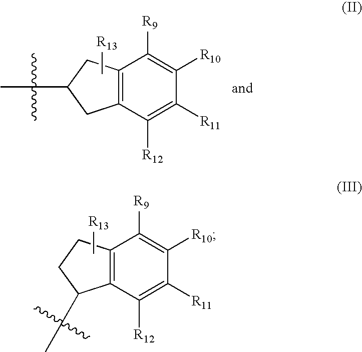

R₁ is selected from the group consisting of hydrogen, alkyl, halogen, and hydroxyalkyl; R₃, R₅, R₆, R₇, and R₈ₐ are hydrogen; R_{8b} is absent; and R₉, R₁₀, R₁₁, R₁₂ and R₁₃ are as defined in formula (I).

In another embodiment of the present invention, the active agent is at least one compound of formula (I) wherein --- is absent; X₁ is CR₁; X₂ is N; X₃ is NR₃; X₄ is a bond; X₅ is N; Z₁ is O; Z₂ is NH; Ar₁is selected from the group consisting of

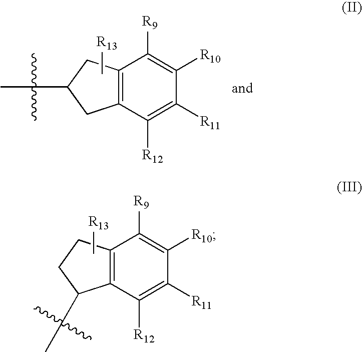

R₁ is selected from the group consisting of hydrogen, alkyl and hydroxyalkyl; R₃, R₅, R₆, R₇, and R₈ₐ are hydrogen; at least one of R₉, R₁₀, R₁₁, and R₁₂ are independently selected from the group consisting of alkyl, alkoxy, alkoxyalkyl, aryl, cyanoalkyl, halogen, haloalkyl, haloalkoxy and heterocycle; R_{8b} is absent; and R₁₃ is as defined in formula (I).

In another embodiment of the present invention, the active agent is at least one compound of formula (I) wherein --- is absent; X₁ is CR₁; X₂ is N; X₃ is NR₃; X₄ is a bond; X₅ is N; Z₁ is O; Z₂ is NH; Ar₁is selected from the group consisting of

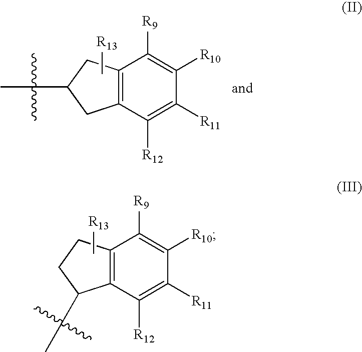

R₁ is selected from the group consisting of hydrogen, alkyl and hydroxyalkyl; R₃, R₅, R₆, R₇, and R₈ₐ are hydrogen; at least one of R₉, R₁₀, R₁₁, and R₁₂ are independently selected from the group consisting of alkyl, alkoxy, alkoxyalkyl, cyanoalkyl, halogen, haloalkyl, and haloalkoxy; R_{8b} is absent; and R₁₃ is as defined in formula (I).

In another embodiment, the active agent is at least one compound of formula (I), wherein Ar₁is

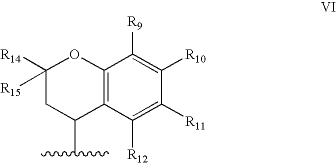

R₁₄ and R₁₅ are each individually selected from the group consisting of hydrogen and alkyl, or R₁₄ and R₁₅ taken together with the atom to which they are attached form a cycloalkyl ring,
   and X₁, X₂, X₃, X₄, X₅, Z₁, Z₂, R₁, R₂, R₃, R₄, R₅, R₆, R₇, R₈ₐ, R_{8b}, R₉, R₁₀, Ran and R₁₂ are as defined in formula (I).

In another embodiment, the active agent is at least one compound of formula (VII),

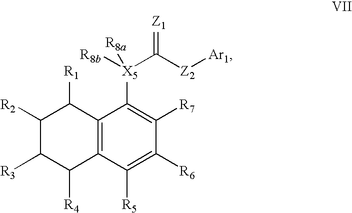

wherein Ar₁is

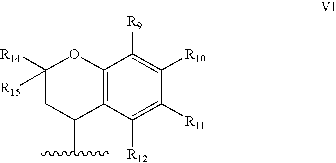

R₁₄ and R₁₅ are each individually selected from the group consisting of hydrogen and alkyl, or R₁₄ and R₁₅ taken together with the atom to which they are attached form a cycloalkyl ring,
   and X₅, Z₁, Z₂, R₁, R₂, R₃, R₄, R₅, R₆, R₇, R₈ₐ, R_{8b}, R₉, R₁₀, R₁₁ and R₁₂ are as defined in formula (I).
   Compounds contemplated within the genus include:
N-(5-tert-butyl-2,3-dihydro-1H-inden-1-yl)-N′-5-isoquinolinylurea;
N-(5-tert-butyl-2,3-dihydro-1H-inden-1-yl)-N′-(3-methyl-5-isoquinolinyl)urea;
(+) N-(5-tert-butyl-2,3-dihydro-1H-inden-1-yl)-N′-(3-methyl-5-isoquinolinyl)urea;
(−) N-(5-tert-butyl-2,3-dihydro-1H-inden-1-yl)-N′-(3-methyl-5-isoquinolinyl)urea;
(−) N-(5-tert-butyl-2,3-dihydro-1H-inden-1-yl)-N′-5-isoquinolinylurea;
(+) N-(5-tert-butyl-2,3-dihydro-1H-inden-1-yl)-N′-5-isoquinolinylurea;
N-(5-bromo-2,3-dihydro-1H-inden-1-yl)-N′-5-isoquinolinylurea;
methyl 4-({[(5-tert-butyl-2,3-dihydro-1H-inden-1-yl)amino]carbonyl}amino)-1H-indazole-1-carboxylate;
N-(5-tert-butyl-2,3-dihydro-1H-inden-1-yl)-N′-1H-indazol-4-ylurea (ABT-102);
methyl 4-[({[(1S)-5-tert-butyl-2,3-dihydro-1H-inden-1-yl]amino}carbonyl)amino]-1H-indazole-1-carboxylate;
methyl 4-[({[(1R)-5-tert-butyl-2,3-dihydro-1H-inden-1-yl]amino}carbonyl)amino]-1H-indazole-1-carboxylate;
N-[(1S)-5-tert-butyl-2,3-dihydro-1H-inden-1-yl]-N′-1H-indazol-4-ylurea;
N-[(1R)-5-tert-butyl-2,3-dihydro-1H-inden-1-yl]-N′-1H-indazol-4-ylurea;
methyl 4-[({[5-(trifluoromethyl)-2,3-dihydro-1H-inden-1-yl]amino}carbonyl)amino]-1H-indazole-1-carboxylate;
N-1H-indazol-4-yl-N′-[5-(trifluoromethyl)-2,3-dihydro-1H-inden-1-yl]urea;
methyl 4-({[(5-piperidin-1-yl-2,3-dihydro-1H-inden-1-yl)amino]carbonyl}amino)-1H-indazole-1-carboxylate;
N-1H-indazol-4-yl-N′-(5-piperidin-1-yl-2,3-dihydro-1H-inden-1-yl)urea;
methyl 4-({[(5-hexahydro-1H-azepin-1-yl-2,3-dihydro-1H-inden-1-yl)amino]carbonyl}amino)-1H-indazole-1-carboxylate;
N-(5-hexahydro-1H-azepin-1-yl-2,3-dihydro-1H-inden-1-yl)-N′-1H-indazol-4-ylurea;
N-1H-indazol-4-yl-N′-[(1R)-5-piperidin-1-yl-2,3-dihydro-1H-inden-1-yl]urea;
N-1H-indazol-4-yl-N′-[(1S)-5-piperidin-1-yl-2,3-dihydro-1H-inden-1-yl]urea;
isopropyl 4-({[(5-tert-butyl-2,3-dihydro-1H-inden-1-yl)amino]carbonyl}amino)-1H-indazole-1-carboxylate; and
isobutyl 4-({[(5-tert-butyl-2,3-dihydro-1H-inden-1-yl)amino]carbonyl}amino)-1H-indazole-1-carboxylate;
   All these compounds have been previously prepared and described in U.S. Pat. No. 7,015,233.

Dosage forms wherein the active agent is a compound of formula (I) or (VII) or a pharmaceutically acceptable salt or prodrug thereof may be used for treating a disorder by inhibiting vanilloid receptor subtype. The disorder may be selected from pain, bladder overactivity, urinary incontinence and inflammatory thermal hyperalgesia.

As used throughout this specification and the appended claims, the following terms have the following meanings:
The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.
The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.
The term “alkoxyalkoxy” as used herein, means an alkoxy group, as defined herein, appended to the parent molecular moiety through an alkoxy group, as defined herein. Representative examples of alkoxyalkoxy include, but are not limited to, methoxymethoxy, ethoxymethoxy and 2-ethoxyethoxy.
The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.
The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.
The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.
The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.
The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.
The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.
The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.
The term “alkylsulfonyl” as used herein, means an alkyl group, as defined herein, appended to the parent molecular moiety through a sulfonyl group. Representative examples of alkylsulfonyl include, but are not limited to, methylsulfonyl and ethylsulfonyl.
The term “alkylthio” as used herein, means an alkyl group, as defined herein, appended to the parent molecular moiety through a sulfur atom. Representative examples of alkylthio include, but are not limited, methylsulfanyl, ethylsulfanyl, tert-butylsulfanyl, and hexylsulfanyl.
The term “alkynyl” as used herein, means a straight or branched chain hydro-carbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.
The term “aryl” as used herein, means a phenyl group, or a bicyclic or a tricyclic fused ring system wherein one or more of the fused rings is a phenyl group. Bicyclic fused ring systems are exemplified by a phenyl group fused to a cycloalkyl group, as defined herein, or another phenyl group. Tricyclic fused ring systems are exemplified by a bicyclic fused ring system fused to a cycloalkyl group, as defined herein, or another phenyl group. Representative examples of aryl include, but are not limited to, anthracenyl, azulenyl, fluorenyl, indenyl, naphthyl, phenyl and tetrahydronaphthyl.
The term “cycloalkyl” as used herein, means a saturated monocyclic ring system containing from 3 to 8 carbon atoms. Examples of cycloalkyl include cyclopropyl, cyclobutyl, cyclopentyl, cyclohexyl, cycloheptyl, and cyclooctyl.
The term “formyl” as used herein, means a —C(O)H group.
The term “halo” or “halogen” as used herein, means —Cl, —Br, —I or —F.
The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, 2-chloro-3-fluoropentyloxy, and pentafluoroethoxy.
The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.
The term “heterocycle,” as used herein, refers to a three, four, five, six, seven, or eight membered ring containing one or two heteroatoms independently selected from the group consisting of nitrogen, oxygen, and sulfur. The three membered ring has zero double bonds. The four and five membered ring has zero or one double bond. The six membered ring has zero, one, or two double bonds. The seven and eight membered rings have zero, one, two, or three double bonds. The heterocycle groups of the present invention can be attached to the parent molecular moiety through a carbon atom or a nitrogen atom. Representative examples of heterocycle include, but are not limited to, azabicyclo[2.2.1]heptanyl, azabicyclo[2.2.1.]octanyl, azetidinyl, hexahydro-1H-azepinyl, hexahydroazocin-(2H)-yl, indazolyl, morpholinyl, octahydroisoquinoline, piperazinyl, piperidinyl, pyridinyl, pyrrolidinyl, and thiomorpholinyl.
The term “mercaptoalkyl” as used herein, means a mercapto group appended to the parent molecular moiety through an alkyl group, as defined herein. Representative examples of mercaptoalkyl include, but are not limited to, 2-mercaptoethyl and 3-mercaptopropyl.

In an embodiment of the invention, the active agent is 1-((R)-5-tert-butyl-indan-1-yl)-3-(1H-indazol-4-yl)-urea (ABT102)

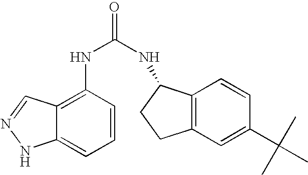

or salts or hydrates or solvates thereof.

In another embodiment of the invention, the active agent is selected from one or more of the following compounds:

N-[(4R)-6-fluoro-2,2-dimethyl-3,4-dihydro-2H-chromen-4-yl]-N′-(1-methyl-1H-indazol-4-yl)urea;
N-[(4R)-6-fluoro-2,2-dimethyl-3,4-dihydro-2H-chromen-4-yl]-N′-1H-indazol-4-ylurea;
N-[(4R)-6-fluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-6-fluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-6-fluoro-2,2-dimethyl-3,4-dihydro-2H-chromen-4-yl]-N′-isoquinolin-5-ylurea;
N-[(4R)-6-fluoro-3,3′,4,4′-tetrahydro-2′ H-spiro[chromene-2,1′-cyclobutan]-4-yl]-N′-[(7R)-7-hydroxy-5,6,7,8-tetrahydronaphthalen-1-yl]urea;
N-[(4R)-6-fluoro-3,3′,4,4′-tetrahydro-2′ H-spiro[chromene-2,1′-cyclobutan]-4-yl]-N′-(1-methyl-1H-indazol-4-yl)urea;
N-[(4R)-6-fluoro-3,3′,4,4′-tetrahydro-2′ H-spiro[chromene-2,1′-cyclobutan]-4-yl]-N′-1H-indazol-4-ylurea;
N-[(4R)-6-fluoro-3,3′,4,4′-tetrahydro-2′ H-spiro[chromene-2,1′-cyclobutan]-4-yl]-N′-[(7S)-57-hydroxy-5,6,7,8-tetrahydronaphthalen-1-yl]urea;
N-[(4S)-6-fluoro-3,3′,4,4′-tetrahydro-2′H-spiro[chromene-2,1′-cyclobutan]-4-yl]-N′-[(7S)-7-hydroxy-5,6,7,8-tetrahydronaphthalen-1-yl]urea;
10 N-[(4S)-6-fluoro-3,3′,4,4′-tetrahydro-2′H-spiro[chromene-2,1′-cyclobutan]-4-yl]-N′-[(7R)-7-hydroxy-5,6,7,8-tetrahydronaphthalen-1-yl]urea;
N-[(4R)-6-fluoro-3,4-dihydro-2H-chromen-4-yl]-N′-(1-methyl-1H-indazol-4-yl)urea;
N-[(4R)-6-fluoro-3,4-dihydro-2H-chromen-4-yl]-N′-isoquinolin-5-ylurea;
N-[(4R)-6-fluoro-3,4-dihydro-2H-chromen-4-yl]-N′-[(7R)-7-hydroxy-5,6,7,8-tetrahydronaphthalen-1-yl]urea;
N-[(4R)-6-fluoro-3,4-dihydro-2H-chromen-4-yl]-N′-[(7S)-7-hydroxy-5,6,7,8-tetrahydronaphthalen-1-yl]urea;
N-[(4R)-6,8-difluoro-2,2-dimethyl-3,4-dihydro-2H-chromen-4-yl]-N′-(1-methyl-1H-indazol-4-yl)urea;
N-[(4R)-6,8-difluoro-2,2-dimethyl-3,4-dihydro-2H-chromen-4-yl]-N′-isoquinolin-5-ylurea;
N-[(4R)-6,8-difluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-6,8-difluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-8-fluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-8-fluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-8-fluoro-2,2-dimethyl-3,4-dihydro-2H-chromen-4-yl]-N′-isoquinolin-5-ylurea;
N-[(4R)-7-fluoro-2,2-dimethyl-3,4-dihydro-2H-chromen-4-yl]-N′-isoquinolin-5-ylurea;
N-[(4R)-7-fluoro-2,2-dimethyl-3,4-dihydro-2H-chromen-4-yl]-N′-(1-methyl-1H-indazol-4-yl)urea;
N-[(4R)-8-fluoro-2,2-dimethyl-3,4-dihydro-2H-chromen-4-yl]-N′-(1-methyl-1H-indazol-4-yl)urea;
N-[(4R)-2,2-diethyl-6-fluoro-3,4-dihydro-2H-chromen-4-yl]-N′-(1-methyl-1H-indazol-4-yl)urea;
N-[(4R)-2,2-dimethyl-3,4-dihydro-2H-chromen-4-yl]-N′-isoquinolin-5-ylurea;
N-[(4R)-2,2-diethyl-6-fluoro-3,4-dihydro-2H-chromen-4-yl]-N′-isoquinolin-5-ylurea;
N-[(4R)-7-fluoro-2,2-dimethyl-3,4-dihydro-2H-chromen-4-yl]-N′-1H-indazol-4-ylurea;
N-[(4R)-7-fluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-7-fluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-8-fluoro-2,2-dimethyl-3,4-dihydro-2H-chromen-4-yl]-N′-1H-indazol-4-ylurea;
N-[(4R)-2,2-dimethyl-7-(trifluoromethyl)-3,4-dihydro-2H-chromen-4-yl]-N′-(-methyl-1H-indazol-4-yl)urea;
N-[(4R)-2,2-dimethyl-7-(trifluoromethyl)-3,4-dihydro-2H-chromen-4-yl]-N′-isoquinolin-5-ylurea;
N-[(4R)-6-fluoro-2,2-dimethyl-3,4-dihydro-2H-chromen-4-yl]-N′-(3-methylisoquinolin-5-yl)urea;
N-[(4R)-2,2-dimethyl-3,4-dihydro-2H-chromen-4-yl]-N′-[(7R)-7-hydroxy-5,6,7,8-tetrahydronaphthalen-1-yl]urea;
N-[(4R)-2,2-dimethyl-3,4-dihydro-2H-chromen-4-yl]-N′-[(7S)-7-hydroxy-5,6,7,8-tetrahydronaphthalen-1-yl]urea;
N-[(4R)-6-fluoro-2,2-dimethyl-3,4-dihydro-2H-chromen-4-yl]-N′-isoquinolin-8-ylurea;
N-[(4R)-2,2-dimethyl-7-(trifluoromethoxy)-3,4-dihydro-2H-chromen-4-yl]-N′-(1-methyl-1H-indazol-4-yl)urea;
N-[(4R)-2,2-dimethyl-7-(trifluoromethoxy)-3,4-dihydro-2H-chromen-4-yl]-N′-isoquinolin-5-ylurea;
N-[(4R)-2,2-dimethyl-7-(trifluoromethyl)-3,4-dihydro-2H-chromen-4-yl]-N′- H-indazol-4-ylurea;
N-[(4R)-2,2-dimethyl-8-(trifluoromethyl)-3,4-dihydro-2H-chromen-4-yl]-N′-(-methyl-1H-indazol-4-yl)urea;
N-[(4R)-2,2-dimethyl-7-(trifluoromethyl)-3,4-dihydro-2H-chromen-4-yl]-N′-isoquinolin-8-ylurea;
N-[(4R)-2,2-dimethyl-8-(trifluoromethyl)-3,4-dihydro-2H-chromen-4-yl]-N′-isoquinolin-5-ylurea;
N-[(4R)-2,2-dimethyl-8-(trifluoromethyl)-3,4-dihydro-2H-chromen-4-yl]-N′- H-indazol-4-ylurea;
N-[(4R)-2,2-diethyl-7-fluoro-3,4-dihydro-2H-chromen-4-yl]-N′-(1-methyl-1H-indazol-4-yl)urea;
N-[(4R)-2,2-dimethyl-8-(trifluoromethyl)-3,4-dihydro-2H-chromen-4-yl]-N′-[(7R)-7-hydroxy-5,6,7,8-tetrahydronaphthalen-1-yl]urea;
N-[(4R)-2,2-diethyl-7-fluoro-3,4-dihydro-2H-chromen-4-yl]-N′-isoquinolin-5-ylurea;
N-[(4R)-2,2-diethyl-7-(trifluoromethyl)-3,4-dihydro-2H-chromen-4-yl]-N′-(-methyl-1H-indazol-4-yl)urea;

N-[(4R)-2,2-diethyl-8-fluoro-3,4-dihydro-2H-chromen-4-yl]-N′-(1-methyl-1H-indazol-4-yl)urea;
N-[(4R)-2,2-diethyl-7-(trifluoromethyl)-3,4-dihydro-2H-chromen-4-yl]-N′-isoquinolin-5-ylurea;
N-[(4R)-2,2-diethyl-8-fluoro-3,4-dihydro-2H-chromen-4-yl]-N′-[(7R)-7-hydroxy-5,6,7,8-tetrahydronaphthalen-1-yl]urea;
N-[(4R)-2,2-dimethyl-7-(trifluoromethyl)-3,4-dihydro-2H-chromen-4-yl]-N′-[(7R)-7-hydroxy-5,6,7,8-tetrahydronaphthalen-1-yl]urea;
N-[(4R)-2,2-diethyl-6-fluoro-3,4-dihydro-2H-chromen-4-yl]-N′-[(7R)-7-hydroxy-5,6,7,8-tetrahydronaphthalen-1-yl]urea;
N-[(4R)-2,2-diethyl-8-(trifluoromethoxy)-3,4-dihydro-2H-chromen-4-yl]-N′-(-methyl-1H-indazol-4-yl)urea;
N-[(4R)-2,2-diethyl-6-fluoro-3,4-dihydro-2H-chromen-4-yl]-N′-(3-methylisoquinolin-5-yl)urea;
N-[(4R)-2,2-diethyl-8-fluoro-3,4-dihydro-2H-chromen-4-yl]-N′-isoquinolin-5-ylurea;
N-[(4R)-2,2-diethyl-6-fluoro-3,4-dihydro-2H-chromen-4-yl]-N′-1H-indazol-4-ylurea;
N-(-methyl-1H-indazol-4-yl)-N′-[(4R)-8-(trifluoromethyl)-3,4-dihydro-2H-chromen-4-35 yl]urea;
N-[(4R)-2,2-diethyl-6,8-difluoro-3,4-dihydro-2H-chromen-4-yl]-N′-(1-methyl-1H-indazol-4-yl)urea;
N-[(4R)-6-fluoro-2,2-dipropyl-3,4-dihydro-2H-chromen-4-yl]-N′-(1-methyl-1H-indazol-4-yl)urea;
N-[(4R)-2,2-diethyl-8-fluoro-3,4-dihydro-2H-chromen-4-yl]-N′-(3-methylisoquinolin-5-yl)urea;
N-1H-indazol-4-yl-N′-[(4R)-8-(trifluoromethyl)-3,4-dihydro-2H-chromen-4-yl]urea;
N-isoquinolin-5-yl-N′-[(4R)-8-(trifluoromethyl)-3,4-dihydro-2H-chromen-4-yl]urea.

The solid dispersion product is prepared by a process which comprises
a) preparing a liquid mixture containing the at least one active agent, at least one pharmaceutically acceptable matrix-forming agent, at least one pharmaceutically acceptable surfactant and at least one solvent, and
b) removing the solvent(s) from the liquid mixture to obtain the solid dispersion product.

As described above, at least one filler may advantageously be added to the liquid mixture before removing the solvent(s).

Suitable solvents are those which are capable of dissolving or solubilising the matrix-forming agent. Typically, non-aqueous solvents are used. Any such solvent may be used, however, pharmaceutically acceptable solvents are preferred because traces of solvent may remain in the dried solid dispersion product. Suitably, the solvent may be selected from the group consisting of alkanols, such as methanol, ethanol, isopropanol, n-propanol, isobutanol, n-butanol; hydrocarbons, such as pentane, hexane, cyclohexane, methylcyclohexane, toluene, xylene; halogenated hydrocarbons, such as dichloromethane, trichloromethane, dichloroetane, chlorobenzene; ketons, such as acetone; esters, such as ethyl acetate; ethers, such as dioxane, tetrahydrofurane; and combinations of two or more thereof. Ethanol is particularly preferred due to its availability, dissolving power and pharmaceutical safeness.

The liquid mixture may be prepared by any suitable method of contacting the essential ingredients thereof, i.e. the pharmaceutically acceptable matrix-forming agent, active agent, the pharmaceutically acceptable surfactant and the solvent or combination of solvents. In an embodiment, the liquid mixture is prepared by dissolving the pharmaceutically acceptable matrix-forming agent to obtain a matrix-forming agent solution, and adding the active agent and the pharmaceutically acceptable surfactant to the solution. The dissolved matrix-forming agent may exert a solubility-enhancing effect on the active agent; thus, the solubility of the active agent in the matrix-forming agent solution may be several times higher than its solubility in the solvent alone. Preferably, the active agent is essentially completely dissolved in the liquid mixture.

The liquid mixture has a dry matter content of up to 90% by weight, for example 0.5 to 90% by weight, in most instances 2 to 60% by weight, relative to the total weight of the liquid mixture.

The solvent(s) may be removed by any suitable method known in the art, such as spray-drying, drum drying, belt drying, tray drying, fluid-bed drying or combinations of two or more thereof. For example, the primary solid dispersion powder obtained by spray-drying may be further dried by tray drying (optionally under vacuum) or fluid-bed drying (optionally under vacuum). In an embodiment, removal of the solvent comprises a spray-drying step, optionally in combination with one or more drying steps other than spray-drying.

The residual solvent content in the final solid dispersion product is preferably 5% by weight or less, more preferably 1% by weight or less.

In spray-drying, the liquid to be dried is suspended in a gas flow, e.g., air, i.e. the liquid is converted into a fog-like mist (atomized), providing a large surface area. The atomized liquid is exposed to a flow of hot gas in a drying chamber. The moisture evaporates quickly and the solids are recovered as a powder consisting of fine, hollow spherical particles. Gas inlet temperatures of up to 250° C. or even higher may be used, due to the evaporation the gas temperature drops very rapidly to a temperature of about 30 to 150° C. (outlet temperature of the gas).

The principle of the drum drying process (roller drying) is that a thin film of material is applied to the smooth surface of a continuously rotating, heated metal drum. The film of dried material is continuously scraped off by a stationary knife located opposite the point of application of the liquid material. The dryer consists of a single drum or a pair of drums with or without “satellite” rollers. The drum(s) may be located in a vacuum chamber. Conveniently, the solvent vapours are collected and the solvent is recovered and recycled.

In a belt dryer, the liquid is spread or sprayed onto a belt which passes over several heated plates underneath the belt. The material is heated by steam-heated or electrically heated plates. The evaporation of the solvent can additionally be fostered by infrared radiators or microwave radiators located over the belt. Belt drying may be carried out in a vacuum chamber.

In tray drying, the liquid mixture (or a dispersion product that has been pre-dried by any other method) is distributed over a number of trays. These are placed in an oven, usually in a stream of hot gas, e.g. air. Vacuum may be applied additionally.

The dried solid dispersion product may then be grinded and/or classified (sieved).

The dried solid dispersion product may then be filled into capsules or may be compacted. Compacting means a process whereby a powder mass comprising the solid dispersion product is densified under high pressure in order to obtain a compact with low porosity, e.g. a tablet. Compression of the powder mass is usually done in a tablet press, more specifically in a steel die between two moving punches.

At least one additive selected from flow regulators, disintegrants, bulking agents and lubricants is preferably used in compacting the granules. Disintegrants promote a rapid disintegration of the compact in the stomach and keep the liberated granules separate from one another. Suitable disintegrants are crosslinked polymers such as crosslinked polyvinyl pyrrolidone and crosslinked sodium carboxymethyl cellulose. Suitable bulking agents are selected from lactose, calcium hydrogenphosphate, microcrystalline cellulose (Avicel®), magnesium oxide, natural or pre-gelatinized potato or corn starch, polyvinyl alcohol.

Suitable flow regulators are selected from highly dispersed silica (Aerosil®), and animal or vegetable fats or waxes.

A lubricant is preferably used in compacting the granules. Suitable lubricants are selected from polyethylene glycol (e.g., having a Mw of from 1000 to 6000), magnesium and calcium stearates, sodium stearyl fumarate, talc, and the like.

Various other additives may be used, for example dyes such as azo dyes, organic or inorganic pigments such as aluminium oxide or titanium dioxide, or dyes of natural origin; stabilizers such as antioxidants, light stabilizers, radical scavengers, or stabilizers against microbial attack.

In order to facilitate the intake of such a dosage form by a mammal, it is advantageous to give the dosage form an appropriate shape. Large tablets that can be swallowed comfortably are therefore preferably elongated rather than round in shape.

A film coat on the tablet further contributes to the ease with which it can be swallowed. A film coat also improves taste and provides an elegant appearance. If desired, the film coat may be an enteric coat. The film coat usually includes a polymeric film-forming material such as hydroxypropyl methylcellulose, hydroxypropyl cellulose, and acrylate or methacrylate copolymers. Besides a film-forming polymer, the film coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than about 5% by weight of the dosage form.

The following examples in conjunction with the above-described figures will serve to further illustrate the invention without limiting it.

### EXAMPLES

ABT 102 was received from Abbott Laboratories, Illinois, U.S.A. Other active agents were prepared as described below.

### A. Preparation of Active Agents

### Example 1

### N-[(4R)-6-fluoro-2,2-dimethyl-3,4-dihydro-2H-chromen-4-yl]-N′-(1-methyl-1H-indazol-4-yl)urea

### Example 1A

### 6-Fluoro-2,2-dimethylchroman-4-one

In a 500 mL round-bottomed flask was added 1-(5-fluoro-2-hydroxyphenyl)ethanone (20.0 g, 130 mmol, Aldrich Chemical), propan-2-one (19.0 mL, 260 mmol), and pyrrolidine (21.5 mL, 260 mmol) in methanol (150 mL) to give a orange solution. The reaction mixture was stirred at ambient temperature for 48 h. The reaction mixture was poured into EtOAc (200 mL) and washed with 1 N HCl (50 mL), saturated NaHCO₃ (50 mL), and brine (50 mL). The organic portion was dried (Na₂SO₄), filtered, and concentrated to provide an orange residue which was purified by silica gel chromatography (gradient elution, 0-20% EtOAc/hexanes) to provide the title compound (14.2 g, 73.1 mmol, 56%) as a white solid. MS (DCl/NH₃) m/z 208 (M+NH₄)⁺.

### Example 1B

### (S)-6-Fluoro-2,2-dimethylchroman-4-ol

A solution of methyl tert-butylether (34 mL), (R)-diphenyl(pyrrolidin-2-yl)methanol (1.10 g, 4.35 mmol), and borane-N,N-diethylaniline complex (18.5 mL, 104 mmol) was heated to 45° C. and Example 1A (16.9 g, 87.0 mmol) in methyl tert-butylether (136 mL) was added over 75 min via addition funnel. After the addition, LCMS showed complete reaction. After 15 min of additional stirring at 45° C., the reaction mixture was cooled to 10° C. and treated with MeOH (85 mL) over 10 min, keeping the temperature ≦15° C. (H₂ evolution). After stirring for 30 min at ambient temperature, 2 N HCl (85 mL) was added and the reaction mixture was stirred for 10 min. Methyl tert-butylether (170 mL) was added and the reaction mixture was partitioned. The organic portion was washed with 2 N HCl (85 mL) and brine (35 mL). The aqueous extracts were back-extracted with methyl tert-butylether (85 mL). The combined organic portions were dried (Na₂SO₄), filtered, and concentrated, to provide Example 1B (17.4 g, 89.0 mmol). Analysis by analytical chiral HPLC (Chiralcel OJ 4.6×25 mm, 20% isopropanol/hexane, 23° C., 0.5 mL/min) showed 99% ee versus a racemic reference (pre-pared as described above using sodium borohydride as the reducing agent). MS (DCl/NH₃) m/z 197 (M+H)⁺.

### Example 1C

### (R)-6-Fluoro-2,2-dimethylchroman-4-amine

A mixture of Example 1B (17.1 g, 87.0 mmol) in THF (340 mL) was cooled to −30° C. followed by addition of methanesulfonic anhydride (16.7 mL, 131 mmol). N,N-Diisopropylethylamine (21.3 mL, 122 mmol) was slowly added (internal temperature ≦−24° C.) to the reaction mixture. After 30 min, ˜50% conversion was observed by LC/MS, thus the reaction mixture was warmed to −10° C. After 20 min, the reaction mixture was warmed further to 0° C. After 20 min, additional Ms₂O (3.00 g, 0.2 equiv) and N,N-diisopropylethylamine (2.8 mL, 0.2 equiv) were added and the reaction mixture was stirred for 20 min. At 0° C., additional N,N-diisopropylethylamine (1.40 mL, 0.1 equiv) was added, the reaction mixture was stirred for 10 min, then was cooled to −30° C. and treated with tetra-N-butylammonium azide (49.5 g, 174 mmol). The resulting slurry was allowed to slowly warm to ambient temperature overnight. After 14 h, methanol (85 mL) was added followed by 2 N NaOH (85 mL; slight exotherm to 27° C.). The reaction was stirred for 30 min, then diluted with MTBE (340 mL) and water (170 mL). The layers were separated and the organic layer was washed with water (85 mL), 2 N HCl (2×85 mL), water (85 mL), and brine (34 mL). The acidic washes were back-extracted with MTBE (85 mL). The combined organic portions were dried (Na₂SO₄), filtered, and concentrated to give a yellow residue that was used without further purification.

The crude azide product above was suspended in THF (305 mL) and water (34 mL) and treated with triphenylphosphine (25.1 g, 96.0 mmol). The yellow solution was heated to 60° C. for 2.5 h. The reaction mixture was cooled and concentrated to remove THF. Dichloromethane (170 mL), 2 N HCl (85 mL), and water (425 mL) were added to form a homogeneous biphasic mixture. The layers were partitioned and the aqueous portion was washed with dichloromethane (85 mL). 2 N NaOH (100 mL) was added to the aqueous layer which was then extracted with dichlormethane (5×85 mL), dried (Na₂SO₄), filtered, and concentrated to give the title compound (12.6 g, 64.3 mmol, 74%). Analytical chiral HPLC (Chiralcel OJ 4.6×25 mm, 20% isopropanol/hexane, 23° C., 0.5 mL/min) showed 91% ee versus a racemic reference standard. MS (DCl/NH₃) m/z 196 (M+H)⁺.

### Example 1D

### (R)-6-Fluoro-2,2-dimethylchroman-4-amine, (R)-2-hydroxy-2-phenylacetic acid salt

Example 1C (12.6 g, 64.3 mmol) and isopropanol (126 mL) were heated to 50° C. while (R)-(−)-mandelic acid (9.79 g, 64.3 mmol) was added. At 43° C., solids were observed, and heating continued was up to 50° C. The mixture was aged at 50° C. for 10 min, then hexanes (126 mL) were added over 45 min at 50° C. Following the addition, the reaction mixture was cooled gradually to ambient temperature over 90 min, precipitated solids were filtered, and were washed with 1:1 isopropol-hexanes. The solid was dried in an oven at 45° C. overnight with air bleed, to give the title compound (17.2 g, 49.5 mmol, 77%) as a crystalline white solid. The solid had no detectable minor isomer by Analytical chiral HPLC (Chiralcel OJ 4.6×25 mm, 20% isopropanol/hexane, 0.5 mL/min) and the mother liquor showed ˜50% ee in favor of the desired isomer. ¹H NMR (300 MHz, DMSO-d₆) δ7.44-7.37 (m, 3H), 7.30-7.17 (m, 3H), 7.01 (td, J=8.5, 3.1 Hz, 1H), 6.78-6.73 (m, 1H), 4.70 (s, 1H), 4.21 (dd, J=11.5, 6.3 Hz, 1H), 2.13 (dd, J=13.2, 6.3 Hz, 1H), 1.65 (t, J=12.3 Hz, 1H), 1.37 (s, 3H), 1.17 (s, 3H); MS (DCl/NH₃) m/z 179 (M−16)⁺.

### Example 1E

### 2-Bromo-6-fluorobenzaldehyde

1-Bromo-3-fluorobenzene (17.3 g, 100 mmol) was added over 5 min to a solution of lithium diisopropylamide (prepared from the addition of 40 mL of 2.5 N-butyllithium in hexanes to 11.5 g of 0.1 M diisopropylamine at 0° C.) in THF at −70° C. The mixture was stirred cold for 1 h, after which DMF (8 mL) was added over 10 min. The mixture was stirred at −70° C. for an additional 40 min, then was treat with acetic acid (26 g). The mixture was allowed to warm to ambient temperature, transferred into a mixture of MTBE (200 mL), water (200 mL), and 4 N hydrochloric acid (150 mL). The layers were partitioned and the organic portion was concentrated under reduced pressure to provide the title compound. MS (DCl/NH₃) m/z 202 (M+H)⁺.

### Example 1F

### 4-Bromo-1-methyl-1H-indazole

A solution of Example 1E (2.00 g, 9.95 mmol) in DMSO (3.5 mL) was added to methylhydrazine (98%, 3.20 g of 98% reagent, 69.6 mmol). The mixture was heated at 85° C. for 24 h, then cooled to ambient temperature and diluted with water (50 mL). The solution was extracted with CH₂Cl₂ (2×50 mL) and the combined organic layers were dried (MgSO₄), filtered, and concentrated under reduced pressure to provide the title compound which was used without further purification. MS (DCl/NH₃) m/z 202 (M+H)⁺.

### Example 1G

### 1-Methyl-1H-indazol-3-amine

A mixture of palladium(II) acetate (82 mg, 2 mol %) and Xantphos (287 mg, 3 mol %) in toluene (10 mL) was stirred for 5 min at ambient temperature. To the solution was added a solution of Example 1F (3.68 g, 17.4 mmol) and benzophenone imine (3.00 g, 17.4 mmol) in toluene (30 mL). The mixture was evacuated and purged with nitrogen two times, then stirred at ambient temperature for 15 min. Sodium tert-butoxide (1.90 g, 24.4 mmol) was added and the mixture was evacuated and purged with nitrogen. The mixture was heated to between 80 and 85° C. for 2 h, cooled to ambient temperature, and diluted with water (30 mL). The layers were partitioned and the aqueous layer was extracted with additional toluene (20 mL). The combined organic layers were stirred with 6 N HCl (10 mL) for 1 h, then 40 mL of water was added to dissolve the solids. The toluene layer was discarded and aqueous layer filtered to remove insoluble material. The aqueous layer was adjusted to pH 14 with the addition of 50% NaOH and the resulting solid was filtered and dried to provide the title compound. MS (DCl/NH₃) m/z 202 (M+H)⁺.

### Example 1H

### N-[(4R)-6-Fluoro-2,2-dimethyl-3,4-dihydro-2H-chromen-4-yl]-N′-(1-methyl-1H-indazol-4-yl)urea

To a 100 mL round-bottomed flask was added N,N′-disuccinyl carbonate (1.38 g, 5.38 mmol), pyridine (0.435 mL, 5.38 mmol) and Example 1G (0.754 g, 5.12 mmol) in acetonitrile (15 mL). The brown solution was stirred at room temperature for 30 min and treated with a solution of Example 1D (1.00 g, 5.12 mmol) in acetonitrile (10 mL) followed by N,N-diisopropylethylamine (2.66 mL, 15.4 mmol). The reaction was stirred for 1 h, then poured into EtOAc (200 mL) and washed with saturated NaHCO₃ (50 mL) and 1 N HCl (50 mL). The solution was dried (Na₂SO₄), filtered, and concentrated. The resulting residue was purified by silica gel chromatography (gradient elution, 0-50% EtOAc/hexanes) to provide the title compound (1.54 g, 4.18 mmol, 82%) as an off-white solid. ¹H NMR (300 MHz, DMSO-d₆) δ 8.76 (s, 1H), 8.05 (d, J=0.9 Hz, 1H), 7.70 (dd, J=7.5, 0.7 Hz, 1H), 7.27 (d, J=7.7 Hz, 1H), 7.18 (dt, J=8.3, 0.8 Hz, 1H), 7.09 (ddd, J=9.4, 3.1, 0.9 Hz, 1H), 7.05-6.97 (m, 1H), 6.78 (dd, J=8.8, 4.8 Hz, 2H), 5.03-4.94 (m, 1H), 4.01 (s, 3H), 2.29-2.16 (m, 1H), 1.77 (dd, J=13.2, 10.9 Hz, 1H), 1.40 (s, 3H), 1.29 (s, 3H); MS (DCl/NH₃) m/z 369 (M+H)⁺.

### Example 2

### N-[(4R)-6-Fluoro-2,2-dimethyl-3,4-dihydro-2H-chromen-4-yl]-N′-1H-indazol-4-ylurea

### Example 2A

### 4-Nitro-1H-indazole

2-Methyl-3-nitroaniline (20.0 g, 131 mmol) in acetic acid (200 mL) was treated with NaNO₂ (20.0 g, 289 mmol) in water (50 mL) at 4° C. (mechanical stirring). The reaction mixture was allowed to warm to ambient temperature and was stirred for 16 h. Solvent was removed under reduced pressure, and the residue was treated with water (700 mL), and filtered. The filtered solid was dried at 45° C. in a vacuum oven for 10 h to provide the title compound which was used without further purification.

Alternatively, a 4-necked 5-L jacketed round bottom flask fitted with a mechanical stirrer and a thermocouple was charged with 2-methyl-3-nitroaniline (100 g, 658 mmol) and acetic acid (2000 mL). The solution was cooled to 14° C. and treated with a chilled (˜1° C.; ice-water bath) solution of NaNO₂ (100 g, 1450 mmol) in water (250 mL) added in one portion. The internal temperature rose from 14° C. to 28° C. over 5 min and remained at this temperature for 5 min. before gradually cooling to 15° C. The mixture was stirred for 24 h after and was then concentrated under reduced pressure to an approximate volume of 500 mL. The residue was resuspended in water (1800 mL) at ambient temperature for 21 h. The resulting orange solid was filtered, washed with water (3×250 mL), and dried in a vacuum oven at 70° C. to afford 97.0 g of the title compound as a bright orange solid which was used without further purification.

### Example 2B

### Methyl 4-nitro-1H-indazole-1-carboxylate

NaH (300 mg, 12.5 mmol) in N,N-dimethylformamide (5 mL) was treated with Example 2A (1.33 g, 10.0 mmol) at 0° C. The reaction mixture was allowed to warm to ambient temperature and stir for 1 h. The mixture was then treated with methyl chloroformate (0.90 mL) and stirred at room temperature for 3 h. The reaction was quenched with water and filtered to provide the title compound as an off white solid.

Alternatively, to a 3-necked 2-L jacketed flask fitted with a mechanical stirrer, a thermocouple, and an addition funnel was charged with Example 2A (95.2 g, 716 mmol) and N,N-dimethylformamide (650 mL). The dark solution was cooled to 10° C. and DBU (96.0 g, 788 mmol.) was added via addition funnel so that the internal temperature did not go beyond 15° C. After cooling the mixture back to 10° C., methyl chloroformate (108 g, 1430 mmol) was added via addition funnel so that the internal temperature did not go beyond 25° C. After 1 h of stirring at 10° C., aqueous 10% potassium phosphate diacid in water (500 mL) was added and the mixture was stirred for 15 h. The resulting brown solid was filtered and the reaction mixture vessel rinsed with aqueous 10% potassium phosphate diacid in water (2×150 mL). The rinses were added to the solid on the filter. The resulting solid was washed with aqueous 10% potassium phosphate diacid in water (2×200 mL) and water (2×200 mL), then was dried in a vacuum oven at 70° C. to afford 122 g of a dark brown solid. The solid was resuspended in isopropyl acetate (2000 mL) for 2 h. The solid was filtered, washed with fresh isopropyl acetate (2×250 mL), and dried in a vacuum oven at 70° C. to afford the title compound (110 g, 495 mmol) as a light brown solid. MS (DCl/NH₃) m/z 222 (M+H)⁺.

### Example 2C

### Methyl 4-amino-1H-indazole-1-carboxylate

Example 2B (1.66 g, 7.50 mmol) and 10% Pd/C were combined in ethanol (20 mL) and exposed to hydrogen gas (1 atm pressure). The reaction mixture was heated at 80° C. for 20 min, allowed to cool to ambient temperature, and filtered through Celite. The filtrate was evaporated to provide title compound (1.22 g, 6.35 mmol). MS (DCl/NH₃) m/z 192 (M+H)⁺.

### Example 2D

### N-[(4R)-6-Fluoro-2,2-dimethyl-3,4-dihydro-2H-chromen-4-yl]-N′-1H-indazol-4-ylurea

To a 100 mL round-bottomed flask was added N,N′-disuccinyl carbonate (1.38 g, 5.38 mmol), pyridine (0.435 mL, 5.38 mmol) and Example 2C (983 mg, 5.12 mmol) in acetonitrile (15 mL). The brown solution was stirred at room temperature for 30 min and the treated with a solution of Example 1D (1.00 g, 5.12 mmol) in acetonitrile (10 mL) followed by N,N-diisopropylethylamine (2.66 mL, 15.4 mmol). The reaction was stirred for 1 h, then poured into ethyl acetate (200 mL) and washed with saturated NaHCO₃ (50 mL) and 1 N HCl (50 mL). The solution was dried (Na₂SO₄), filtered, and concentrated.

The resulting residue was dissolved in tetrahydrofuran (15 mL) and MeOH (15 mL) to give a yellow solution. To the solution was added 5N NaOH (4.8 mL) and the reaction mixture was stirred at ambient temperature for 2 h. The reaction mixture was poured into EtOAc (200 mL) and washed with saturated sodium bicarbonate (50 mL). The organic portion was dried (Na₂SO₄), filtered, and concentrated. Purified on by silica gel chromatography (gradient elution, with 0-10% MeOH/CH₂Cl₂) provided the title compound (1.10 g, 3.11 mmol, 83%) as a white amorphous solid. ¹H NMR (300 MHz, DMSO-d₆) δ 13.06-13.04 (br s, 1H), 8.76 (s, 1H), 8.08 (t, J=1.1 Hz, 1H), 7.68 (d, J=7.2 Hz, 1H), 7.23 (d, J=7.76 Hz, 1H), 7.11-6.98 (m, 3H), 6.81-6.76 (m, 2H), 5.04-4.94 (m, 1H), 2.19 (dd, J=13.2, 6.2 Hz, 1H), 1.77 (dd, J=13.2, 10.9 Hz, 1H), 1.40 (s, 3H), 1.29 (s, 3H). MS (DCl/NH₃) m/z 355 (M+H)⁺; [α]²³_{D}=+ 39.2 (c 1.0, MeOH).

### Example 3

### N-[(4R)-6-Fluoro-2,2-dimethyl-31-3,4-dihydro-2H-chromen-4-yl]-N′-[(7S)-7-hydroxy-5,6,7,8-tetrahydronaphthalen-1-yl]urea

### Example 3A

### 8-Amino-1,2,3,4-tetrahydronaphthalen-2-ol

Ethanol (1 L) was added to 8-amino-2-naphthol (100 g, 610 mmol), Raney nickel (40 g, water wet), and sodium hydroxide (4.00 g, 8 mol % aqueous) in a stirred reactor. The reactor was sealed and sparged with hydrogen. The reaction mixture was stirred for 13 h at 85° C. and then an additional 8 h at 100° C. The mixture was then filtered through a pad of Celite. The resulting solution was treated with Darco G-60 (35 g) and heated to reflux for 1 h, then cooled to ambient temperature and stirred an additional 3 h. This mixture was filtered through Celite (350 g), and the pad washed with EtOAc (1.5 L). The solvent was removed in vacuo and methyl tert-butyl ether (1 L) was added. This was heated for 15 min at 50° C., stirred for 1 h at ambient temperature, filtered, and the solvent removed in vacuo. Approximately half of the resulting crude solid was purified by chromatography on silica gel (gradient elution, 2-30% MeOH/CH₂Cl₂) to give 37 g of the title compound as a light brown solid. ¹H NMR (300 MHz, CDCl₃) δ 6.96 (t, J=7.6 Hz, 1H), 6.55 (dd, J=10.7, 7.6 Hz, 2H), 4.44-4.24 (m, 1H), 2.95-2.80 (m, 3H), 2.38 (dd, J=16.1, 7.6 Hz, 1H), 2.09-1.96 (m, 1H), 1.85-1.70 (m, 1H).

### Example 3B

### (2S)-8-Amino-1,2,3,4-tetrahydronaphthalen-2-ol

Example 3A was dissolved in isopropanol, loaded on a Chiralpak IC chiral HPLC column (30 cm ID×250 cm), and eluted with 32% isopropanol/hexane at 25° C. with a flow rate of 20 mL/min. The earlier eluting peak (retention time=16 min) was collected and the solvent evaporated to afford the title compound as an off-white solid in 99.2% ee. MS (DCl/NH₃) m/z 164 (M+H)⁺, 181 (M+NH₄)⁺.

### Example 3C

### N-[(4R)-6-Fluoro-2,2-dimethyl-3,4-dihydro-2H-chromen-4-yl]-N′-[(7S)-7-hydroxy-5,6,7,8-tetrahydronaphthalen-1-yl]urea

To a suspension of di(N-succinimidyl) carbonate (703 mg, 2.75 mmol) in acetonitrile (5 mL) was added Example 3B (427 mg, 2.62 mmol) dissolved in acetonitrile (10 mL) and pyridine (0.222 mL, 2.75 mmol). The reaction was stirred for 20 min whereupon Example 1C (510.6 mg, 2.62 mmol) in acetonitrile (10 mL) and N,N-diisopropylethylamine (1.37 mL, 7.85 mmol) was added. The reaction was stirred for 16 h at ambient temperature. EtOAc (200 mL) was added and the reaction mixture was washed with water (2×200 mL) and brine (200 mL), and partitioned. The organic portion was dried (Na₂SO₄) and filtered. Solvent was evaporated under reduced pressure and a white solid precipitated from solution. The solid was collected, triturated with diethyl ether, and filtered. The solid was rinsed with diethyl ether, then hexanes, and air-dried to provide the title compound (737 mg, 1.92 mmol, 73% yield) as a beige powder. ¹H NMR (300 MHz, DMSO-d₆) δ 7.70 (d, J=7.9 Hz, 1H), 7.60 (s, 1H), 7.08-6.94 (m, 4H), 6.81-6.71 (m, 2H), 4.93 (dd, J=18.0, 7.2 Hz 1H), 4.86 (d, J=4.2 Hz, 1H), 3.98-3.87 (m, 1H), 2.91-2.63 (m, 3H), 2.37 (dd, J=16.5, 7.7 Hz, 1H), 2.15 (dd, J=13.2, 6.2 Hz, 1H), 1.93-1.83 (m, 1H), 1.69 (dd, J=13.0, 11.1 Hz, 1H), 1.63-1.52 (m, 1H), 1.39 (s, 3H), 1.26 (s, 3H); MS (ESI) m/z 385 (M+H)⁺; [α]²³_{D}=+ 38.02 (c 1.0, CH₃OH).

### Example 4

### N-[(4R)-6-Fluoro-2,2-dimethyl-3,4-dihydro-2H-chromen-4-yl]-N′-[(7R)-7-hydroxy-5,6,7,8-tetrahydronaphthalen-1-yl]urea

### Example 4A

### (2R)-8-Amino-1,2,3,4-tetrahydronaphthalen-2-ol

Example 3A was dissolved in isopropanol, loaded on a Chiralpak IC chiral HPLC column (30 cm ID×250 cm), and eluted with 32% isopropanol/hexane at 25° C. with a flow rate of 20 mL/min. The later eluting peak (retention time=19 min) was collected and the solvent evaporated to afford the title compound as an off-white solid in 99.6% ee. MS (DCl/NH₃) m/z 164 (M+H)⁺, 181 (M+NH₄)⁺.

### Example 4B

### N-[(4R)-6-Fluoro-2,2-dimethyl-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B. ¹H NMR (300 MHz, DMSO-d₆) δ 7.69 (d, J=7.9 Hz, 1H), 7.61 (s, 1H), 7.08-6.94 (m, 4H), 6.81-6.71 (m, 2H), 4.99-4.88 (m, 1H), 4.86 (d, J=4.1 Hz, 1H), 4.00-3.88 (m, 1H), 2.90-2.64 (m, 3H), 2.35 (dd, J=16.5, 7.7 Hz, 1H), 2.15 (dd, J=13.2, 6.2 Hz, 1H), 1.93-1.81 (m, 1H), 1.69 (dd, J=13.0, 11.1 Hz, 1H), 1.64-1.51 (m, 1H), 1.39 (s, 3H), 1.27 (s, 3H); MS (DCl/NH₃) m/z 385 (M+H)⁺; [α]²³_{D}=+ 34.6° (c 1.0, CH₃OH).

### Example 5

### N-[(4R)-6-Fluoro-2,2-dimethyl-3,4-dihydro-2H-chromen-4-yl]-N′-isoquinolin-5-ylurea

In a 500 mL round-bottomed flask was added N,N′-disuccinimidyl carbonate (1.38 g, 5.38 mmol), pyridine (0.435 mL, 5.38 mmol) and isoquinolin-5-amine (0.738 g, 5.12 mmol, Acros) in acetonitrile (15 mL) to give a brown solution. The reaction was stirred at ambient temperature for 30 min. To the mixture was added Example 1C (1.00 g, 5.12 mmol) in acetonitrile (10 mL) and N,N-diisopropylethylamine (2.66 mL, 154 mmol). The reaction was stirred for 90 min then was concentrated. The mixture was diluted with EtOAc (300 mL) and was washed with saturated NaHCO₃ (100 mL) dried (Na₂SO₄), filtered and concentrated. The residue was purified by silica gel chromatography (gradient elution, 0-10% MeOH/CH₂Cl₂) to give the title compound (1.12 g, 3.07 mmol, 60%) as a white solid. ¹H NMR (300 MHz, DMSO-d₆) δ 9.29 (d, J=0.8 Hz, 1H), 8.76 (s, 1H), 8.56 (d, J=6.0 Hz, 1H), 8.34 (dd, J=7.7, 1.1 Hz, 1H), 7.94 (d, J=6.1 Hz, 1H), 7.77 (d, J=8.1 Hz, 1H), 7.64 (t, J=7.9 Hz, 1H), 7.12 (ddd, J=9.4, 3.2, 0.9 Hz, 1H), 7.06-6.98 (m, 2H), 6.79 (dd, J=8.9, 4.9 Hz, 1H), 5.05-4.95 (m, 1H), 2.21 (dd, J=13.2, 6.2 Hz, 1H), 1.78 (dd, J=13.2, 10.9 Hz, 1H), 1.41 (s, 3H), 1.29 (s, 3H); MS (DCl/NH₃) m/z 366 (M+H)⁺; [α]²³_{D}=+ 32.6 (c 0.65, CH₃OH).

### Example 6

### N-[(4R)-6-Fluoro-3,3′,4,4′-tetrahydro-2′ H-spiro[chromene-2,1′-cyclobutan]-4-yl]-N′-[(7R)-7-hydroxy-5,6,7,8-tetrahydronaphthalen-1-yl]urea

### Example 6A

### 6-Fluorospiro[chroman-2,1′-cyclobutan]-4-one

The title compound was prepared according to the procedure of Example 1A, using 1-(5-fluoro-2-hydroxyphenyl)ethanone and substituting cyclobutanone for propan-2-one. MS (DCl/NH₃) m/z 207 (M+H)⁺.

### Example 6B

### (E)-6-Fluorospiro[chroman-2,1′-cyclobutan]-4-one O-methyl oxime

In a 500 mL round-bottomed flask was added Example 6A (19.4 g, 94.9 mmol) and O-methylhydroxylamine hydrochloride (8.53 mL, 112 mmol) in pyridine (150 mL) to give a yellow solution. The reaction mixture was stirred for 54 h at ambient temperature, concentrated, diluted with EtOAc (1 L), and washed with water (400 mL). The organic portion was dried (Na₂SO₄), filtered and concentrated. The resulting yellow residue was purified by silica gel chromatography (gradient elution, 0-30% EtOAc/hexanes) to provide the title compound (21.8 g, 94.0 mmol, 99%) as a pale yellow solid. MS (DCl/NH₃) m/z 224 (M+NH₄)⁺.

### Example 6C

### 6-Fluorospiro[chroman-2,1′-cyclobutan]-4-amine

Example 6B (21.8 g, 94.0 mmol) and Raney nickel (5.49 g, water wet) were stirred in EtOH containing 7 M ammonia (150 mL). The reactor was sealed and sparged with hydrogen. The reaction mixture was stirred for 3 h at 32° C., cooled, diluted with EtOAc (250 mL) and filtered through a pad of Celite (50 g). The resulting solution was filtered through a plug of silica gel (50 g) and the filtrate evaporated to give the title compound (10.8 g, 52.1 mmol, 56%) as a pale oil. MS (DCl/NH₃) m/z 208 (M+H)⁺.

### Example 6D

### (R)-6-Fluorospiro[chroman-2,1′-cyclobutan]-4-amine

Example 6C was resolved by semi-preparative chiral HPLC (Chiralcel OD 5×50 cm, 5% isopropanol/hexane+0.1% diethylamine, 23° C., 100 mL/min). The later of the two eluting peaks (retention time=26.0 min) was collected and the solvent evaporated to afford the title compound as an off-white solid in 99% ee versus a racemic reference (prepared as described above using sodium borohydride as the reducing agent). MS (DCl/NH₃) m/z 208 (M+H)⁺.

### Example 6E

### (R)-6-Fluorospiro[chroman-2,1′-cyclobutan]-4-amine (R)-2-hydroxy-2-phenylacetic acid salt

The title compound was prepared according to the procedure of Example 1D, substituting Example 6D for Example 1C. MS (DCl/NH₃) m/z 208 (M+H)⁺.

### Example 6F

### N-[(4R)-6-Fluoro-3,3′,4,4′-tetrahydro-2′H-spiro[chromene-2,1′-cyclobutan]-4-yl]-N′-[(7R)-7-hydroxy-5,6,7,8-tetrahydronaphthalen-1-yl]urea

The title compound was prepared according to the procedure of Example 3C, substituting Example 6E for Example 1D, and substituting Example 4A for Example 3B. ¹H NMR (300 MHz, DMSO-d₆) δ 7.70 (d, J=7.8 Hz, 1H), 7.62 (s, 1H), 7.06-6.96 (m, 4H), 6.81 (dd, J=9.6, 4.9 Hz, 1H), 6.74 (d, J=7.4 Hz, 1H), 4.93 (dd, J=14.8, 9.1 Hz 1H), 4.86 (d, J=4.1 Hz, 1H), 3.99-3.88 (m, 1H), 2.91-2.64 (m, 3H), 2.42-2.03 (m, 6H), 1.93-1.67 (m, 4H), 1.67-1.52 (m, 1H); MS (ESI) m/z 397 (M+H)⁺; [α]²³_{D}=+ 62.8° (c 1.0, CH₃OH)

### Example 7

### N-[(4R)-6-Fluoro-3,3′,4,4′-tetrahydro-2′H-spiro[chromene-2,1′-cyclobutan]-4-yl]-N′-(1-methyl-1H-indazol-4-yl)urea

The title compound was prepared according to the procedure of Example 1H, substituting Example 6E for Example 1D. ¹H NMR (300 MHz, DMSO-d₆) δ 8.79 (s, 1H), 8.05 (d, J=0.9 Hz, 1H), 7.72 (dd, J=7.5, 0.7 Hz, 1H), 7.28 (d, J=7.7 Hz, 1H), 7.20-7.16 (m, 1H), 7.09-6.99 (m, 2H), 6.83 (dd, J=8.7, 4.7 Hz, 2H), 5.03-4.94 (m, 1H), 4.01 (s, 3H), 2.51-2.38 (m, 1H), 2.36-2.04 (m, 4H), 2.00-1.68 (m, 3H); MS (DCl/NH₃) m/z 381 (M+H)⁺; [α]^{D}₂₃=+34.45 (c 0.50, CH₃OH).

### Example 8

### N-[(4R)-6-Fluoro-3,3′,4,4′-tetrahydro-2′H-spiro[chromene-2,1′-cyclobutan]-4-yl]-N′-1H-indazol-4-ylurea

The title compound was prepared according to the procedure of Example 2D, substituting Example 6E for Example 1D. ¹H NMR (300 MHz, DMSO-d₆) δ 13.03-13.01 (br s, 1H), 8.75 (s, 1H), 8.08 (s, 1H), 7.68 (d, J=7.2 Hz, 1H), 7.22 (d, J=7.8 Hz, 1H), 7.11-6.94 (m, 3H), 6.86-6.81 (m, 2H), 5.03-4.94 (m, 1H), 2.45-2.06 (m, 5H), 1.95-1.69 (m, 3H); MS (DCl/NH₃) m/z 367 (M+H)⁺; [α]^{D}₂₃=+24.1 (c 0.70, CH₃OH).

### Example 9

### N-[(4R)-6-Fluoro-3,3′,4,4′-tetrahydro-2′H-spiro[chromene-2,1′-cyclobutan]-4-yl]-N′-[(7S)-7-hydroxy-5,6,7,8-tetrahydronaphthalen-1-yl]urea

The title compound was prepared according to the procedure of Example 3C, substituting Example 6E for Example 1D. ¹H NMR (300 MHz, DMSO-d₆) δ 7.71 (d, J=7.3 Hz, 1H), 7.61 (s, 1H), 7.07-6.95 (m, 4H), 6.86-6.77 (m, 1H), 6.74 (d, J=7.4 Hz, 1H), 4.92 (dd, J=14.5, 9.2 Hz, 1H), 4.85 (d, J=4.3 Hz, 1H), 3.99-3.87 (m, 1H), 2.91-2.64 (m, 3H), 2.42-2.03 (m, 6H), 1.93-1.67 (m, 4H), 1.67-1.52 (m, 1H); MS (ESI) m/z 397 (M+H)⁺; [α]²³_{D}=+ 68.4° (c 1.0, CH₃OH).

### Example 10

### N-[(4S)-6-Fluoro-3,3′,4,4′-tetrahydro-2′H-spiro[chromene-2,1′-cyclobutan]-4-yl]-N′-[(7S)-7-hydroxy-5,6,7,8-tetrahydronaphthalen-1-yl]urea

### Example 10A

### (S)-6-Fluorospiro[chroman-2,1′-cyclobutan]-4-amine

Example 6C was resolved by semi-preparative chiral HPLC (Chiralcel OD 5×50 cm, 5% isopropanol/hexane+0.1% diethylamine, 23° C., 100 mL/min). The earlier of the two eluting peaks (retention time=20.9 min) was collected and the solvent evaporated to afford the title compound as an off-white solid in 99% ee versus a racemic reference (prepared as described above using sodium borohydride as the reducing agent).

MS (DCl/NH₃) m/z 208 (M+H)⁺.

### Example 10B

### N-[(4S)-6-Fluoro-3,3′,4,4′-tetrahydro-2′H-spiro[chromene-2,1′-cyclobutan]-4-yl]-N′-[(7S)-7-hydroxy-5,6,7,8-tetrahydronaphthalen-1-yl]urea

The title compound was prepared according to the procedure of Example 3C, substituting Example 10A for Example 1D. ¹H NMR (300 MHz, DMSO-d₆) δ 7.71 (d, J=7.4 Hz, 1H), 7.62 (s, 1H), 7.07-6.96 (m, 4H), 6.81 (dd, J=9.6, 4.8 Hz, 1H), 6.74 (d, J=7.0 Hz, 1H), 4.98-4.89 (m, 1H), 4.87 (d, J=4.1 Hz, 1H), 3.99-3.89 (m, 1H), 2.90-2.64 (m, 3H), 2.41-2.02 (m, 6H), 1.92-1.67 (m, 4H), 1.66-1.51 (m, 1H); MS (ESI) m/z 397 (M+H)⁺; [α]²³_{D}=−59.5° (c 1.0, CH₃OH).

### Example 11

### N-[(4S)-6-Fluoro-3,3′,4,4′-tetrahydro-2′ H-spiro[chromene-2,1′-cyclobutan]-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 10A for Example 1D. ¹H NMR (300 MHz, DMSO-d₆) δ 7.71 (d, J=7.4 Hz, 1H), 7.61 (s, 1H), 7.06-6.95 (m, 4H), 6.85-6.77 (m, 1H), 6.74 (d, J=7.5 Hz, 1H), 4.92 (dd, J=15.0, 9.1 Hz, 1H), 4.85 (d, J=5.3 Hz, 1H), 3.99-3.87 (m, 1H), 2.90-2.64 (m, 3H), 2.43-2.03 (m, 6H), 1.92-1.66 (m, 4H), 1.66-1.52 (m, 1H); MS (ESI) m/z 397 (M+H)⁺; [α]²³_{D}=−63.0° (c 1.0, CH₃OH).

### Example 12

### N-[(4R)-6-Fluoro-3,4-dihydro-2H-chromen-4-yl]-N′-(1-methyl-1H-indazol-4-yl)urea

### Example 12A

### 6-Fluorochroman-4-one

The title compound was prepared according to the procedure of Example 1A, substituting paraformaldehyde for propan-2-one. MS (DCl/NH₃) m/z 183 (M+NH₄)⁺.

### Example 12B

### (R)-6-Fluorochroman-4-amine, (R)-2-hydroxy-2-phenylacetic acid salt

The title compound was prepared from Example 12A according to the methods described in Example 1B, Example 1C, and Example 1D. MS (DCl/NH₃⁺) m/z 168 (M+H)⁺.

### Example 12C

### N-[(4R)-6-Fluoro-3,4-dihydro-2H-chromen-4-yl]-N′-(1-methyl-1H-indazol-4-yl)urea

The title compound was prepared according to the procedure of Example 1H, substituting Example 12B for Example 1D. ¹H NMR (300 MHz, DMSO-d₆) δ 8.63 (s, 1H), 8.00 (d, J=0.9 Hz, 1H), 7.70 (d, J=7.5 Hz, 1H), 7.43-7.25 (m, 2H), 7.18-6.79 (m, 5H), 5.01-4.88 (m, 1H), 4.20-4.00 (m, 4H), 2.20-1.84 (m, 2H); MS (DCl/NH₃) m/z 341 (M+H)⁺; [α]²³_{D}=+ 37 (c 0.15, MeOH).

### Example 13

### N-[(4R)-6-Fluoro-3,4-dihydro-2H-chromen-4-yl]-N′-isoquinolin-5-ylurea

The title compound was prepared according to the procedure of Example 5, substituting Example 12B for Example 1C. ¹H NMR (300 MHz, DMSO-d₆) δ 9.28 (s, 1H), 8.62 (s, 1H), 8.54 (d, J=6.0 Hz, 1H), 8.36 (d, J=8.1 Hz, 1H), 7.90 (d, J=6.1 Hz, 1H), 7.76 (d, J=8.1 Hz, 1H), 7.63 (t, J=7.9 Hz, 1H), 7.24-6.99 (m, 3H), 6.85 (dd, J=9.0, 4.9 Hz, 1H), 4.97-4.90 (m, 1H), 4.33-4.23 (m, 1H), 4.18 (ddd, J=11.3, 8.3, 3.0 Hz, 1H), 2.28-1.96 (m, 2H); MS (DCl/NH₃) m/z 338 (M+H)⁺; [α]²³_{D}=+ 29.0 (c 0.25 CH₃OH).

### Example 14

### N-[(4R)-6-Fluoro-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 12B for Example 1D. ¹H NMR (300 MHz, DMSO-d₆) δ 7.73 (d, J=8.0 Hz, 1H), 7.49 (s, 1H), 7.13 (d, J=7.7 Hz, 1H), 7.10-6.96 (m, 3H), 6.82 (dd, J=8.9, 4.9 Hz, 1H), 6.72 (d, J=7.3 Hz, 1H), 4.89-4.82 (m, 2H), 4.26 (ddd, J=10.1, 6.8, 3.2 Hz, 1H), 4.13 (ddd, J=11.2, 8.4, 2.9 Hz, 1H), 3.98-3.87 (m, 1H), 2.88-2.62 (m, 3H), 2.31 (dd, J=16.8, 8.0 Hz, 1H), 2.18-2.04 (m, 1H), 2.01-1.81 (m, 2H), 1.66-1.50 (m, 1H); MS (ESI) m/z 357 (M+H)⁺; [α]²³_{D}=+ 66.12° (c 1.0, 1:1 DMSO:CH₃OH).

### Example 15

### N-[(4R)-6-Fluoro-3,4-dihydro-2H-chromen-4-yl]-N′-[(7S)-7-hydroxy-5,6,7,8-tetrahydronaphthalen-1-yl]urea

The title compound was prepared according to the procedure of Example 3C, substituting Example 12B for Example 1D. ¹H NMR (300 MHz, DMSO-d₆) δ 7.74 (d, J=8.0 Hz, 1H), 7.49 (s, 1H), 7.13 (d, J=7.7 Hz, 1H), 7.10-6.96 (m, 3H), 6.82 (dd, J=8.9, 4.9 Hz, 1H), 6.72 (d, J=7.5 Hz, 1H), 4.90-4.81 (m, 2H), 4.26 (ddd, J=10.2, 6.6, 3.1 Hz, 1H), 4.13 (ddd, J=11.3, 8.4, 2.9 Hz, 1H), 3.98-3.85 (m, 1H), 2.90-2.62 (m, 3H), 2.32 (dd, J=16.5, 7.7 Hz, 1H), 2.17-2.03 (m, 1H), 2.01-1.81 (m, 2H), 1.67-1.50 (m, 1H);

MS (ESI) m/z 357 (M+H)⁺; [α]²³_{D}=+ 62.02 (c 1.0, 1:1 DMSO:CH₃OH).

### Example 16

### N-[(4R)-6,8-Difluoro-2,2-dimethyl-3,4-dihydro-2H-chromen-4-yl]-N′-(1-methyl-1H-indazol-4-yl)urea

### Example 16A

### 6,8-Difluoro-2,2-dimethylchroman-4-one

The title compound was prepared according to the procedure of Example 1A, substituting 1-(3,5-difluoro-2-hydroxyphenyl)ethanone for 1-(5-fluoro-2-hydroxyphenyl)ethanone. MS (DCl/NH₃) m/z 230 (M+NH₄)⁺.

### Example 16B

### (R)-6,8-Difluoro-2,2-dimethylchroman-4-amine, (R)-2-hydroxy-2-phenylacetic acid salt

The title compound was prepared from Example 16A according to the methods described in Example 1B, Example 1C, and Example 1D. MS (DCl/NH₃) m/z 214 (M+H)⁺.

### Example 16C

### N-[(4R)-6,8-Difluoro-2,2-dimethyl-3,4-dihydro-2H-chromen-4-yl]-N′-(1-methyl-1H-indazol-4-yl)urea

The title compound was prepared according to the procedure of Example 1H, substituting Example 16B for Example 1D. ¹H NMR (300 MHz, DMSO-d₆) δ 8.80 (s, 1H), 8.05 (d, J=0.9 Hz, 1H), 7.69 (dd, J=7.5, 0.8 Hz, 1H), 7.32-7.15 (m, 3H), 6.99-6.94 (m, 1H), 6.80 (d, J=8.3 Hz, 1H), 5.06-4.96 (m, 1H), 4.01 (s, 3H), 2.23 (dd, J=13.3, 6.2 Hz, 1H), 2.00-1.81 (m, 1H), 1.45 (s, 3H), 1.32 (s, 3H); MS (DCl/NH₃) m/z 387 (M+H)⁺; [α]²³_{D}=+ 19.3 (c 0.73, MeOH).

### Example 17

### N-[(4R)-6,8-Difluoro-2,2-dimethyl-3,4-dihydro-2H-chromen-4-yl]-N′-isoquinolin-5-ylurea

The title compound was prepared according to the procedure of Example 5, substituting Example 16B for Example 1C. ¹H NMR (300 MHz, DMSO-d₆) δ 9.29 (d, J=0.8 Hz, 1H), 8.77 (s, 1H), 8.56 (d, J=6.0 Hz, 1H), 8.35 (dd, J=7.7, 1.1 Hz, 1H), 7.93 (d, J=6.1 Hz, 1H), 7.77 (d, J=8.1 Hz, 1H), 7.64 (t, J=7.9 Hz, 1H), 7.18-7.07 (m, 2H), 7.04 (d, J=8.4 Hz, 1H), 6.89 (td, J=7.9, 5.0 Hz, 1H), 5.10-5.01 (m, 1H), 2.24 (dd, J=13.3, 6.2 Hz, 1H), 2.00-1.81 (m, 1H), 1.46 (s, 3H), 1.33 (s, 3H). MS (DCl/NH₃) m/z 366 (M+H)⁺; [α]²³_{D}=+ 26.7 (c 0.70, CH₃OH).

### Example 18

### N-[(4R)-6,8-Difluoro-2,2-dimethyl-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 16B for Example 1D. ¹H NMR (300 MHz, DMSO-d₆) δ 7.71-7.61 (m, 2H), 7.17 (ddd, J=11.5, 8.8, 3.0 Hz, 1H), 7.06-6.97 (m, 2H), 6.91 (d, J=9.3 Hz, 1H), 6.75 (d, J=7.2 Hz, 1H), 5.02-4.89 (m, 1H), 4.86 (d, J=4.1 Hz, 1H), 4.00-3.87 (m, 1H), 2.90-2.64 (m, 3H), 2.35 (dd, J=16.5, 7.7 Hz, 1H), 2.19 (dd, J=13.3, 6.2 Hz, 1H), 1.93-1.82 (m, 1H), 1.77 (dd, J=13.2, 11.2 Hz, 1H), 1.68-1.51 (m, 1H), 1.43 (s, 3H), 1.30 (s, 3H); MS (ESI) m/z 403 (M+H)⁺;

[α]²³_{D}=+ 39-4° (c 1.0, CH₃OH).

### Example 19

### N-[(4R)-6,8-Difluoro-2,2-dimethyl-3,4-dihydro-2H-chromen-4-yl]-N′-[(7S)-7-hydroxy-5,6,7,8-tetrahydronaphthalen-1-yl]urea

The title compound was prepared according to the procedure of Example 3C, substituting Example 16B for Example 1D. ¹H NMR (300 MHz, DMSO-d₆) δ 7.72-7.62 (m, 2H), 7.17 (ddd, J=11.4, 8.7, 3.0 Hz, 1H), 7.06-6.97 (m, 2H), 6.91 (d, J=9.3 Hz, 1H), 6.75 (d, J=7.4 Hz, 1H), 5.02-4.88 (m, 1H), 4.86 (d, J=4.1 Hz, 1H), 3.98-3.86 (m, 1H), 2.91-2.61 (m, 3H), 2.37 (dd, J=16.5, 7.8 Hz, 1H), 2.19 (dd, J=13.3, 6.2 Hz, 1H), 1.93-1.82 (m, 1H), 1.77 (dd, J=13.2, 11.3 Hz, 1H), 1.67-1.52 (m, 1H), 1.43 (s, 3H), 1.30 (s, 3H); MS (ESI) m/z 403 (M+H)⁺; [α]²³_{D}=+ 42.8° (c 1.0, CH₃OH).

### Example 20

### N-[(4R)-8-Fluoro-2,2-dimethyl-3,4-dihydro-2H-chromen-4-yl]-N′-[(hydroxy-5,6,7,8-tetrahydronaphthalen-1-yl]urea

### Example 20A

### 8-Fluoro-2,2-dimethylchroman-4-one

The title compound was prepared according to the procedure of Example 1A, substituting 1-(3-fluoro-2-hydroxyphenyl)ethanone for 1-(5-fluoro-2-hydroxyphenyl)ethanone and using propan-2-one. MS (DCl/NH₃) m/z 212 (M+NH₄)⁺.

### Example 20B

### (R)-8-Fluoro-2,2-dimethylchroman-4-amine, (R)-2-hydroxy-2-phenylacetic acid salt

The title compound was prepared from Example 20A according to the methods described in Example 1B, Example 1C, and Example 1D. MS (DCl/NH₃) m/z 196 (M+H)⁺.

### Example 20C

### N-[(4R)-8-Fluoro-2,2-dimethyl-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 20B for Example 1D. ¹H NMR (300 MHz, DMSO-d₆) δ 7.70 (d, J=7.4 Hz, 1H), 7.60 (s, 1H), 7.14-6.94 (m, 4H), 6.87 (td, J=8.0, 5.0 Hz, 1H), 6.74 (d, J=7.1 Hz, 1H), 5.04-4.92 (m, 1H), 4.86 (d, J=4.2 Hz, 1H), 3.99-3.86 (m, 1H), 2.90-2.63 (m, 3H), 2.36 (dd, J=16.6, 7.8 Hz, 1H), 2.18 (dd, J=13.3, 6.2 Hz, 1H), 1.93-1.82 (m, 1H), 1.76 (dd, J=13.3, 10.9 Hz, 1H), 1.67-1.51 (m, 1H), 1.44 (s, 3H), 1.31 (s, 3H); MS (ESI) m/z 385 (M+H)⁺; [α]²³_{D}=+ 35.8° (c 1.0, CH₃OH).

### Example 21

### N-[(4R)-8-Fluoro-2,2-dimethyl-3,4-dihydro-2H-chromen-4-yl]-N′-[(7S)-7-hydroxy-5,6,7,8-tetrahydronaphthalen-1-yl]urea

The title compound was prepared according to the procedure of Example 3C, substituting Example 20B for Example 1D. ¹H NMR (300 MHz, DMSO-d₆) δ 7.69 (d, J=7.4 Hz, 1H), 7.60 (s, 1H), 7.14-6.94 (m, 2H), 6.87 (td, J=8.0, 5.0 Hz, 1H), 6.74 (d, J=7.3 Hz, 1H), 5.04-4.92 (m, 1H), 4.86 (d, J=4.2 Hz, 1H), 3.99-3.87 (m, 1H), 2.89-2.64 (m, 3H), 2.34 (dd, J=16.5, 7.8 Hz, 1H), 2.19 (dd, J=13.4, 6.2 Hz, 1H), 1.93-1.82 (m, 2H), 1.76 (dd, J=13.3, 11.0 Hz, 1H), 1.61 (d, J=5.4 Hz, 1H), 1.44 (s, 3H), 1.31 (s, 3H); MS (ESI) m/z 385 (M+H)⁺; [α]²³_{D}=+ 30.7° (c 1.0, CH₃OH).

### Example 22

### N-[(4R)-8-Fluoro-2,2-dimethyl-3,4-dihydro-2H-chromen-4-yl]-N′-isoquinolin-5-ylurea

The title compound was prepared according to the procedure of Example 5 substituting Example 20B for Example 1C. ¹H NMR (300 MHz, DMSO-d₆) δ 9.29 (d, J=0.9 Hz, 1H), 8.78 (s, 1H), 8.56 (d, J=6.0 Hz, 1H), 8.32 (dd, J=7.7, 1.1 Hz, 1H), 7.94 (d, J=6.1 Hz, 1H), 7.78 (d, J=8.1 Hz, 1H), 7.64 (t, J=7.9 Hz, 1H), 7.20 (ddd, J=11.3, 8.5, 2.9 Hz, 1H), 7.07-6.97 (m, 2H), 5.08-4.91 (m, 1H), 2.31-2.03 (m, 1H), 1.91-1.82 (m, 1H), 1.45 (s, 3H), 1.32 (s, 3H). MS (DCl/NH₃) m/z 384 (M+H)⁺; [α]²³_{D}=+ 32.5 (c 0.63, CH₃OH).

### Example 23

### N-[(4R)-7-Fluoro-2,2-dimethyl-3,4-dihydro-2H-chromen-4-yl]-N′-isoquinolin-5-ylurea

### Example 23A

### 7-Fluoro-2,2-dimethylchroman-4-one

The title compound was prepared according to the procedure of Example 1A, substituting 1-(4-fluoro-2-hydroxyphenyl)ethanone for 1-(5-fluoro-2-hydroxyphenyl)ethanone. MS (DCl/NH₃) m/z 212 (M+NH₄)⁺.

### Example 23B

### (R)-7-Fluoro-2,2-dimethylchroman-4-amine, (R)-2-hydroxy-2-phenylacetic acid salt

The title compound was prepared from Example 23A according to the methods described in Example 1B, Example 1C, and Example 1D. MS (DCl/NH₃) m/z 196 (M+H)⁺.

### Example 23C

### N-[(4R)-7-Fluoro-2,2-dimethyl-3,4-dihydro-2H-chromen-4-yl]-N′-isoquinolin-5-ylurea

The title compound was prepared according to the procedure of Example 5 substituting Example 23B for Example 1C. ¹H NMR (300 MHz, DMSO-d₆) δ 9.29 (s, 1H), 8.72 (s, 1H), 8.56 (d, J=6.0 Hz, 1H), 8.35 (d, J=7.7 Hz, 1H), 7.93 (d, J=6.1 Hz, 1H), 7.77 (d, J=8.1 Hz, 1H), 7.63 (t, J=7.9 Hz, 1H), 7.39-7.34 (m, 1H), 6.98 (d, J=8.3 Hz, 1H), 6.76 (td, J=8.5, 2.7 Hz, 1H), 6.62 (dd, J=10.6, 2.6 Hz, 1H), 5.05-4.95 (m, 1H), 2.21 (dd, J=13.3, 6.1 Hz, 1H), 1.79 (dd, J=13.2, 10.7 Hz, 1H), 1.42 (s, 3H), 1.32 (m, 3H). MS (DCl/NH₃) m/z 366 (M+H)⁺; [α]²³_{D}=+ 28.5 (c 0.82, CH₃OH).

### Example 24

### N-[(4R)-7-Fluoro-2,2-dimethyl-3,4-dihydro-2H-chromen-4-yl]-N′-(1-methyl-1H-indazol-4-yl)urea

The title compound was prepared according to the procedure of Example 1H, substituting Example 23B for Example 1D. ¹H NMR (300 MHz, DMSO-d₆) δ 8.74 (s, 1H), 8.04 (s, 1H), 7.71 (d, J=7.5 Hz, 1H), 7.37-7.25 (m, 2H), 7.17 (d, J=8.3 Hz, 1H), 6.76 (dd, J=8.6, 2.7 Hz, 1H), 6.72 (d, J=8.2 Hz, 1H), 6.61 (dd, J=10.6, 2.6 Hz, 1H), 5.03-4.93 (m, 1H), 4.01 (s, 3H), 2.20 (dd, J=13.3, 6.1 Hz, 1H), 2.00-1.73 (m, 1H), 1.42 (s, 3H), 1.31 (s, 3H); MS (DCl/NH₃) m/z 369 (M+H)⁺; [α]²³_{D}=+ 11 (c 0.61, CH₃OH).

### Example 25

### N-[(4R)-8-Fluoro-2,2-dimethyl-3,4-dihydro-2H-chromen-4-yl]-N′-(1-methyl-1H-indazol-4-yl)urea

The title compound was prepared according to the procedure of Example 1H, substituting Example 20B for Example 1D. ¹H NMR (300 MHz, DMSO-d₆) δ 8.76 (s, 1H), 8.04 (d, J=0.9 Hz, 1H), 7.70 (dd, J=7.5, 0.8 Hz, 1H), 7.27 (d, J=7.7 Hz, 1H), 7.19-7.06 (m, 3H), 6.88 (td, J=7.9, 5.0 Hz, 1H), 6.76 (d, J=8.4 Hz, 1H), 5.09-4.99 (m, 1H), 4.01 (s, 3H), 2.22 (dd, J=13.3, 6.2 Hz, 1H), 1.84 (dd, J=13.3, 10.8 Hz, 1H), 1.45 (s, 3H), 1.33 (s, 3H); MS (DCl/NH₃) m/z 369 (M+H)⁺; [α]²³_{D}=+ 13 (c 0.67, CH₃OH).

### Example 26

### N-[(4R)-2,2-Diethyl-6-fluoro-3,4-dihydro-2H-chromen-4-yl]-N′-(1-methyl-1H-indazol-4-yl)urea

### Example 26A

### 2,2-Diethyl-6-fluorochroman-4-one

1-(5-Fluoro-2-hydroxyphenyl)ethanone (30.2 g, 196 mmol) and MeOH (300 mL) were stirred at ambient temperature and 3-pentanone (41.6 mL, 392 mmol) and pyrrolidine (17.8 mL, 216 mmol) were added. The mixture was heated to 60° C. for 62 h at which point LCMS analysis showed clean conversion to product. The reaction was cooled, concentrated to a minimal volume of MeOH, and MTBE (300 mL) was added. The organics were washed with 2N HCl (150 mL), brine (60 mL), 2N NaOH (150 mL), and brine (60 mL). The solution was passed through a plug of silica gel (30 g), washing with MTBE (150 mL). The filtrate was concentrated, giving the title compound (38.8 g, 175 mmol, 89%) as a light brown oil. MS (DCl/NH₃) m/z 240 (M+NH₄)⁺.

### Example 26B

### (R)-6-Fluoro-2,2-diethylchroman-4-amine, (R)-2-hydroxy-2-phenylacetic acid salt

The title compound was prepared from Example 26A according to the methods described in Example 1B and Example 1C. MS (DCl/NH₃) m/z 224 (M+H)⁺.

### Example 26C

### N-[(4R)-2,2-Diethyl-6-fluoro-3,4-dihydro-2H-chromen-4-yl]-N′-(1-methyl-1H-indazol-4-yl)urea

The title compound was prepared according to the procedure of Example 1H, substituting Example 26B for Example 1D. ¹H NMR (300 MHz, DMSO-d₆) δ 8.75 (s, 1H), 8.05 (s, 1H), 7.70 (d, J=7.5 Hz, 1H), 7.28 (t, J=7.9 Hz, 1H), 7.18 (d, J=8.3 Hz, 1H), 7.09 (dd, J=9.4, 3.2 Hz, 1H), 7.01 (td, J=8.6, 3.2 Hz, 1H), 6.83-6.77 (m, 1H), 6.77 (d, J=8.2 Hz, 1H), 5.01-4.91 (m, 1H), 4.01 (s, 3H), 2.19 (dd, J=13.4, 6.1 Hz, 1H), 1.76-1.52 (m, 5H), 0.94-0.85 (m, 6H); MS (DCl/NH₃) m/z 397 (M+H)⁺; [α]²³_{D}=+ 9.2 (c 0.61, CH₃OH).

### Example 27

### N-[(4R)-2,2-Dimethyl-3,4-dihydro-2H-chromen-4-yl]-N′-isoquinolin-5-ylurea

### Example 27A

### 2,2-Dimethylchroman-4-one

The title compound was prepared according to the procedure of Example 1A, substituting 1-(2-hydroxyphenyl)ethanone for 1-(5-fluoro-2-hydroxyphenyl)ethanone and using propan-2-one. MS (DCl/NH₃) m/z 194 (M+NH₄)⁺.

### Example 27B

### (R)-2,2-Dimethylchroman-4-amine, (R)-2-hydroxy-2-phenylacetic acid salt

The title compound was prepared from Example 27A according to the methods described in Example 1B, Example 1C, and Example 1D. MS (APCI) m/z 178 (M+H)⁺.

### Example 27C

### N-[(4R)-2,2-Dimethyl-3,4-dihydro-2H-chromen-4-yl]-N′-isoquinolin-5-ylurea

The title compound was prepared according to the procedure of Example 5 substituting Example 27B for Example 1C. ¹H NMR (300 MHz, DMSO-d₆) δ 9.28 (s, 1H), 8.72 (s, 1H), 8.55 (d, J=6.0 Hz, 1H), 8.36 (d, J=8.1 Hz, 1H), 7.94 (d, J=6.1 Hz, 1H), 7.76 (d, J=8.1 Hz, 1H), 7.63 (t, J=7.9 Hz, 1H), 7.34 (d, J=7.8 Hz, 1H), 7.20-7.13 (m, 1H), 7.01-6.88 (m, 2H), 6.76 (dd, J=8.2, 1.2 Hz, 1H), 5.07-4.98 (m, 1H), 2.21 (dd, J=13.2, 6.2 Hz, 1H), 1.86-1.74 (m, 1H), 1.41 (s, 3H), 1.30 (s, 3H). MS (DCl/NH₃) m/z 348 (M+H)⁺; [α]²³_{D}=+ 34.1 (c 0.65, CH₃OH).

### Example 28

### N-[(4R)-2,2-Diethyl-6-fluoro-3,4-dihydro-2H-chromen-4-yl]-N′-isoquinolin-5-ylurea

The title compound was prepared according to the procedure of Example 5 substituting Example 26B for Example 1C. ¹H NMR (300 MHz, DMSO-d₆) δ 9.29 (d, J=0.8 Hz, 1H), 8.73 (s, 1H), 8.56 (d, J=6.0 Hz, 1H), 8.34 (dd, J=7.7, 1.1 Hz, 1H), 7.94 (d, J=6.1 Hz, 1H), 7.77 (d, J=8.1 Hz, 1H), 7.63 (t, J=7.9 Hz, 1H), 7.09-7.14 (m, 1H), 6.98-7.05 (m, 2H), 6.81 (dd, J=8.9, 4.9 Hz, 1H), 4.93-5.02 (m, 1H), 2.20 (dd, J=13.4, 6.1 Hz, 1H), 1.52-1.77 (m, 5H), 0.85-0.94 (m, 6H); MS (DCl/NH₃) m/z 394 (M+H)⁺; [α]²³_{D}=+ 34.1 (c 0.46, CH₃OH).

### Example 29

### N-[(4R)-7-Fluoro-2,2-dimethyl-3,4-dihydro-2H-chromen-4-yl]-N′-1H-indazol-4-ylurea

The title compound was prepared according to the procedure of Example 2D, substituting Example 23B for Example 1D. ¹H NMR (300 MHz, DMSO-d₆) δ 13.02 (br s, 1H), 8.71 (s, 1H), 8.07 (s, 1H), 7.68 (d, J=7.5 Hz, 1H), 7.37-7.32 (m, 1H), 7.23 (t, J=7.9 Hz, 1H), 7.09 (d, J=8.2 Hz, 1H), 6.79-6.71 (m, 2H), 6.61 (dd, J=10.6, 2.6 Hz, 1H), 5.03-4.93 (m, 1H), 2.20 (dd, J=13.3, 6.1 Hz, 1H), 1.78 (dd, J=13.2, 10.8 Hz, 1H), 1.42 (s, 3H), 1.31 (s, 3H); MS (DCl/NH₃) m/z 355 (M+H)⁺; [α]²³_{D}=+ 34.7 (c 1.0, CH₃OH).

### Example 30

### N-[(4R)-7-Fluoro-2,2-dimethyl-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 23B for Example 1D. ¹H NMR (300 MHz, DMSO-d₆) δ 7.70 (d, J=7.9 Hz, 1H), 7.59 (s, 1H), 7.34-7.25 (m, 1H), 7.01 (t, J=7.8 Hz, 1H), 6.94 (d, J=8.4 Hz, 1H), 6.80-6.69 (m, 2H), 6.59 (dd, J=10.6, 2.6 Hz, 1H), 4.98-4.88 (m, 1H), 4.86 (d, J=4.1 Hz, 1H), 4.00-3.88 (m, 1H), 2.90-2.63 (m, 3H), 2.34 (dd, J=16.6, 7.7 Hz, 1H), 2.16 (dd, J=13.3, 6.1 Hz, 1H), 1.94-1.81 (m, 1H), 1.70 (dd, J=13.2, 10.9 Hz, 1H), 1.65-1.51 (m, 1H), 1.40 (s, 3H), 1.29 (s, 3H); MS (ESI) m/z 385 (M+H)⁺; [α]²³_{D}=+ 20.2° (c 1.0, CH₃OH).

### Example 31

### N-[(4R)-7-Fluoro-2,2-dimethyl-3,4-dihydro-2H-chromen-4-yl]-N′-[(7S)-7-hydroxy-5,6,7,8-tetrahydronaphthalen-1-yl]urea

The title compound was prepared according to the procedure of Example 3C, substituting Example 23B for Example 1D. ¹H NMR (300 MHz, DMSO-d₆) δ 7.70 (d, J=7.5 Hz, 1H), 7.58 (s, 1H), 7.35-7.25 (m, 1H), 7.01 (t, J=7.8 Hz, 1H), 6.94 (d, J=8.3 Hz, 1H), 6.79-6.69 (m, 2H), 6.59 (dd, J=10.6, 2.6 Hz, 1H), 4.98-4.88 (m, 1H), 4.86 (d, J=4.1 Hz, 1H), 3.99-3.86 (m, 1H), 2.90-2.63 (m, 3H), 2.35 (dd, J=16.3, 7.5 Hz, 1H), 2.15 (dd, J=13.2, 6.1 Hz, 1H), 1.93-1.82 (m, 1H), 1.70 (dd, J=13.4, 10.9 Hz, 1H), 1.65-1.51 (m, 1H), 1.40 (s, 3H), 1.28 (s, 3H); MS (ESI) m/z 385 (M+H)⁺; [α]²³_{D}=+ 26.0° (c 1.0, CH₃OH).

### Example 32

### N-[(4R)-8-Fluoro-2,2-dimethyl-3,4-dihydro-2H-chromen-4-yl]-N′-1H-indazol-4-ylurea

The title compound was prepared according to the procedure of Example 2D, substituting Example 20B for Example 1D. ¹H NMR (300 MHz, DMSO-d₆) δ 13.02 (br s, 1H), 8.74 (s, 1H), 8.08 (s, 1H), 7.67 (d, J=7.5 Hz, 1H), 7.23 (t, J=7.9 Hz, 1H), 7.14 (d, J=7.4 Hz, 1H), 7.11-7.07 (m, 2H), 6.88 (td, J=8.0, 5.0 Hz, 1H), 6.77 (d, J=8.4 Hz, 1H), 5.09-4.99 (m, 1H), 2.23 (dd, J=13.3, 6.2 Hz, 1H), 1.84 (dd, J=13.3, 10.9 Hz, 1H), 1.46 (s, 3H), 1.33 (s, 3H); MS (DCl/NH₃) m/z 355 (M+H)⁺; [α]²³_{D}=+ 28.7° (c 0.32, CH₃OH).

### Example 33

### N-[(4R)-2,2-Dimethyl-7-(trifluoromethyl)-3,4-dihydro-2H-chromen-4-yl]-N′-(1-methyl-1H-indazol-4-yl)urea

### Example 33A

### 2,2-Dimethyl-7-(trifluoromethyl)chroman-4-one

A solution of 2-hydroxy-4-(trifluoromethyl)benzoic acid (10.0 g, 48.5 mmol) and THF (100 mL) was cooled to <5° C. (internal temperature) and methyllithium (95 mL of a 1.6M solution in Et₂O, 152 mmol) was added, keeping the internal temperature <20° C. (slow addition, methane generation). Following methyllithium addition, the solution was warmed to ambient temperature and stirred for 1 h. The solution was then re-cooled to 10° C. and treated carefully with EtOAc (100 mL) and 2N HCl (100 mL). The reaction mixture was further diluted with EtOAc (100 mL) then washed with water (100 mL) and brine (20 mL). The organic portion was dried (Na₂SO₄), filtered, and concentrated to give 1-(2-hydroxy-4-(trifluoromethyl)phenyl)ethanone (10.3 g) which was used without further purification.

The crude 1-[2-hydroxy-4-(trifluoromethyl)phenyl]ethanone (9.90 g, 48.5 mmol) from above was dissolved in methanol (100 mL) and acetone (3.56 mL, 48.5 mmol), and pyrrolidine (8.02 mL, 97.0 mmol) were added. The reaction was stirred at ambient temperature for 14 h; LCMS showed reaction completion. The reaction mixture was concentrated and diluted with EtOAc (300 mL), then washed with water (100 mL), 2N HCl (2×100 mL), water (50 mL), 2N NaOH (2×100 mL), water (50 mL), and brine (20 mL). The organic portion was dried (Na₂SO₄), filtered, concentrated, and the residue purified by silica gel chromatography (gradient elution, 0-20% EtOAc/hexanes) to give the title compound (8.93 g, 36.6 mmol, 75%) as a white solid. MS (ESI) m/z 245 (M+H)⁺.

### Example 33B

### (R)-7-(Trifluoromethyl)-2,2-dimethylchroman-4-amine, (R)-2-hydroxy-2-phenylacetic acid salt

The title compound was prepared from Example 33A according to the methods described in Example 1B, Example 1C, and Example 1D. MS (DCl/NH₃) m/z 246 (M+H)⁺.

### Example 33C

### N-[(4R)-2,2-Dimethyl-7-(trifluoromethyl)-3,4-dihydro-2H-chromen-4-yl]-N′-(1-methyl-1H-indazol-4-yl)urea

The title compound was prepared according to the procedure of Example 1H, substituting Example 33B for Example 1D. ¹H NMR (300 MHz, DMSO-d₆) δ 8.81 (s, 1H), 8.05 (d, J=0.9 Hz, 1H), 7.70 (d, J=7.5 Hz, 1H), 7.54 (d, J=8.1 Hz, 1H), 7.32-7.23 (m, 2H), 7.18 (d, J=8.3 Hz, 1H), 7.07 (d, J=1.7 Hz, 1H), 6.80 (d, J=8.4 Hz, 1H), 5.12-5.03 (m, 1H), 3.28 (s, 3H), 2.23 (dd, J=13.2, 6.2 Hz, 1H), 1.86 (dd, J=13.2, 11.2 Hz, 1H), 1.42 (s, 3H), 1.32 (s, 3H); MS (DCl/NH₃) m/z 419 (M+H)⁺; [α]²³_{D}=+16°(c 0.78, CH₃OH).

### Example 34

### N-[(4R)-2,2-Dimethyl-7-(trifluoromethyl)-3,4-dihydro-2H-chromen-4-yl]-N′-isoquinolin-5-ylurea

The title compound was prepared according to the procedure of Example 5 substituting Example 33B for Example 1C. ¹H NMR (300 MHz, DMSO-d₆) δ 9.29 (d, J=0.8 Hz, 1H), 8.78 (s, 1H), 8.56 (d, J=6.0 Hz, 1H), 8.33 (dd, J=7.7, 1.1 Hz, 1H), 7.94 (d, J=6.1 Hz, 1H), 7.78 (d, J=8.1 Hz, 1H), 7.64 (t, J=7.9 Hz, 1H), 7.56 (d, J=8.1 Hz, 1H), 7.26 (dd, J=8.1, 1.8 Hz, 1H), 7.08-7.04 (m, 2H), 5.14-5.04 (m, 1H), 2.25 (dd, J=13.3, 6.2 Hz, 1H), 1.87 (dd, J=13.2, 11.1 Hz, 1H), 1.45 (s, 3H), 1.33 (s, 3H); MS (DCl/NH₃) m/z 416 (M+H)⁺; [α]²³_{D}=+ 26.8° (c 0.50, CH₃OH).

### Example 35

### N-[(4R)-6-Fluoro-2,2-dimethyl-3,4-dihydro-2H-chromen-4-yl]-N′-(3-methylisoquinolin-5-yl)urea

### Example 35A

### 3-Methyl-5-nitroisoquinoline

To a 0° C. solution of 3-methylisoquinoline (3.00 g, 20.9 mmol) in concentrated sulfuric acid (35 mL) was added solid potassium nitrate (2.33 g, 23.0 mmol) in four portions. The mixture was stirred 2 h at 0° C. then was diluted with ice. This mixture was basified (pH 10) with 50% aqueous NaOH extracted with CH₂Cl₂ (60 mL). The organic phase was washed with brine (25 mL), dried (Na₂SO₄), filtered, and the volatiles were removed in vacuo. The resulting solid was triturated with 1:1 EtOAc-hexanes, filtered and air-dried to provide the title compound (1.60, 8.78 mmol, 42%) as a yellow solid.

¹H NMR (300 MHz, CDCl₃) δ 9.30 (s, 1H), 8.53 (dd, J=7.7, 1.1 Hz, 1H), 8.35 (s, 1H), 8.26 (d, J=8.1 Hz, 1H), 7.64 (dd, J=9.9, 5.9 Hz, 1H), 2.80 (s, 3H); MS (ESI) m/z 189 (M+H)⁺.

### Example 35B

### 3-Methylisoquinolin-5-amine

To a solution of Example 35A (1.60 g, 8.82 mmol) in ethanol (45 mL) and THF (45 mL) was added 10% Pd/C (100 mg). The solution was hydrogenated under 1 atmosphere of hydrogen for 16 h at ambient temperature. The mixture was filtered through a plug of Celite and the volatiles were evaporated in vacuo. The resulting solid was triturated with 1:1 CH₂Cl₂-hexanes and air-dried to provide the title compound (1.31 g, 8.29 mmol, 94% yield) as a light green solid. ¹H NMR (300 MHz, DMSO-d₆) δ 9.00 (s, 1H), 7.78 (d, J=0.6 Hz, 1H), 7.25 (d, J=7.5 Hz, 1H), 7.18 (d, J=8.0 Hz, 1H), 6.80 (dd, J=7.4, 1.2 Hz, 1H), 5.84 (s, 2H), 2.58 (s, 3H); MS (ESI) m/z 159 (M+H)⁺.

### Example 35C

### N-[(4R)-6-Fluoro-2,2-dimethyl-3,4-dihydro-2H-chromen-4-yl]-N′-(3-methylisoquinolin-5-yl)urea

The title compound was prepared according to the procedure of Example 5 substituting Example 35B for 5-aminoisoquinoline. ¹H NMR (300 MHz, DMSO-d₆) δ 9.19 (s, 1H), 8.65 (s, 1H), 8.27 (d, J=7.6 Hz, 1H), 7.72 (d, J=8.2 Hz, 1H), 7.53 (t, J=7.9 Hz, 1H), 7.12 (dd, J=9.5, 3.0 Hz, 1H), 7.02 (td, J=8.4, 3.3 Hz, 2H), 6.79 (dd, J=8.9, 4.9 Hz, 1H), 5.00 (dd, J=17.8, 7.4 Hz, 1H), 2.59 (d, J=14.2 Hz, 3H), 2.20 (dd, J=13.2, 6.2 Hz, 1H), 1.83-1.71 (m, 1H), 1.38 (d, J=19.2 Hz, 3H), 1.30-1.19 (m, 3H); MS (ESI) m/z 380 (M+H)⁺.

### Example 36

### N-[(4R)-2,2-Dimethyl-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 27B for Example 1D. ¹H NMR (300 MHz, DMSO-d₆) δ 7.71 (d, J=7.8 Hz, 1H), 7.58 (s, 1H), 7.28 (d, J=7.6 Hz, 1H), 7.14 (t, J=7.7 Hz, 1H), 7.01 (t, J=7.8 Hz, 1H), 6.94 (d, J=8.4 Hz, 1H), 6.89 (td, J=7.6, 1.1 Hz, 1H), 6.77-6.70 (m, 2H), 5.01-4.89 (m, 1H), 4.86 (d, J=4.1 Hz, 1H), 3.99-3.87 (m, 1H), 2.90-2.64 (m, 3H), 2.34 (dd, J=16.5, 7.7 Hz, 1H), 2.15 (dd, J=613.2, 6.2 Hz, 1H), 1.93-1.82 (m, 1H), 1.69 (dd, J=13.2, 10.8 Hz, 1H), 1.63-1.51 (m, 1H), 1.39 (s, 3H), 1.28 (s, 3H); MS (ESI) m/z 367 (M+H)⁺; [α]²³_{D}+28.0° (c 1.0, CH₃OH).

### Example 37

### N-[(4R)-2,2-Dimethyl-3,4-dihydro-2H-chromen-4-yl]-N′-[(7S)-7-hydroxy-5,6,7,8-tetrahydronaphthalen-1-yl]urea

The title compound was prepared according to the procedure of Example 3C, substituting Example 27B for Example 1D. ¹H NMR (300 MHz, DMSO-d₆) δ 7.72 (d, J=7.3 Hz, 1H), 7.57 (s, 1H), 7.28 (d, J=7.6 Hz, 1H), 7.14 (td, J=7.5, 1.2 Hz, 1H), 7.01 (t, J=7.8 Hz, 1H), 6.96-6.84 (m, 2H), 6.73 (dd, J=8.2, 1.2 Hz, 2H), 5.01-4.89 (m, 1H), 4.85 (d, J=4.2 Hz, 1H), 3.98-3.87 (m, 1H), 2.89-2.64 (m, 3H), 2.35 (dd, J=16.4, 7.7 Hz, 1H), 2.15 (dd, J=13.2, 6.2 Hz, 1H), 1.92-1.82 (m, 1H), 1.69 (dd, J=13.1, 10.9 Hz, 1H), 1.64-1.52 (m, 1H), 1.39 (s, 3H), 1.27 (s, 3H); MS (ESI) m/z 367 (M+H)⁺; [α]²³D+33.5° (c 1.0, CH₃OH).

### Example 38

### N-[(4R)-6-Fluoro-2,2-dimethyl-3,4-dihydro-2H-chromen-4-yl]-N′-isoquinolin-8-ylurea

The title compound was prepared according to the procedure of Example 5 substituting 8-aminoisoquinoline (Combi-Blocks) for 5-aminoisoquinoline. ¹H NMR (300 MHz, DMSO-d₆) δ 9.52 (s, 1H), 9.00 (s, 1H), 8.51 (d, J=5.7 Hz, 1H), 8.18 (dd, J=7.6, 0.8 Hz, 1H), 7.80 (d, J=5.2 Hz, 1H), 7.71 (t, J=7.9 Hz, 1H), 7.60 (d, J=8.1 Hz, 1H), 7.13 (dd, J=9.5, 2.5 Hz, 1H), 7.01 (dd, J=13.4, 3.6 Hz, 1H), 6.79 (dd, J=8.9, 4.9 Hz, 1H), 5.01 (dd, J=17.9, 7.3 Hz, 1H), 2.21 (dd, J=13.2, 6.2 Hz, 1H), 1.79 (dd, J=13.1, 11.0 Hz, 1H), 1.41 (s, 3H), 1.29 (s, 3H); MS (ESI) m/e 366 (M+H)⁺.

### Example 39

### N-[(4R)-2,2-Dimethyl-7-(trifluoromethoxy)-3,4-dihydro-2H-chromen-4-yl]-N′-(1-methyl-1H-indazol-4-yl)urea

### Example 39A

### 2,2-Dimethyl-7-(trifluoromethoxy)chroman-4-one

Eaton's reagent (225 mL) was heated to 70 2C and 3-methylbut-2-enoic acid (28.1 g, 281 mmol) and 3-(trifluoromethoxy)phenol (25.0 g, 140 mmol) were added. After 30 min, additional 3-methylbut-2-enoic acid (1 equiv, 14 g) was added and heating was continued. After 30 min, additional Eaton's reagent (150 mL) was added and heating was continued for 35 min. The dark solution was cooled and poured into ice. The aqueous suspension was extracted with Et₂O (300 mL), and the organic portion was washed with water (75 mL) and brine (50 mL). The organic portion was dried (Na₂SO₄), filtered, concentrated, and purified by silica gel chromatography (gradient elution, 0-20% EtOAc/hexanes) to give the title compound (11.7 g, 45.0 mmol, 32%) as a white solid. MS (ESI) m/z 261 (M+H)⁺.

### Example 39B

### (R)-7-(Trifluoromethoxy)-2,2-dimethylchroman-4-amine, (R)-2-hydroxy-2-phenylacetic acid salt

The title compound was prepared from Example 39A according to the methods described in Example 1B, Example 1C, and Example 1D. MS (DCl/NH₃⁺) m/z 262 (M+H)⁺.

### Example 39C

### N-[(4R)-2,2-Dimethyl-7-(trifluoromethoxy)-3,4-dihydro-2H-chromen-4-yl]-N′-(1-methyl-1H-indazol-4-yl)urea

The title compound was prepared according to the procedure of Example 1H, substituting Example 39B for Example 1D. ¹H NMR (300 MHz, DMSO-d₆) δ 8.75 (s, 1H), 8.04 (d, J=0.9 Hz, 1H), 7.70 (dd, J=7.5, 0.8 Hz, 1H), 7.43 (dd, J=8.5, 1.0 Hz, 1H), 7.27 (d, J=7.7 Hz, 1H), 7.17 (dt, J=8.4, 0.8 Hz, 1H), 6.91 (ddd, J=8.5, 2.5, 1.2 Hz, 1H), 6.78-6.73 (m, 2H), 5.06-4.97 (m, 1H), 4.01 (s, 3H) 2.28-2.18 (m, 1H), 1.82 (dd, J=13.3, 10.9 Hz, 1H), 1.43 (s, 3H), 1.32 (s, 3H); MS (DCl/NH₃) m/z 435 (M+H)⁺; [α]²³D+6.2° (c 0.53, CH₃OH).

### Example 40

### N-[(4R)-2,2-Dimethyl-7-(trifluoromethoxy)-3,4-dihydro-2H-chromen-4-yl]-N′-isoquinolin-5-ylurea

The title compound was prepared according to the procedure of Example 5 substituting Example 39B for Example 1C. ¹H NMR (300 MHz, DMSO-d₆) δ 9.29 (d, J=0.8 Hz, 1H), 8.78 (s, 1H), 8.56 (d, J=6.0 Hz, 1H), 8.34 (dd, J=7.7, 1.1 Hz, 1H), 7.93 (d, J 6.1 Hz, 1H), 7.77 (d, J=8.1 Hz, 1H), 7.63 (t, J=7.9 Hz, 1H), 7.45 (dd, J=8.5, 1.0 Hz, 1H), 7.02 (d, J=8.3 Hz, 1H), 6.92 (ddd, J=8.5, 2.5, 1.3 Hz, 1H), 6.75 (dd, J=2.5, 1.1 Hz, 1H), 5.08-4.99 (m, 1H), 2.22 (dd, J=13.3, 6.1 Hz, 1H), 1.83 (dd, J=13.3, 10.8 Hz, 1H), 1.45 (s, 3H), 1.33 (s, 3H); MS (DCl/NH₃) m/z 432 (M+H)⁺; [α]²³_{D}+7.5° (c 0.45, CH₃OH).

### Example 41

### N-[(4R)-2,2-Dimethyl-7-(trifluoromethyl)-3,4-dihydro-2H-chromen-4-yl]-N′-1H-indazol-4-ylurea

The title compound was prepared according to the procedure of Example 2D, substituting Example 33B for Example 1D. ¹H NMR (300 MHz, DMSO-d₆) δ 13.02 (br s, 1H), 8.77 (s, 1H), 8.09 (s, 1H), 7.67 (d, J=7.2 Hz, 1H), 7.54 (d, J=8.1 Hz, 1H), 7.24 (t, J=8.0 Hz, 2H), 7.10 (d, J=8.2 Hz, 1H), 7.07 (d, J=1.8 Hz, 1H), 6.81 (d, J=8.4 Hz, 1H), 5.12-5.03 (m, 1H), 2.23 (dd, J=13.2, 6.1 Hz, 1H), 1.90-1.81 (m, 1H), 1.44 (s, 3H), 1.33 (s, 3H); MS (DCl/NH₃) m/z 405 (M+H)⁺; [α]²³D+21.4° (c 0.30, CH₃OH).

### Example 42

### N-[(4R)-2,2-Dimethyl-8-(trifluoromethyl)-3,4-dihydro-2H-chromen-4-yl]-N′-(1-methyl-1H-indazol-4-yl)urea

### Example 42A

### 1-(Methoxymethoxy)-2-(trifluoromethyl)benzene

A solution of 2-(trifluoromethyl)phenol (12.0 g, 74.0 mmol) in dichloromethane (49 mL) was cooled to 5° C., and N,N-diisopropylethylamine (25.9 mL, 148 mmol) and methoxymethyl chloride (8.43 mL, 111 mmol) were added dropwise, keeping the internal temperature <15° C. After stirring for 15 min at ambient temperature, the reaction mixture was diluted with MTBE (250 mL) and washed with 2N HCl (2×50 mL), water (50 mL), 2N NaOH (2×30 mL), water (30 mL), and brine (30 mL). The organic portion was dried (Na₂SO₄), filtered, and concentrated to give the title compound (14.1 g, 68.4 mmol, 92%) which was used without further purification. MS (DCl/NH₃) m/z 207 (M+H)⁺.

### Example 42B

### 2-Hydroxy-3-(trifluoromethyl)benzoic acid

A solution of Example 42A (14.1 g, 68.4 mmol) in THF (68 mL) was cooled to −20° C. and n-butyllithium (30.1 mL of a 2.5 M solution in hexanes, 75.0 mmol) was added slowly, keeping the temperature at 0° C. After 70 min at −5 to 5° C., the reaction mixture was cooled to −20° C. and CO₂ gas was bubbled through the brown slurry, keeping the temperature ≦−10° C. The reaction went from a brown slurry to a dark purple solution to a yellow solution. After 10 min, the reaction mixture was cooled further to −20° C. and treated with 2N HCl (68 mL, 140 mmol). To facilitate the reaction mixture, additional concentrated HCl (17 mL, total 5 equiv of 4M HCl) was added. After 30 min, MTBE (70 mL) was added, and the organic portion was extracted with 2N NaOH (70 mL) and water (70 mL). The aqueous layer was acidified with 2N HCl (98 mL) and extracted with dichloromethane (2×140 mL). The organic portion was dried (Na₂SO₄), filtered, and concentrated to give the title compound (14.8 g, 71.8 mmol, 99%) as a yellow solid which was used without further purification. MS (DCl/NH₃) m/z 207 (M+H)⁺.

### Example 42C

### 1-(2-Hydroxy-3-(trifluoromethyl)phenyl)ethanone

A solution of Example 42B (14.1 g, 68.4 mmol) in THF (70 mL) was cooled to 5° C. and methyllithium (133 mL of a 1.6M solution in Et₂O, 212 mmol) was added, keeping the temperature ≦20° C. (slow addition, methane generation). The cooling bath was removed and after 10 min, the reaction mixture was complete by LCMS. The reaction was cooled to 10° C. and EtOAc (140 mL) and 2N HCl (140 mL) were added. The layers were partitioned and the organic portion was washed with water (70 mL) and brine (28 mL). The organic portion was dried (Na₂SO₄), filtered, and concentrated, to give the title compound (14.0 g, 68.6 mmol, 99%) as an orange oil that was used without further purification. MS (DCl/NH₃) m/z 222 (M+NH₄)⁺.

### Example 42D

### 2,2-Dimethyl-8-(trifluoromethyl)chroman-4-one

A solution of crude Example 42C (13.9 g, 68.4 mmol), methanol (140 mL), 2-propanone (10.1 mL, 137 mmol), and pyrrolidine (6.22 ml, 75.0 mmol) were stirred at ambient temperature for 16 h. EtOAc (430 mL) was added and the solution was washed with water (140 mL), 2N HCl (2×70 mL), water (70 mL), 2N NaOH (2×70 mL), water (70 mL), and brine (30 mL). The organic portion was dried (Na₂SO₄), filtered, and concentrated. The resulting residue was purified by silica gel chromatography (gradient elution, 0-25% EtOAc/hexanes) to give the title compound (9.04 g, 37.0 mmol, 54% overall yield) as an off-white solid. MS (DCl/NH₃) m/z 262 (M+NH₄)⁺.

### Example 42E

### (R)-8-(Trifluoromethyl)-2,2-dimethylchroman-4-amine, (R)-2-hydroxy-2-phenylacetic acid salt

The title compound was prepared from Example 42D according to the methods described in Example 1B, Example 1C, and Example 1D. MS (DCl/NH₃⁺) m/z 246 (M+H)⁺.

### Example 42F

### N-[(4R)-2,2-Dimethyl-8-(trifluoromethyl)-3,4-dihydro-2H-chromen-4-yl]-N′-(1-methyl-1H-indazol-4-yl)urea

The title compound was prepared according to the procedure of Example 1H, substituting Example 42E for Example 1D. ¹H NMR (300 MHz, DMSO-d₆) δ ppm 8.79 (s, 1H), 8.05 (d, J=0.9 Hz, 1H), 7.69 (dd, J=7.5, 0.7 Hz, 1H), 7.59-7.63 (m, 1H), 7.51 (d, J=7.8 Hz, 1H), 7.27 (d, J=7.7 Hz, 1H), 7.19-7.16 (m, 1H), 7.06 (t, J=7.7 Hz, 1H), 6.79 (d, J=8.4 Hz, 1H), 5.11-5.01 (m, 1H), 4.01 (s, 3H), 2.25 (dd, J=13.3, 6.3 Hz, 1H), 1.90 (dd, J=13.3, 10.8 Hz, 1H), 1.44 (s, 3H), 1.33 (s, 3H); MS (DCl/NH₃) m/Z₄₁₉ (M+H)⁺; [α]²³_{D}+14° (c 0.68, CH₃OH).

### Example 43

### N-[(4R)-2,2-Dimethyl-7-(trifluoromethyl)-3,4-dihydro-2H-chromen-4-yl]-N′-isoquinolin-8-ylurea

The title compound was prepared according to the procedure of Example 5 substituting Example 33B for Example 1C, and substituting 8-aminoisoquinoline for isoquinolin-5-amine. ¹H NMR (300 MHz, DMSO-d₆) δ 9.52 (s, 1H), 9.04 (s, 1H), 8.51 (d, J=5.7 Hz, 1H), 8.21-8.15 (m, 1H), 7.80 (dd, J=5.7, 0.5 Hz, 1H), 7.71 (t, J=7.9 Hz, 1H), 7.64-7.55 (m, 2H), 7.29-7.23 (m, 1H), 7.06 (d, J=8.5 Hz, 2H), 5.07 (d, J=8.3 Hz, 1H), 2.25 (dd, J=13.3, 6.2 Hz, 1H), 1.94-1.82 (m, 1H), 1.45 (s, 3H), 1.33 (s, 3H); MS (DCl/NH₃) m/z 416 (M+H)⁺.

### Example 44

### N-[(4R)-2,2-Dimethyl-8-(trifluoromethyl)-3,4-dihydro-2H-chromen-4-yl]-N′-isoquinolin-5-ylurea

The title compound was prepared according to the procedure of Example 5 substituting Example 42E for Example 1C. ¹H NMR (300 MHz, DMSO-d₆) δ 9.31 (s, 1H), 8.75 (s, 1H), 8.56 (d, J=6.0 Hz, 1H), 8.33 (d, J=7.7 Hz, 1H), 7.94 (d, J=6.1 Hz, 1H), 7.78 (d, J=8.1 Hz, 1H), 7.60-7.66 (m, 2H), 7.52 (d, J=7.8 Hz, 1H), 7.06 (t, J=8.3 Hz, 2H), 5.13-5.03 (m, 1H), 2.27 (dd, J=13.3, 6.3 Hz, 1H), 2.00-1.86 (m, 1H), 1.44 (s, 3H), 1.33 (s, 3H); MS (DCl/NH₃) m/z 416 (M+H)⁺; [α]²³_{D}+23.8° (c 0.65, CH₃OH).

### Example 45

### N-[(4R)-2,2-Dimethyl-8-(trifluoromethyl)-3,4-dihydro-2H-chromen-4-yl]-N′-1H-indazol-4-ylurea

The title compound was prepared according to the procedure of Example 2D, substituting Example 42E for Example 1D. ¹H NMR (300 MHz, DMSO-d₆) δ 13.02 (br s, 1H), 8.77 (s, 1H), 8.09 (s, 1H), 7.66 (d, J=7.5 Hz, 1H), 7.63-7.59 (m, 1H), 7.51 (d, J=7.8 Hz, 1H), 7.23 (t, J=7.9 Hz, 1H), 7.16-6.94 (m, 2H), 6.81 (d, J=8.4 Hz, 1H), 5.11-5.02 (m, 1H), 2.26 (dd, J=13.3, 6.2 Hz, 1H), 1.90 (dd, J=13.3, 10.9 Hz, 1H), 1.44 (s, 3H), 1.33 (s, 3H); MS (DCl/NH₃) m/z 405 (M+H)⁺; [α]²³_{D}+13.8° (c 0.45, CH₃OH).

### Example 46

### N-[(4R)-2,2-Diethyl-7-fluoro-3,4-dihydro-2H-chromen-4-yl]-N′-(1-methyl-1H-indazol-4-yl)urea

### Example 46A

### 2,2-Diethyl-7-fluorochroman-4-one

The title compound was prepared according to the procedure of Example 26A, substituting 1-(4-fluoro-2-hydroxyphenyl)ethanone for 1-(5-fluoro-2-hydroxyphenyl)ethanone. MS (ESI) m/z 240 (M+NH₄)⁺.

### Example 46B

### (R)-2,2-Diethyl-7-fluorochroman-4-amine

The title compound was prepared from Example 46A according to the methods described in Example 1B and Example 1C. MS (DCl/NH₃) m/z 224 (M+H)⁺.

### Example 46C

### N-[(4R)-2,2-Diethyl-7-fluoro-3,4-dihydro-2H-chromen-4-yl]-N′-(1-methyl-1H-indazol-4-yl)urea

The title compound was prepared according to the procedure of Example 1H, substituting Example 46B for Example 1D. ¹H NMR (300 MHz, DMSO-d₆) δ 8.73 (s, 1H), 8.04 (d, J=0.9 Hz, 1H), 7.70 (dd, J=7.5, 0.7 Hz, 1H), 7.37-7.25 (m, 2H), 7.17 (d, J=8.3 Hz, 1H), 6.78-6.70 (m, 2H), 6.63 (dd, J=10.6, 2.6 Hz, 1H), 5.00-4.90 (m, 1H), 4.01 (s, 3H), 2.23-2.14 (m, 1H), 1.77-1.51 (m, 5H), 0.99-0.86 (m, 6H); MS (DCl/NH₃) m/z 397 (M+H)⁺; [α]²³_{D}+1.0 (c 0.58, CH₃OH).

### Example 47

### N-[(4R)-2,2-Dimethyl-8-(trifluoromethyl)-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 42E for Example 1D. ¹H NMR (300 MHz, DMSO-d₆) δ 7.69 (d, J=7.8 Hz, 1H), 7.63 (s, 1H), 7.56 (d, J=7.7 Hz, 1H), 7.50 (d, J=7.6 Hz, 1H), 7.10-6.97 (m, 3H), 6.74 (d, J=7.4 Hz, 1H), 5.07-4.94 (m, 1H), 4.86 (d, J=4.2 Hz, 1H), 4.00-3.87 (m, 1H), 2.91-2.64 (m, 3H), 2.35 (dd, J=16.5, 7.7 Hz, 1H), 2.22 (dd, J=13.3, 6.3 Hz, 1H), 1.93-1.75 (m, 2H), 1.67-1.50 (m, 1H), 1.43 (s, 3H), 1.31 (s, 3H); MS (ESI) m/z 435 (M+H)⁺; [α]²³_{D}+28.2° (c 1.0, CH₃OH).

### Example 48

### N-[(4R)-2,2-Diethyl-7-fluoro-3,4-dihydro-2H-chromen-4-yl]-N′-isoquinolin-5-ylurea

The title compound was prepared according to the procedure of Example 5 substituting Example 46B for Example 1C. ¹H NMR (300 MHz, DMSO-d₆) δ 9.28 (d, J=0.8 Hz, 1H), 8.71 (s, 1H), 8.56 (d, J=6.0 Hz, 1H), 8.35 (dd, J=7.7, 1.1 Hz, 1H), 7.93 (d, J=6.1 Hz, 1H), 7.76 (d, J=8.1 Hz, 1H), 7.66-7.57 (m, 1H), 7.39-7.33 (m, 1H), 6.98 (d, J=8.2 Hz, 1H), 6.76 (td, J=8.5, 2.6 Hz, 1H), 6.66-6.56 (m, 1H), 5.01-4.92 (m, 1H), 2.20 (dd, J=13.5, 6.0 Hz, 1H), 1.79-1.54 (m, 5H), 0.95-0.84 (m, 6H); MS (DCl/NH₃) m/z 394 (M+H)⁺; [α]²³_{D}+8.8° (c 0.25, CH₃OH).

### Example 49

### N-[(4R)-22-Diethyl-7-(trifluoromethyl)-3,4-dihydro-2H-chromen-4-yl]-N′-(1-methyl-1H-indazol-4-yl)urea

### Example 49A

### 2,2-Diethyl-7-(trifluoromethyl)chroman-4-one

The title compound was prepared according to the procedure of Example 26A, substituting 1-[2-hydroxy-4-(trifluoromethyl)phenyl]ethanone (prepared as described in Example 33A) for 1-(5-fluoro-2-hydroxyphenyl)ethanone. MS (ESI) m/z 273 (M+H)⁺.

### Example 49B

### (R)-2,2-Diethyl-7-(trifluoromethyl)chroman-4-amine

The title compound was prepared from Example 49A according to the methods described in Example 1B and Example 1C. MS (DCl/NH₃) m/z 274 (M+H)⁺.

### Example 49C

### N-[(4R)-2,2-diethyl-7-(trifluoromethyl)-3,4-dihydro-2H-chromen-4-yl]-N′-(1-methyl-1H-indazol-4-yl)urea

The title compound was prepared according to the procedure of Example 1H, substituting Example 49B for Example 1D. ¹H NMR (300 MHz, DMSO-d₆) δ 8.80 (s, 1H), 8.05 (d, J=0.9 Hz, 1H), 7.70 (d, J=7.5 Hz, 1H), 7.54 (d, J=8.1 Hz, 1H), 7.29 (d, J=8.1 Hz, 1H), 7.27-7.22 (m, 1H), 7.18 (d, J=8.3 Hz, 1H), 7.08 (d, J=1.8 Hz, 1H), 6.80 (d, J=8.4 Hz, 1H), 5.09-4.99 (m, 1H), 4.01 (s, 3H), 2.28-2.19 (m, 1H), 1.85-1.53 (m, 5H), 0.96-0.87 (m, 6H); MS (DCl/NH₃) m/z 447 (M+H)⁺; [α]²³_{D}+8.6° (c 0.57, CH₃OH).

### Example 50

### N-[(4R)-2,2-Diethyl-8-fluoro-3,4-dihydro-2H-chromen-4-yl]-N′-(1-methyl-1H-indazol-4-yl)urea

### Example 50A

### 2,2-Diethyl-8-fluorochroman-4-one

The title compound was prepared according to the procedure of Example 26A, substituting 1-(3-fluoro-2-hydroxyphenyl)ethanone for 1-(5-fluoro-2-hydroxyphenyl)ethanone. MS (DCl/NH₃) m/z 240 (M+NH₄)⁺.

### Example 50B

### (R)-2,2-Diethyl-8-fluorochroman-4-amine

The title compound was prepared from Example 50A according to the methods described in Example 1B and Example 1C. MS (DCl/NH₃⁺) m/z 224 (M+H)⁺.

### Example 50C

### N-[(4R)-2,2-Diethyl-8-fluoro-3,4-dihydro-2H-chromen-4-yl]-N′-(1-methyl-1H-indazol-4-yl)urea

The title compound was prepared according to the procedure of Example 1H, substituting Example 50B for Example 1D. ¹H NMR (300 MHz, DMSO-d₆) δ 8.75 (s, 1H), 8.04 (d, J=0.9 Hz, 1H), 7.70 (dd, J=7.5, 0.8 Hz, 1H), 7.27 (d, J=7.7 Hz, 1H), 7.06-7.19 (m, 3H), 6.88 (td, J=7.9, 5.0 Hz, 1H), 6.76 (d, J=8.3 Hz, 1H), 4.01 (s, 3H), 2.28-2.19 (m, 1H), 1.83-1.58 (m, 6H), 0.96-0.87 (m, 6H); MS (DCl/NH₃) m/z 397 (M+H)⁺; [α]²³_{D}+7.2° (c 0.57, CH₃OH).

### Example 51

### N-[(4R)-2,2-Diethyl-7-(trifluoromethyl)-3,4-dihydro-2H-chromen-4-yl]-N′-isoquinolin-5-ylurea

The title compound was prepared according to the procedure of Example 5 substituting Example 49B for Example 1C. ¹H NMR (300 MHz, DMSO-d₆) δ 9.29 (d, J=0.6 Hz, 1H), 8.76 (s, 1H), 8.56 (d, J=6.0 Hz, 1H), 8.33 (dd, J=7.6, 1.0 Hz, 1H), 7.94 (d, J=6.1 Hz, 1H), 7.78 (d, J=8.1 Hz, 1H), 7.68-7.49 (m, 2H), 7.25 (d, J=8.1 Hz, 1H), 7.07 (dd, J=11.8, 4.9 Hz, 2H), 5.04 (s, 1H), 2.24 (dd, J=13.6, 6.2 Hz, 1H), 1.80-1.50 (m, 5H), 1.00-0.80 (m, 6H); MS (DCl/NH₃) m/z 444 (M+H)⁺; [α]²³_{D}+24.3° (c 0.14, CH₃OH).

### Example 52

### N-[(4R)-2,2-Diethyl-8-fluoro-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 50B for Example 1D. ¹H NMR (300 MHz, DMSO-d₆) δ 7.70 (d, J=7.5 Hz, 1H), 7.59 (s, 1H), 7.13-6.95 (m, 4H), 6.86 (dt, J=8.0, 5.0 Hz, 1H), 6.73 (d, J=7.4 Hz, 1H), 5.01-4.88 (m, 1H), 4.86 (d, J=4.2 Hz, 1H), 3.97-3.87 (m, 1H), 2.90-2.65 (m, 3H), 2.34 (dd, J=16.5, 7.6 Hz, 1H), 2.18 (dd, J=13.5, 6.1 Hz, 1H), 1.94-1.81 (m, 1H), 1.78-1.50 (m, 6H), 0.90 (dt, J=12.1, 7.4 Hz, 6H); MS (ESI) m/z 413 (M+H)⁺; [α]²³_{D}+22.12 (c 1.0, CH₃OH).

### Example 53

### N-[(4R)-2,2-Dimethyl-7-(trifluoromethyl)-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 33B for Example 1D. ¹H NMR (300 MHz, DMSO-d₆) δ 7.69 (d, J=7.9 Hz, 1H), 7.63 (s, 1H), 7.50 (d, J=8.1 Hz, 1H), 7.24 (dd, J=8.0, 1.2 Hz, 1H), 7.07-6.97 (m, 3H), 6.74 (d, J=7.5 Hz, 1H), 5.08-4.95 (m, 1H), 4.87 (d, J=4.1 Hz, 1H), 4.00-3.86 (m, 1H), 2.91-2.64 (m, 3H), 2.35 (dd, J=16.5, 7.7 Hz, 1H), 2.20 (dd, J=13.3, 6.2 Hz, 1H), 1.93-1.83 (m, 1H), 1.77 (dd, J=13.0, 11.5 Hz, 1H), 1.67-1.51 (m, 1H), 1.43 (s, 3H), 1.31 (s, 3H); MS (ESI) m/z 435 (M+H)⁺; [α]²³_{D}+34.8° (c 1.0, CH₃OH).

### Example 54

### N-[(4R)-2,2-Diethyl-6-fluoro-3,4-dihydro-2H-chromen-4-yl]-N′-[(7R)-7-hydroxy-5,6,7,8-tetrahydronaphthalen-1-yl]urea

The title compound was prepared according to the procedure of Example 3C, substituting Example 4A for Example 3B, and substituting Example 26B for Example 1D.

¹H NMR (300 MHz, DMSO-d₆) δ 7.70 (d, J=7.8 Hz, 1H), 7.60 (s, 1H), 7.08-6.94 (m, 4H), 6.83-6.70 (m, 2H), 4.96-4.84 (m, 2H), 3.98-3.87 (m, 1H), 2.90-2.64 (m, 3H), 2.34 (dd, J=16.4, 7.7 Hz, 1H), 2.15 (dd, J=13.5, 6.2 Hz, 1H), 1.93-1.82 (m, 1H), 1.72-1.47 (m, 6H), 0.88 (dt, J=11.9, 7.4 Hz, 6H); MS (ESI) m/z 413 (M+H)⁺;[□]²³_{D}+26.4° (c 1.0, CH₃OH); MS (DCl/NH₃) m/z 394(M+H)⁺; [α]²³_{D}+8.8° (c 0.25, CH₃OH).

### Example 55

### N-[(4R)-2,2-Diethyl-8-(trifluoromethoxy)-3,4-dihydro-2H-chromen-4-yl]-N′-(1-methyl-1H-indazol-4-yl)urea

### Example 55A

### 1-(Methoxymethoxy)-2-(trifluoromethoxy)benzene

The title compound was prepared according to the procedure of Example 42A, substituting 2-(trifluoromethoxy)phenol for 2-(trifluoromethyl)phenol. MS (DCl/NH₃) m/z 222 (M+H)⁺.

### Example 55B

### 2-Hydroxy-3-(trifluoromethoxy)benzoic acid

The title compound was prepared according to the procedure of Example 42B, substituting Example 55A for Example 42A. MS (DCl/NH₃) m/z 223 (M+H)⁺.

### Example 55C

### 1-(2-Hydroxy-3-(trifluoromethoxy)phenyl)ethanone

The title compound was prepared according to the procedure of Example 42C, substituting Example 55B for Example 42B. MS (DCl/NH₃) m/z 238 (M+NH₄)⁺.

### Example 55D

### 2,2-Diethyl-8-(trifluoromethoxy)chroman-4-one

The title compound was prepared according to the procedure of Example 42D, substituting Example 55C for Example 42C, and substituting 3-pentanone for 2-propanone. MS (DCl/NH₃) m/z 306 (M+NH₄)⁺.

### Example 55E

### (R)-8-(Trifluoromethyl)-2,2-dimethylchroman-4-amine, (R)-2-hydroxy-2-phenylacetic acid salt

The title compound was prepared from Example 55D according to the methods described in Example 1B and Example 1C. MS (DCl/NH₃) m/z 290 (M+H)⁺.

### Example 55F

### N-[(4R)-2,2-Diethyl-8-(trifluoromethoxy)-3,4-dihydro-2H-chromen-4-yl]-N′-(1-methyl-1H-indazol-4-yl)urea

The title compound was prepared according to the procedure of Example 1H, substituting Example 55E for Example 1D. ¹H NMR (300 MHz, DMSO-d₆) δ 8.75 (s, 1H), 8.04 (d, J=0.9 Hz, 1H), 7.70 (d, J=7.5 Hz, 1H), 7.36 (d, J=7.9 Hz, 1H), 7.29 (d, J=8.1 Hz, 1H), 7.25 (d, J=6.7 Hz, 1H), 7.18 (d, J=8.3 Hz, 1H), 6.97 (t, J=7.9 Hz, 1H), 6.80 (d, J=8.3 Hz, 1H), 5.07-4.98 (m, 1H), 4.01 (s, 3H), 2.23 (dd, J=13.6, 6.0 Hz, 1H), 1.84-1.56 (m, 5H), 0.96-0.87 (m, 6H); MS (DCl/NH₃) m/z 463 (M+H)⁺.

### Example 56

### N-[(4R)-2,2-Diethyl-6-fluoro-3,4-dihydro-2H-chromen-4-yl]-N′-(3-methylisoquinolin-5-yl)urea

The title compound was prepared according to the procedure of Example 5 substituting Example 35B for isoquinolin-5-amine, and substituting Example 26B for Example 1 C. ¹H NMR (300 MHz, DMSO-d₆) δ 9.19 (s, 1H), 8.64 (s, 1H), 8.27 (dd, J=7.7, 1.1 Hz, 1H), 7.72 (d, J=8.1 Hz, 1H), 7.53 (t, J=7.9 Hz 1H), 7.12 (dd, J=9.4, 3.2 Hz, 1H), 7.07-6.96 (m, 2H), 6.81 (dd, J=8.9, 4.9 Hz, 1H), 5.04-4.91 (m, 1H), 2.66 (s, 3H), 2.30-2.15 (m, 1H), 1.78-1.50 (m, 5H), 0.96-0.77 (m, 6H); MS (DCl/NH₃) m/z 407 (M+H)⁺.

### Example 57

### N-[(4R)-2,2-Diethyl-8-fluoro-3,4-dihydro-2H-chromen-4-yl]-N′-isoquinolin-5-ylurea

The title compound was prepared according to the procedure of Example 5 substituting Example 50B for Example 1C. ¹H NMR (300 MHz, DMSO-d₆) δ 9.28 (s, 1H), 8.73 (s, 1H), 8.55 (d, J=6.0 Hz, 1H), 8.34 (dd, J=7.7, 1.1 Hz, 1H), 7.93 (d, J=6.1 Hz, 1H), 7.77 (d, J=8.1 Hz, 1H), 7.63 (t, J=7.9 Hz, 1H), 7.18-7.07 (m, 2H), 7.02 (d, J=8.3 Hz, 1H), 6.89 (td, J=7.9, 5.0 Hz, 1H), 5.07-4.98 (m, 1H), 2.24 (dd, J=13.6, 6.1 Hz, 1H), 1.84-1.53 (m, 5H), 0.96-0.87 (m, 6H); MS (DCl/NH₃) m/z 394 (M+H)⁺; [α]²³_{D}+27.9° (c 0.51, CH₃OH).

### Example 58

### N-[(4R)-2,2-Diethyl-6-fluoro-3,4-dihydro-2H-chromen-4-yl]-N′-1H-indazol-4-ylurea

The title compound was prepared according to the procedure of Example 2D, using Example 2C and substituting Example 26B for Example 1D. ¹H NMR (300 MHz, DMSO-d₆) δ 13.01 (br s, 1H), 8.72 (s, 1H), 8.08 (s, 1H), 7.67 (d, J=7.2 Hz, 1H), 7.22 (d, J=7.8 Hz, 1H), 6.97-7.11 (m, 3H), 6.83-6.76 (m, 2H), 5.01-4.91 (m, 1H), 2.19 (dd, J=13.4, 6.2 Hz, 1H), 1.76-1.52 (m, 5H), 0.94-0.85 (m, 6H); MS (DCl/NH₃) m/z 383 (M+H)⁺; [α]²³_{D}+31.6° (c 0.76, CH₃OH).

### Example 59

### N-(1-Methyl-1H-indazol-4-yl)-N′-[(4R)-8-(trifluoromethyl)-3,4-dihydro-2H-chromen-4-yl]urea

### Example 59A

### 8-(Trifluoromethyl)chroman-4-one

The title compound was prepared according to the procedure of Example 42D, substituting paraformaldehyde for 2-propanone. MS (DCl/NH₃) m/z 234 (M+NH₄)⁺.

### Example 59B

### (R)-8-(Trifluoromethyl)chroman-4-amine, (R)-2-hydroxy-2-phenylacetic acid salt

The title compound was prepared from Example 59A according to the methods described in Example 1B, Example 1C, and Example 1D. MS (DCl/NH₃) m/z 218 (M+H)⁺.

### Example 59C

### N-(1-Methyl-1H-indazol-4-yl)-N′-[(4R)-8-(trifluoromethyl)-3,4-dihydro-2H-chromen-4-yl]urea

The title compound was prepared according to the procedure of Example 1H, substituting Example 59B for Example 1D. ¹H NMR (300 MHz, DMSO-d₆) δ 8.61 (s, 1H), 8.00 (s, 1H), 7.70 (d, J=7.5 Hz, 1H), 7.62 (d, J=7.5 Hz, 1H), 7.55 (d, J=7.8 Hz, 1H), 7.33-7.23 (m, 1H), 7.16 (d, J=8.4 Hz, 1H), 7.08 (app t, J=7.7 Hz, 1H), 6.94 (d, J=7.7 Hz, 1H), 5.08-4.91 (m, 1H), 4.55-4.39 (m, 1H), 4.37-4.23 (m, 1H), 2.31-2.01 (m, 2H); MS (DCl/NH₃) m/z 391 (M+H)⁺;. [α]²³_{D}+82.2° (c 0.55, MeOH).

### Example 60

### N-[(4R)-2,2-Diethyl-6,8-difluoro-3,4-dihydro-2H-chromen-4-yl]-N′-(1-methyl-1H-indazol-4-yl)urea

### Example 60A

### 2,2-Diethyl-6,8-difluorochroman-4-one

The title compound was prepared according to the procedure of Example 26A, substituting 1-(3,5-difluoro-2-hydroxyphenyl)ethanone for 1-(5-fluoro-2-hydroxyphenyl)ethanone. MS (DCl/NH₃) m/z 258 (M+NH₄)⁺.

### Example 60B

### (R)-2,2-Diethyl-6,8-difluorochroman-4-amine

The title compound was prepared from Example 60A according to the methods described in Example 1B and Example 1C. MS (DCl/NH₃) m/z 242 (M+H)⁺.

### Example 60C

### N-[(4R)-2,2-Diethyl-6,8-difluoro-3,4-dihydro-2H-chromen-4-yl]-N′-(1-methyl-1H-indazol-4-yl)urea

The title compound was prepared according to the procedure of Example 1H, substituting Example 60B for Example 1D. ¹H NMR (300 MHz, DMSO-d₆) δ 8.79 (s, 1H); 8.05 (d, J=0.9 Hz, 1H), 7.68 (d, J=7.0 Hz, 1H), 7.37-7.09 (m, 3H), 6.96 (d, J=9.3 Hz, 1H), 6.80 (d, J=8.3 Hz, 1H), 4.98 (t, J=12.6 Hz, 1H), 4.02 (d, J=10.5 Hz, 3H), 2.23 (dd, J=13.6, 6.2 Hz, 1H), 1.90-1.49 (m, 5H), 0.90 (dt, J=10.9, 7.5 Hz, 6H);

MS (DCl/NH₃) m/z 415 (M+H)⁺; [α]²³D+14° (c 0.58, CH₃OH).

### Example 61

### N-[(4R)-6-fluoro-2,2-dipropyl-3,4-dihydro-2H-chromen-4-yl]-N′-(1-methyl-1H-indazol-4-yl)urea

### Example 61A

### 2,2-Dipropyl-8-fluorochroman-4-one

The title compound was prepared according to the procedure of Example 26A, substituting 4-heptanone for 3-pentanone. MS (DCl/NH₃) m/z 268 (M+NH₄)⁺.

### Example 61B

### (R)-6-Fluoro-2,2-dipropylchroman-4-amine

The title compound was prepared from Example 61A according to the methods described in Example 1B and Example 1C. MS (DCl/NH₃) m/z 252 (M+H)⁺.

### Example 61C

### N-[(4R)-6-Fluoro-2,2-dipropyl-3,4-dihydro-2H-chromen-4-yl]-N′-(1-methyl-1H-indazol-4-yl)urea

The title compound was prepared according to the procedure of Example 1H, substituting Example 61B for Example 1D. ¹H NMR (300 MHz, DMSO-d₆) δ 8.74 (s, 1H), 8.04 (d, J=0.9 Hz, 1H), 7.70 (d, J=7.2 Hz, 1H), 7.27 (d, J=7.7 Hz, 1H), 7.09-6.96 (m, 3H), 6.81-6.74 (m, 2H), 4.99-4.90 (m, 1H), 4.01 (s, 3H), 2.18 (dd, J=13.4, 6.1 Hz, 1H), 1.78-1.26 (m, 9H), 0.94-0.85 (m, 6H); MS (DCl/NH₃) m/z 425 (M+H)⁺; [α]²³_{D}+15° (c 0.62, CH₃OH).

### Example 62

### N-[(4R)-2,2-Diethyl-8-fluoro-3,4-dihydro-2H-chromen-4-yl]-N′-(3-methylisoquinolin-5-yl)urea

The title compound was prepared according to the procedure of Example 5 substituting Example 35B for isoquinoline-5-amine, and substituting Example 50B for Example 1 C. ¹H NMR (300 MHz, DMSO-d₆) δ 9.18 (s, 1H), 8.64 (s, 1H), 8.29 (dd, J=7.7, 1.1 Hz, 1H), 7.78-7.68 (m, 2H), 7.53 (t, J=7.9 Hz, 1H), 7.13 (dd, J=20.6, 9.4 Hz, 2H), 7.00 (d, J=8.3 Hz, 1H), 6.89 (td, J=8.0, 5.0 Hz, 1H), 5.03 (s, 1H), 2.65 (s, 3H), 2.24 (dd, J=13.6, 6.1 Hz, 1H), 1.85-1.53 (m, 5H), 0.98-0.81 (m, 6H); MS (ESI) m/z 408 (M+H)⁺.

### Example 63

### N-1H-Indazol-4-yl-N′-[(4R)-8-(trifluoromethyl)-3,4-dihydro-2H-chromen-4-yl]urea

The title compound was prepared according to the procedure of Example 2D, substituting Example 59B for Example 1D. ¹H NMR (300 MHz, DMSO-d₆) δ 13.01 (s, 1H), 8.58 (s, 1H), 8.03 (s, 1H), 7.67 (d, J=7.3 Hz, 1H), 7.62 (d, J=7.6 Hz, 1H), 7.55 (d, J=7.8 Hz, 1H), 7.28-7.16 (m, 1H), 7.13-7.03 (m, 2H), 5.07-4.91 (m, 1H), 4.52-4.39 (m, 1H), 4.35-4.21 (m, 1H), 2.32-1.97 (m, 2H); MS (DCl/NH₃) m/z 377 (M+H)⁺; [α]₂₃D+83.3° (c 0.61, MeOH).

### Example 64

### N-Isoquinolin-5-yl-N′-[(4R)-8-(trifluoromethyl)-3,4-dihydro-2H-chromen-4-yl]urea

The title compound was prepared according to the procedure of Example 5 substituting Example 59B for Example 1C. ¹H NMR (300 MHz, DMSO-d₆) δ 9.28 (s, 1H), 8.60 (s, 1H), 8.54 (d, J=6.0 Hz, 1H), 8.35 (dd, J=7.7, 0.8 Hz, 1H), 7.88 (d, J=6.1 Hz, 1H), 7.76 (d, J=8.2 Hz, 1H), 7.68-7.51 (m, 3H), 7.21 (d, J=7.7 Hz, 1H), 7.08 (app t, J=7.7 Hz, 1H), 5.10-4.91 (m, 1H), 4.54-4.40 (m, 1H), 4.38-4.22 (m, 1H), 2.31-2.01 (m, 2H); MS (DCl/NH₃) m/z 388 (M+H)⁺; [α]²³_{D}+78.9° (c 0.55, 1:1 CH₂Cl₂-MeOH).

### Example 65

### (R)-1-[6-fluoro-2,2-bis(fluoromethyl)chroman-4-yl]-3-(3-methylisoquinolin-5-yl)urea

### Example 65A

### 6-fluoro-2,2-bis(fluoromethyl)chroman-4-one

The title compound was prepared according to the procedure of Example 1A, using 1-(5-fluoro-2-hydroxyphenyl)ethanone and substituting 1,3-difluoropropan-2-one for propan-2-one. MS (DCl) m/z 248 (M+NH₄)⁺.

### Example 65B

### (S)-6-fluoro-2,2-bis(fluoromethyl)chroman-4-ol

The title compound was prepared according to the procedure of Example 1B, substituting Example 65A for Example 1A.

MS (DCl) m/z 232 (M+H)⁺.

### Example 65C

### (R)-6-fluoro-2,2-bis(fluoromethyl)chroman-4-amine

A solution of Example 65B (2.60 g, 11.2 mmol) in THF (52 mL) was cooled to <5° C. To this solution was added 1,8-diazabicyclo[5.4.0]undec-7-ene (2.51 mL, 16.8 mmol) followed by diphenylphosphoryl azide (3.14 mL, 14.6 mmol), keeping the temperature <5° C. (no exotherm). After 2h at ≦5° C., the reaction was warmed to ambient temperature and stirred for 14 h, at which time LCMS indicated complete reactionc. The reaction was diluted with MTBE (70 mL), washed with 2N NaOH (30 mL), brine, 2N HCl (30 mL), and brine (25 mL). The organic portion was dried (Na₂SO₄) and concentrated. The resulting residue was purified by silica gel chromatography (gradient elution, 0%-20% EtOAc/hexanes) to obtain (R)-4-azido-6-fluoro-2,2-bis(fluoromethyl)chroman (2.34 g, 9.10 mmol, 81% yield).

The (R)-4-azido-6-fluoro-2,2-bis(fluoromethyl)chroman (2.33 g, 9.06 mmol) pre-pared above and solvent MeOH (50 mL) were added to 5% Pd—C (699 mg) in a 250 mL stainless steel pressure bottle and stirred for 3 h at 50° C. and 30 psi. The mixture was filtered through a nylon membrane used without further purification.

MS (DCl) m/z 232 (M+H)⁺.

### Example 65D

### (R)-6-fluoro-2,2-bis(fluoromethyl)chroman-4-amine, D-tartaric acid salt

Example 65C (2.09 g, 9.06 mmol) was dissolved in MeOH (20 mL) and D-(−)-tartaric acid (1.36 g, 9.06 mmol) was added. No solids formed, so added MTBE (40 mL) was added. The solution was cooled to 0° C., isopropanol (20 mL), and stirring was continued for 48 h. Solids that formed were filtered and washed with IPA. The resulting solid was dried in a vacuum oven at 60° C., giving Example 65D (2.94 g, 7.71 mmol, 85% yield).

MS (DCl) m/z 232 (M+H)⁺.

### Example 65E

### (R)-1-[6-fluoro-2,2-bis(fluoromethyl)chroman-4-yl]-3-(3-methylisoquinolin-5-yl)urea

A slurry of 3-methylisoquinolin-5-amine (0.498 g, 3.15 mmol) in dichloromethane (10 mL), and pyridine (0.255 mL, 3.15 mmol) was cooled to 5° C. and phenyl chloroformate (0.395 mL, 3.15 mmol) was added dropwise. The light yellow slurry was stirred at 5° C. After 10 min, diisopropylethylamine (1.83 mL, 10.5 mmol) and Example 65D (1.00 g, 2.62 mmol) was added. The solution was warmed to ambient temperature and stirred for 2.5 h. The reaction mixture was diluted with EtOAc (25 mL) and washed with 2N HCl (2×15 mL), brine (20 mL), 2N NaOH (2×15 mL), and brine (20 mL). The organic portion was dried (Na₂SO₄), concentrated, and the resulting residue was purified by silica gel chromatography (gradient elution, 0-10% MeOH/DCM, then 50-100% EtOAc/hexanes) to give the title compound (758 mg, 1.825 mmol, 69.6% yield) as an off-white solid. ¹H NMR (300 MHz, DMSO-d₆) δ ppm 9.19 (s, 1H), 8.66 (s, 1H), 8.25 (d, J=7.5 Hz, 1H), 7.75 (s, 1H), 7.74 (d, J=9.5 Hz, 1H), 7.53 (t, J=7.9 Hz, 1H), 7.2-6.9 (m, 4H), 5.1-5.0 (m, 1H), 4.8-4.5 (m, 4H), 2.66 (s, 3H), 2.35 (dd, J=13.5, 6.0 Hz, 1H), 1.99 (dd, J=13.5, 2.0 Hz, 1H); MS (DCl/NH₃) m/z 416 (M+H)⁺; [α]²³_{D}+8.1°(c 0.57, CH₃OH).

### Example 66

### (R)-1-(3-methylisoquinolin-5-yl)-3-[8-(trifluoromethoxy)chroman-4-yl]urea

### Example 66A

### 1-(prop-2-ynyloxy)-2-(trifluoromethoxy)benzene

To a solution of 2-trifluoromethoxyphenol (10.0 g, 56.1 mmol) in acetonitrile (120 mL) was added potassium carbonate (9.31 g, 67.4 mmol) and propargyl bromide (80% in toluene, 10.0 g, 7.70 mL, 67.4 mmol). The reaction was stirred at ambient temperature for seven days, then diluted with water (150 mL) and extracted with diethyl ether (300 mL). The organic layer was separated and concentrated to obtain the desired product (13.05 g) which was used without further purification in the next step.

¹H NMR (300 MHz, CDCl₃) δ 7.30-7.23 (m, 2H), 7.19-7.13 (m, 1H), 7.04-6.95 (m, 1H), 4.77 (d, J=2.4 Hz, 2H), 2.53 (t, J=2.4 Hz, 1H).

### Example 66B

### 1-(3-chloroprop-2-ynyloxy)-2-(trifluoromethoxy)benzene

To a solution of the product of Example 66A (13.0 g, 56.1 mmol) in acetone (200 mL) was added N-chlorosuccinimide (8.99 g, 67.3 mmol) and silver acetate (0.936 g, 5.61 mmol). The reaction was heated to reflux for 16 h, cooled to ambient temperature, and the solvent removed under reduced pressure. The residue was taken up in a mixture of diethyl ether and water, and filtered to remove the silver salts. The filtrate was extracted with diethyl ether (300 mL). The combined organic layers were washed with saturated sodium bicarbonate (75 mL) and concentrated to give the title compound (12.85 g) which was used without further purification in the next step.

¹H NMR (300 MHz, CDCl₃) δ 7.30-7.24 (m, 2H), 7.15-7.09 (m, 1H), 7.01 (td, J=7.8, 1.4 Hz, 1H), 4.77 (s, 2H); MS (DCl) m/z 268 (M+NH₄)⁺.

### Example 66C

### 8-(trifluoromethoxy)chroman-4-one

A solution of the product of Example 66B (12.8 g, 51.2 mmol) in ethylene glycol (200 mL) was heated to reflux for 6 hours, cooled to ambient temperature and stirred for 16 h, then heated to reflux for an additional 3 hours. After cooling, the reaction mixture was poured into water (100 mL) and extracted with diethyl ether (250 mL). The mixture was partitioned and the organic portion was concentrated. The resulting residue was purified by silica gel chromatography (gradient elution, 0%-20% EtOAc/hexanes) to obtain the title compound (3.62 g, 28% for three steps). ¹H NMR (300 MHz, CDCl₃) δ 7.86 (dd, J=8.1, 1.7 Hz, 1H), 7.44 (d, J=7.8 Hz, 1H), 7.05-6.98 (m, 1H), 4.66-4.60 (m, 2H), 2.90-2.84 (m, 2H).

### Example 66D

### (S)-8-(trifluoromethoxy)chroman-4-ol

The title compound was prepared according to the procedure of Example 1B, substituting Example 66C for Example 1A. ¹H NMR (300 MHz, DMSO-d₆) δ 7.42-7.12 (m, 2H), 6.98-6.89 (m, 1H), 5.52 (d, J=5.4 Hz, 1H), 4.72-4.61 (m, 1H), 4.35-4.19 (m, 2H), 2.11-1.96 (m, 1H), 1.95-1.83 (m, 1H); MS (DCl) m/z 217 (M−H₂O)⁺.

### Example 66E

### (R)-8-(trifluoromethoxy)chroman-4-amine

The title compound was prepared according to the procedure of Example 1C, substituting Example 66D for Example 1B. ¹H NMR (300 MHz, DMSO-d₆) δ 7.41 (d, J=7.4 Hz, 1H), 7.15 (d, J=8.2 Hz, 1H), 6.96-6.84 (m, 1H), 4.39-4.15 (m, 2H), 3.92 (t, J=5.5 Hz, 1H), 2.10-1.87 (m, 3H), 1.83-1.67 (m, 1H); MS (DCl) m/z 234 (M+H)⁺.

### Example 66F

### (R)-8-(trifluoromethoxy)chroman-4-amine, D-tartaric acid salt

The title compound was prepared according to the procedure of Example 65D, substituting Example 66E for Example 65C. ¹H NMR (300 MHz, DMSO) δ 7.46 (d, J=7.9 Hz, 1H), 7.32 (d, J=8.1 Hz, 1H), 7.13-6.95 (m, 1H), 4.50-4.24 (m, 2H), 3.94 (s, 2H), 2.30-2.13 (m, 1H), 2.09-1.87 (m, 1H); MS (DCl) m/z 234 (M+H)⁺.

### Example 66G

### (R)-1-(3-methylisoquinolin-5-yl)-3-[8-(trifluoromethoxy)chroman-4-yl]urea

A suspension of 3-methylisoquinolin-5-amine (0.263 g, 1.66 mmol) and pyridine (0.134 mL, 1.66 mmol) in dichloromethane (6 mL) was cooled in an ice bath. A solution of phenyl chloroformate (0.260 g, 0.209 mL, 1.66 mmol) in dichloromethane (1 mL) was added slowly, and the reaction allowed to stir for 10 min before adding N,N-diisopropylethylamine (0.715 g, 0.966 mL, 5.53 mmol). The product of Example 66G (0.530 g, 1.38 mmol) was added, and the reaction allowed to stir at 0° C. for 1 h and then at ambient temperature for 16 h. The reaction mixture was diluted with dichloromethane (10 mL), 1 N aqueous sodium hydroxide (5 mL) was added and the precipitate filtered. The filtrate was treated with additional of 1 N NaOH (5 mL) and more of the precipitate was collected by filtration. The solids were combined, titurated with water, collected by filtration, and dried to give the title compound (298 mg, 52%). ¹H NMR (300 MHz, DMSO) δ 9.17 (s, 1H), 8.51 (s, 1H), 8.31 (d, J=7.0 Hz, 1H), 7.74-7.66 (m, 2H), 7.57-7.48 (m, 1H), 7.38 (d, J=7.8 Hz, 1H), 7.29 (d, J=8.1 Hz, 1H), 7.18 (d, J=7.7 Hz, 1H), 7.05-6.96 (m, 1H), 5.05-4.95 (m, 1H), 4.49-4.38 (m, 1H), 4.32-4.21 (m, 1H), 2.63 (s, 3H), 2.26-2.01 (m, 2H); MS (DCl) m/Z₄₁₈ (M+H)⁺; [α]²³_{D}=+ 49.6° (c=0.50, 1:1 MeOH—CH₂Cl₂).

Other compounds were prepared using similar methodology as described above. Additional compounds include the following:
N-(3-methylisoquinolin-5-yl)-N-[(4R)-8-(trifluoromethoxy)-3,4-dihydro-2H-chromen-4-yl]urea;
N-[(4R)-8-fluoro-2,2-dipropyl-3,4-dihydro-2H-chromen-4-yl]-N′-(1-methyl-1H-indazol-4-yl)urea;
N-[(4R)-6-fluoro-2,2-bis(fluoromethyl)-3,4-dihydro-2H-chromen-4-yl]-N-(3-methylisoquinolin-5-yl)urea;
N-[(4R)-8-fluoro-2,2-dipropyl-3,4-dihydro-2H-chromen-4-yl]-/V-(3-methylisoquinolin-5-yl)urea;
N-[(4R)-7-chloro-2,2-diethyl-3,4-dihydro-2H-chromen-4-yl]-N-(3-methylisoquinolin-5-yl)urea;
N-1H-indazol-4-yl-/V-[(4R)-8-(trifluoromethoxy)-3,4-dihydro-2H-chromen-4-yl]urea;
N-[(4R)-2,2-diethyl-7-fluoro-3,4-dihydro-2H-chromen-4-yl]-N-[(7S)-7-hydroxy-5,6,7,8-tetrahydronaphthalen-1-yl]urea;
N-[(4R)-6-fluoro-2,2-dipropyl-3,4-dihydro-2H-chromen-4-yl]-/V-[(7R)-7-hydroxy-5,6,7,8-tetrahydronaphthalen-1-yl]urea;
N-[(4R)-2,2-diethyl-6-fluoro-3,4-dihydro-2H-chromen-4-yl]-N-[(7S)-7-hydroxy-5,6,7,8-tetrahydronaphthalen-1-yl]urea;
N-[(4R)-2,2-diethyl-7-fluoro-3,4-dihydro-2H-chromen-4-yl]-N-[(7R)-7-hydroxy-5,6,7,8-tetrahydronaphthalen-1-yl]urea;
N-[(4R)-7-chloro-2,2-diethyl-3,4-dihydro-2H-chromen-4-yl]-N-[(7R)-7-hydroxy-5,6,7,8-tetrahydronaphthalen-1-yl]urea; and
N-[(4R)-7-chloro-2,2-diethyl-3,4-dihydro-2H-chromen-4-yl]-N-[(7S)-7-hydroxy-5,6,7,8-tetrahydronaphthalen-1-yl]urea.
   B. Preparation of Solid Dispersion Products and Evaluation thereof.

### Example 1

### Preparation of ABT 102 Solid Dispersion Products

Solid dispersion products wherein the matrix-forming agent is PVP are prepared according to the following protocol:
(1) Dissolve PVP in ethanol. For PVP K30 prepare a 30% (w/w) solution, for PVP K12 prepare a 50% (w/w) solution.
(2) Melt surfactants at 60° C. in an oven and mix in the ratio indicated.
(3) Weigh PVP solution into amber glass bottle.
(4) Weigh active agent (ABT 102) and add to PVP solution; stir until dissolved.
(5) Add surfactant and mix. If surfactant solidifies partially, warm again.
(6) If solution is still turbid after one hour, add further ethanol and homogenize.

Solid dispersion products wherein the matrix-forming agent is hydroxypropyl-p-cyclodextrin (HP-β-CD) are prepared according to the following protocol:

(1) Weigh 8.5 g HP-β-CD and dissolve in 60 g ethanol (anhydrous).
(2) Weigh active agent and dissolve in (1).
(3) Melt surfactant and add to (2).
(4) If surfactant solidifies partially, warm again until a clear solution is obtained.

Spray drying was performed using a Büchi B-191 lab scale spray dryer. The equipment was pre-heated before the spray cycle was started. After spraying a final drying was conducted for 10-20 minutes before the cooling cycle was initiated. For atomization of the liquid a two-component nozzle (liquid plus air for atomization) has been used.

### Protocol For The Oral Bioavailability Studies

For bioavailability evaluation, solid dispersion powder as obtained in example were screened and filled into capsules or compressed to tablets. Each capsule contained 16.7 mg ABT 102, tablets contained 50 mg ABT-102.

The studies were run in a randomized cross-over study design.

Dogs (beagle dogs, mixed sexes, weighing approximately 10 kg) were fasted overnight prior to dosing, but were permitted water ad libitum; food was provided to the dogs about 30 minutes prior to dosing. A single dose corresponding to 25-50 mg ABT 102 was administered to each dog. The dose was followed by approximately 10 milliliters of water. Blood samples were obtained from each animal prior to dosing and 0.25, 0.5, 1.0, 1.5, 2, 3, 4, 6, 9, 12,15 and 24 hours after drug administration. The plasma was separated from the red cells by centrifugation and frozen (−20° C.) until analysis. Concentrations of ABT 102 were determined by reverse phase HPLC with HPLC-MS/MS quantitation following liquid-liquid extraction of the plasma samples. The area under the curve (AUC) was calculated by the trapezoidal method over the time course of the study. Each dosage form was evaluated in a group containing 3-6 dogs; the values reported are averages for each group of dogs.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

TABLE 1
Solid Dispersion Product Composition and absolute Bioavailability in Dog

| | Example | | | | | | | |
|---|---|---|---|---|---|---|---|---|
| | 1-1 | 1-2 | 1-3 | 1-4 | 1-5 | 1-6 | 1-7 | 1-8 |
| ABT 102 | 6.67 | 5.71 | 6.67 | 5.71 | 5.71 | 5.71 | 2.10 | 4.32 |
| Kollidon K12 | 33.33 | 28.57 | 33.33 | 28.57 | 28.57 | 28.57 | — | — |
| Kollidon K30 | — | — | — | — | — | — | — | 21.62 |
| HP-β-CD | — | — | — | — | — | — | 11.89 | — |
| Cremophor RH40 | 6.67 | 9.29 | — | — | 27.86 | — | — | — |
| TPGS | — | — | 11.19 | 15.59 | — | 15.64 | 2.10 | 11.83 |
| Gelucire 44/14 | 20.00 | 27.86 | 15.47 | 21.55 | 9.29 | 21.51 | - | 16.27 |
| Ethanol | 33.33 | 28.57 | 33.33 | 28.57 | 28.57 | 28.57 | 83.92 | 45.95 |
| Total w/o Ethanol | 66.67 | 71.43 | 66.67 | 71.43 | 71.43 | 71.43 | 16.08 | 54.05 |
| Total | 100.00 | 100.00 | 100.00 | 100.00 | 100.00 | 100.00 | 100.00 | 100.00 |
| Drug load | 10.00 | 8.00 | 10.00 | 8.00 | 8.00 | 8.00 | 13.04 | 8.00 |
| F (dog), fasted | 4.8 | 14.4 | 8.2 | 18.4 | 9.5 | 29.2 | 6.8 | 28.2 |
| F (dog), non-fasted | n.d. | n.d. | n.d. | n.d. | n.d. | 73.0 | n.d. | 73.8 |

n.d. = not determined
F = estimated absolute bioavailability
Kollidon K12 = PVP K12
Kollidon K30 = PVP K30
HP-β-CD = Hydroxypropyl-β-cyclodextrin
Cremophor RH40 = polyoxyethylene glycerol trihydroxystearate 40
TPGS = Vitamin E TPGS 1000
Gelucire 44/14 = lauroyl macrogol 32 glycerides

### Example 2

Following the procedures of Example 1 above, a liquid mixture is prepared, containing 56.13% by weight of ethanol, 15.36% of PVP K30, 3.56% of Gelucire 44/14, 1.92% of Vitamin E TPGS, 21.94% of maltitol and 1.10% of ABT-102.

The liquid mixture is fed to a twin-drum dryer. This dryer comprises a pair of drums which are rotated in the opposite direction to each other. The drums are heated to a temperature of about 60° C. by circulating thermal oil. The space between the drums forms a liquid pool into which the liquid mixture is introduced. The liquid mixture is being spread on the circumferential faces of the respective drums; the adjustable gap between the two drums acts as a means to control the film thickness. After travelling part of a revolution, the dried material is removed in the form of thin sheets by scraper knifes.

The drying drums are positioned in a vacuum chamber which is maintained at a pressure of 50 mbar (absolute pressure). The ethanol vapours are drawn off and condensed.

### Example 3

Following the procedures of Example 1 above, a spray-dried solid dispersion product was obtained, having a composition of ABT-102: Kollidon K30: Gelucire 44/14: Vitamin

E TPGS (2.4:33.6:7.8:4.2; % by weight). The spray-dried formulation (48.0 parts by weight) was blended with Isomalt (48.0 parts by weight), Aerosil 200 (1.0 parts by weight) and sodium stearyl fumarate (3.0 parts by weight). The mixture was filled into hard gelatine capsules or compacted to tablets, each containing 12.5 mg ABT 102.

The formulations were administered at a dose of 25 mg/dog. Each dog received 2×12.5 mg experimental capsules or tablets. The results are shown in Table 2 below:

TABLE 2
Plasma Concentration following a 25 mg Oral Dose in Dog

| | | Cₘₐₓ | | AUC |
|---|---|---|---|---|
| Form | t_{1/2} [hr]* | [μg/mL] | Tₘₐₓ [hr] | [μg · hr/mL] |
| Capsule | 3.0 | 0.17 (0.06) | 6.3 (1.9) | 1.07 (0.30) |
| Tablet | 2.7 | 0.37 (0.08) | 5.7 (2.3) | 2.94 (0.76) |

*harmonic mean; mean (SEM, n = 6)

### Example 4

Following the procedures of Example 1 above, a spray-dried solid dispersion product was obtained, having a composition of ABT-102: Kollidon K30: Gelucire 44/14: Vitamin E TPGS (5.02:69.99:16.24:8.75; % by weight).

A study was conducted to explore the ABT-102 plasma concentrations following multiple oral dosing in rat. In this study, a 10, 30 or 100 mg/kg/day oral dose was administered once daily for eight consecutive days. The compound was prepared as a suspension of the spray dried material in water at concentrations appropriate for a 20 ml/kg/day dose volume in each treatment group.

The study was conducted in Sprague-Dawley rats (3 male, 3 female per dose group). Animals were permitted free access to food and water throughout the study. Plasma concentrations of parent drug were determined on the first (Day 1) and last (Day 8) of dosing. The results are shown in Table 3 below:

TABLE 3
Plasma Concentration following Multiple Oral Dosing in Rat

| Dose | Day | t_{1/2} | Cₘₐₓ | Cₘₐₓ/D | Tₘₐₓ | AUC | AUC/D |
|---|---|---|---|---|---|---|---|
| 10 | 1 | 5.9⁰ | 0.73 | 0.073 | 2.3 | 9.18 | 0.918 |
| | | | (0.12) | | (0.3) | (1.37) | |
| | 8 | 5.4⁰ | 0.60 | 0.060 | 3.3 | 7.25 | 0.725 |
| | | | (0.06) | | (0.6) | (0.64) | |
| 30 | 1 | 7.8⁰ | 1.39 | 0.046 | 3.0 | 22.22 | 0.741 |
| | | | (0.18) | | (0.0) | (4.39) | |
| | 8 | 5.3⁰ | 1.32 | 0.044 | 3.3 | 14.95 | 0.498 |
| | | | (0.28) | | (0.6) | (1.53) | |
| 100 | 1 | 6.3⁰ | 2.13 | 0.021 | 5.0 | 32.61 | 0.326 |
| | | | (0.16) | | (1.0) | (4.54) | |
| | 8 | 5.6⁰ | 2.61 | 0.026 | 4.5 | 36.18 | 0.362 |
| | | | (0.20) | | (0.7) | (4.02) | |

⁰harmonic mean;
t_{1/2} [hr];
Cₘₐₓ [μg/mL];
Tₘₐₓ [hr];
AUC [μg · hr/mL];
AUC/D [μg · hr/mL per mg/kg];
Cₘₐₓ/D [μg/mL per mg/kg];
mean (SEM);

Peak plasma concentrations following the 10, 30 or 100 mg/kg doses averaged 0.73, 1.39 and 2.13 μg/ml, respectively; Cₘₐₓ values at the end of the study were comparable to those measured on Day 1, averaging 0.60, 1.32 and 2.61 μg/ml in the same treatment groups. AUC values averaged 9.2, 22.2 and 32.6 μg·hr/ml on the first day of the study, remaining constant at 7.3, 15 and 36.2 μg·hr/ml on Day 8.

### Example 5

Following the procedures of Example 1 above, a spray-dried solid dispersion product was obtained, having a composition of ABT-102: Kollidon K30: Gelucire 44/14: Vitamin E TPGS (6.0:58.0:23.4:12.6; % by weight)

A study was conducted to evaluate effect of aging on the ABT-102 bioavailability obtained from suspensions of the spray dried material. Suspensions were prepared by stirring in water for 15 minutes at room temperature (5 mg/ml concentration). The suspensions were then stored refrigerated until dosing. Suspensions aged for 1, 4 and 7 days were compared to a suspension freshly prepared on the morning of dosing. Each of the aged suspension was evaluated in a group of three rats at a dose of 100 mg/kg (20 ml/kg). All four test formulations were evaluated in the same study. Plasma concentrations of parent drug were determined by HPLC-MS/MS.

TABLE 4
Plasma Concentrations following a 100 mg/kg Oral Dose in Rat

| | | | % Day 0 | | | % Day 0 |
|---|---|---|---|---|---|---|
| Days aged | t_{1/2} | Cₘₐₓ | (Cₘₐₓ) | Tₘₐₓ | AUC | (AUC) |
| 7 | 4.4⁰ | 1.16 (0.18) | 82 | 4.3 (2.3) | 12.37 (1.91) | 72 |
| 4 | 3.8⁰ | 1.62 (0.22) | 114 | 3.7 (1.2) | 20.04 (1.93) | 117 |
| 1 | 3.8⁰ | 1.47 (0.12) | 104 | 4.3 (2.3) | 19.17 (2.83) | 112 |
| 0 | 5.4⁰ | 1.42 (0.30) | 100 | 3.3 (1.3) | 17.14 (5.30) | 100 |

⁰harmonic mean;
t_{1/2} [hr];
Cₘₐₓ [μg/mL];
Tₘₐₓ [hr];
AUC [μg · hr/mL];
mean (SEM);

Peak plasma concentrations and AUC values obtained from the suspensions aged for 1 or 4 days prior to dosing were comparable to or slightly higher than values obtained from the freshly prepared suspension. However, plasma concentrations obtained from suspensions prepared 7 days prior to dosing were ˜30% lower than those obtained from the freshly prepared suspension. The results from this study suggest that suspensions prepared every three to four days will provide comparable plasma concentrations after oral dosing in rat to those obtained from freshly prepared suspensions.

### Example 6

### Physical Stability Determination

The physical stability of solid dispersion products stored at stressed condition was monitored. Powder X-ray diffraction patterns (PXRD) were recorded to detect crystallization of ABT-102, if any.

PXRD data were collected using a G3000 diffractometer (Inel Corp., Artenay, France) equipped with a curved position sensitive detector and parallel beam optics. The diffractometer was operated with a copper anode tube (1.5 kW fine focus) at 40 kV and 30 mA. An incident beam germanium monochromator provided monochromatic Kα1 radiation. The diffractometer was calibrated using the attenuated direct beam at one-degree intervals. Calibration was checked using a silicon powder line position reference standard (NIST 640c). The instrument was computer controlled using the Symphonix software (Inel Corp., Artenay, France) and the data was analyzed using the Jade software (version 6.5, Materials Data, Inc., Livermore, Calif.). The sample was loaded onto an aluminum sample holder and leveled with a glass slide.

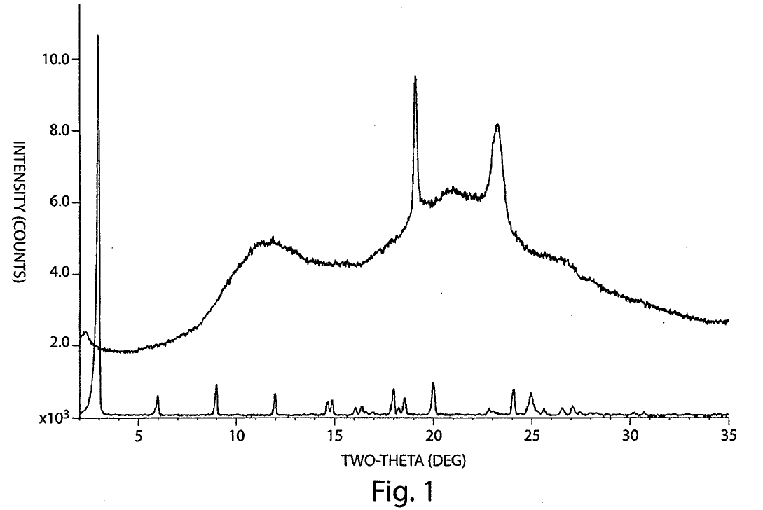

PXRD pattern of an excipient mixture containing Kollidon-30, Gelucire 44/14, and Vitamin E-TPGS show a smooth halo due to the disorderness of each component (FIG. 1, above). Crystalline ABT-102 has a unique and intense diffraction peak at 2.9°/2θ (FIG. 1, bottom). This diffraction peak can be used to identify the existence of crystalline ABT-102.

Spray-dried solid dispersions of ABT-102 with various drug load (25% and 15%) and polymers were prepared from methanol (Table 5). The weight loss was measured to be 0.2% to 8.4% (w/w) when the solids were heated above 100° C. The weight loss was mainly due to the residual solvent, methanol.

TABLE 5
Spray-dried solid dispersion stored at 40° C./75% RH

| Example | ABT-102 | Polymer | Residual Solvent |
|---|---|---|---|
| 6-1 | 25% | HPMC-AS | 1.7 |
| 6-2 | 25% | PVP-VA64 | 8.4 |
| 6-3 | 25% | Kollidon 29/32 | 4.5 |
| 6-4 | 25% | HPMC-E5 | 3 |
| 6-5 | 15% | HPMC-AS | 1.7 |
| 6-6 | 15% | PVP-VA64 | 0.2 |

HPMC-AS = hydroxypropylmethylcellulose acetate succinate
PVP-VA64 = copolymer of N-vinyl pyrrolidone and vinyl acetate 60/40% by weight
Kollidon 29/32 = PVP K29-32
HPMC-E5 = hydroxypropylmethylcellulose, molecular weight of 5,000
HPMC-AS = hydroxypropylmethylcellulose acetate succinate

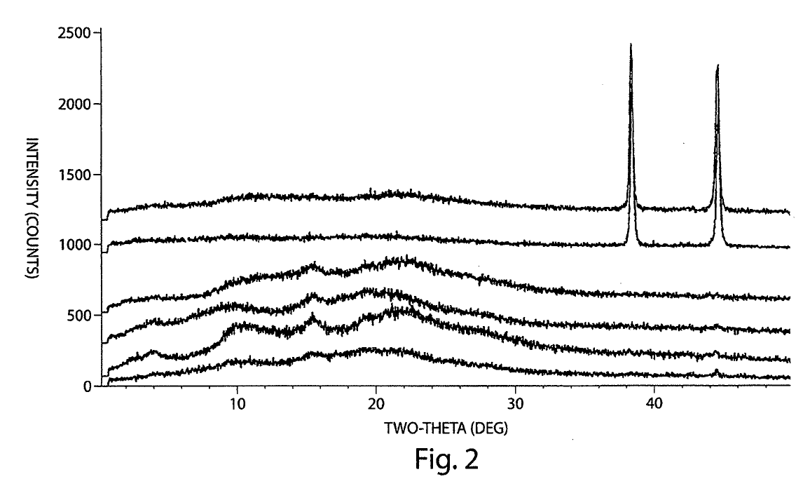

The solids were stored at 40° C./75% RH (relative humidity) stability chamber. FIG. 2 shows (from the bottom up) PXRDs of Example 6-1; 6-3; 6-4; 6-2, stored for 6 weeks; and Example 6-5 and 6-6, stored for 4 weeks. No significant crystallization was observed in the solid dispersion formulations containing 25% and 15% (w/w) ABT-102 up to 6 and 4 weeks, respectively.

As shown in Example 3 above, ABT-102 dosage forms of the invention provide Cₘₐₓ values ranging from 0.17 to 0.37 μg/ml and AUC values ranging from 1.07 to 2.94 μg.hr/ml in dogs, following a 25 mg dose of ABT-102.

Based on previously conducted human pharmacokinetic data for ABT-102, it was determined that pharmacokinetics of ABT-102 was characterized by dose proportional exposures (Cmax and AUC). This data was generated using a lipid-liquid formulation. However, it is anticipated that the current spray dried formulation of the invention also achieves similar pharmacokinetic profile in humans.

The invention therefore contemplates ABT-102 oral dosage forms wherein a single-dose administration provides in a patient a blood plasma level profile with a dosage-corrected Cmax between 0.8 and 2.4 ng/ml*mg, wherein said dosage-corrected Cmax is Cmax divided by the number of milligrams of ABT-102 in the dosage form.

The invention further contemplates ABT-102 oral dosage forms, having a dosage-corrected AUC_{∞} between 18 and 35 ng.h/ml*mg, wherein said dosage-corrected AUC_{∞} is the AUC_{∞} divided by the number of milligrams of ABT-102 in the dosage form following single dose administration.

### Incorporation by Reference

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

We claim:

1. A solid dispersion product comprising at least one pharmaceutically active agent, obtained by
a) preparing a liquid mixture containing the at least one active agent, at least one pharmaceutically acceptable matrix-forming agent, at least one pharmaceutically acceptable surfactant and at least one solvent, and
b) removing the solvent(s) from the liquid mixture to obtain the solid dispersion product.

2. The solid dispersion product of claim 1, wherein the active agent is a N-aryl urea-based active agent.

3. The solid dispersion product of claim 1, wherein at least one filler is added to the liquid mixture before removing the solvent(s).

4. The solid dispersion product of claim 1, wherein the mass ratio of active agent and pharmaceutically acceptable matrix-forming agent is from 0.01:1 to 1:3.

5. The solid dispersion product of claim 1, wherein the mass ratio of active agent and pharmaceutically acceptable surfactant is from 0.1:1 to 1:7.

6. The solid dispersion product of claim 1, wherein the pharmaceutically acceptable matrix-forming agent is selected from the group consisting of cyclodextrines, pharmaceutically acceptable polymers, lipids or combinations of two or more thereof.

7. The solid dispersion product of claim 1, wherein said pharmaceutically acceptable matrix-forming agent is selected from the group consisting of cellulose esters, cellulose ethers, cellulose ether-esters, maltodextrines, N-vinyl pyrrolidone homopolymers, N-vinyl pyrrolidone copolymers and combinations of two or more thereof.

8. The solid dispersion product of claim 1, wherein said pharmaceutically acceptable matrix-forming agent is selected from the group consisting of poly N-vinylpyrrolidones, copolymers of N-vinyl pyrrolidone and vinyl acetate and combinations thereof.

9. The solid dispersion product of claim 1, wherein the pharmaceutically acceptable surfactant is selected from the group consisting of polyol fatty acid esters, polyalkoxylated polyol fatty acid esters, polyalkoxylated fatty alcohol ethers, tocopheryl compounds or combinations of two or more thereof.

10. The solid dispersion product of claim 1, wherein the pharmaceutically acceptable surfactant comprises a combination of two or more pharmaceutically acceptable surfactants.

11. The solid dispersion product dispersion product of claim 1, wherein the pharmaceutically acceptable surfactant comprises at least one surfactant having an HLB value of 10 or more.

12. The solid dispersion product of claim 10 wherein the combination of pharmaceutically acceptable surfactants comprises (i) at least one tocopheryl compound having a polyalkylene glycol moiety and (ii) at least one polyalkoxylated polyol fatty acid ester.

13. The solid dispersion product of claim 12, wherein the tocopheryl compound is alpha tocopheryl polyethylene glycol succinate.

14. The solid dispersion product of claim 12, wherein the polyalkoxylated polyol fatty acid ester is a polyalkoxylated glyceride.

15. The solid dispersion product of claim 12, wherein the mass ratio of tocopheryl compound and polyalkoxylated polyol fatty acid ester is in the range of from 0.2:1 to 1:1.

16. The solid dispersion product of claim 1, wherein the active agent is represented by the general formula (I) 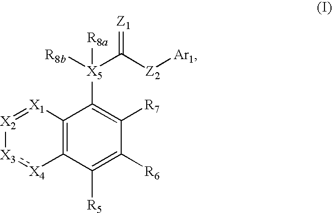
or a pharmaceutically acceptable salt or prodrug thereof, wherein
--- is absent or a single bond;
X₁ is N or CR₁;
X₂ is N or CR₂;
X₃ is N, NR₃, or CR₃;
X₄ is a bond, N, or CR₄;
X₅ is N or C;
provided that at least one of X₁, X₂, X₃, and X₄ is N;
Z₁ is O, NH, or S;
Z₂ is a bond, NH, or 0;
Ar₁is selected from the group consisting of 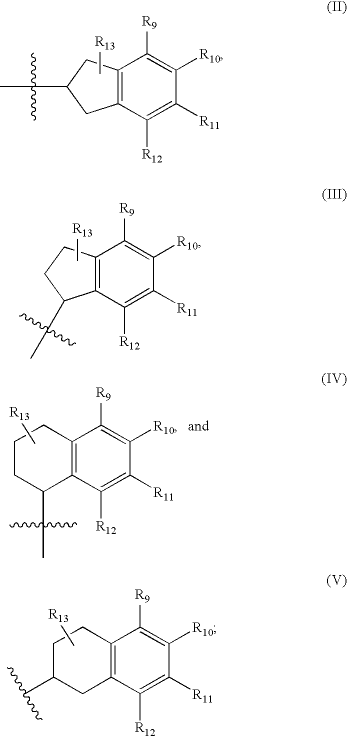
R₁, R₃, R₅, R₆, and R₇ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl;
R₂ and R₄ are each independently selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)alkylcarbonyl, (Z_{A}Z_{B}N)carbonyl, (Z_{A}Z_{B}N)carbonylalkyl, (Z_{A}Z_{B}N)sulfonyl, (Z_{A}Z_{B}N)C(═NH)—, (Z_{A}Z_{B}N)C(═NCN)NH— and (Z_{A}Z_{B}N)C(═NH)NH—;
R₈ₐ is hydrogen or alkyl;
R_{8b} is absent, hydrogen, alkoxy, alkoxycarbonylalkyl, alkyl, alkylcarbonyloxy, alkylsulfonyloxy, halogen, or hydroxy;
R₉, R₁₀, R₁₁, and R₁₂ are each individually selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkynyl, aryl, carboxy, carboxyalkyl, cyano, cyanoalkyl, formyl, formylalkyl, haloalkoxy, haloalkyl, haloalkylthio, halogen, heteroaryl, heterocycle, hydroxy, hydroxyalkyl, mercapto, mercaptoalkyl, nitro, (CF₃)₂(HO)C—, R_{B}(SO)₂R_{A}N—, R_{A}O(SO)₂—, R_{B}O(SO)₂—, Z_{A}Z_{B}N—, (Z_{A}Z_{B}N)alkyl, (Z_{A}Z_{B}N)carbonyl, (Z_{A}Z_{B}N)carbonylalkyl, and (Z_{A}Z_{B}N)sulfonyl, wherein Z_{A} and Z_{B} are each independently hydrogen, alkyl, alkylcarbonyl, formyl, aryl, or arylalkyl, provided that at least one of R₉, R₁₀, R₁₁, or R₁₂ is other than hydrogen, or R₁₀ and R₁₁ taken together with the atoms to which they are attached form a cycloalkyl, cycloalkenyl, or heterocycle ring;
R₁₃ is selected from the group consisting of hydrogen, alkyl, aryl, heteroaryl, and halogen;
R_{A} is hydrogen or alkyl; and
R_{B} is alkyl, aryl, or arylalkyl;
provided that R_{8b} is absent when X₅ is N.

17. The solid dispersion product of claim 1, wherein the active agent is selected from the group consisting of 1-((R)-5-tert-butyl-indan-1-yl)-3-(1H-indazol-4-yl)-urea (ABT102) and salts or hydrates or solvates thereof.

18. The solid dispersion product of claim 1, wherein the active agent is selected from the group consisting of
N-(5-tert-butyl-2,3-dihydro-1H-inden-1-yl)-N′-5-isoquinolinylurea;
N-(5-tert-butyl-2,3-dihydro-1H-inden-1-yl)-N′-(3-methyl-5-isoquinolinyl)urea;
(+) N-(5-tert-butyl-2,3-dihydro-1H-inden-1-yl)-N′-(3-methyl-5-isoquinolinyl)urea;
(−) N-(5-tert-butyl-2,3-dihydro-1H-inden-1-yl)-N′-(3-methyl-5-isoquinolinyl)urea;
(−) N-(5-tert-butyl-2,3-dihydro-1H-inden-1-yl)-N′-5-isoquinolinylurea;
(+) N-(5-tert-butyl-2,3-dihydro-1H-inden-1-yl)-N′-5-isoquinolinylurea;
N-(5-bromo-2,3-dihydro-1H-inden-1-yl)-N′-5-isoquinolinylurea;
methyl 4-({[(5-tert-butyl-2,3-dihydro-1H-inden-1-yl)amino]carbonyl}amino)-1H-indazole-1-carboxylate;
N-(5-tert-butyl-2,3-dihydro-1H-inden-1-yl)-N′-1H-indazol-4-ylurea (ABT-102);
methyl 4-[({[(1 S)-5-tert-butyl-2,3-dihydro-1H-inden-1-yl]amino}carbonyl)amino]-1H-indazole-1-carboxylate;
methyl 4-[({[(1 R)-5-tert-butyl-2,3-dihydro-1H-inden-1-yl]amino}carbonyl)amino]-1H-indazole-1-carboxylate;
N-[(1 S)-5-tert-butyl-2,3-dihydro-1H-inden-1-yl]-N′-1H-indazol-4-ylurea;
N-[(1 R)-5-tert-butyl-2,3-dihydro-1H-inden-1-yl]-N′-1H-indazol-4-ylurea;
methyl 4-[({[5-(trifluoromethyl)-2,3-dihydro-1H-inden-1-yl]amino}carbonyl)amino]-1H-indazole-1-carboxylate;
N-1H-indazol-4-yl-N′-[5-(trifluoromethyl)-2,3-dihydro-1H-inden-1-yl]urea;
methyl 4-({[(5-piperidin-1-yl-2,3-dihydro-1H-inden-1-yl)amino]carbonyl}amino)-1H-indazole-1-carboxylate;
N-1H-indazol-4-yl-N′-(5-piperidin-1-yl-2,3-dihydro-1H-inden-1-yl)urea;
methyl 4-({[(5-hexahydro-1H-azepin-1-yl-2,3-dihydro-1H-inden-1-yl)amino]carbonyl}amino)-1H-indazole-1-carboxylate;
N-(5-hexahydro-1H-azepin-1-yl-2,3-dihydro-1H-inden-1-yl)-N′-1H-indazol-4-ylurea;
N-1H-indazol-4-yl-N′-[(1 R)-5-piperidin-1-yl-2,3-dihydro-1H-inden-1-yl]urea;
N-1H-indazol-4-yl-N′-[(1 S)-5-piperidin-1-yl-2,3-dihydro-1H-inden-1-yl]urea;
isopropyl 4-({[(5-tert-butyl-2,3-dihydro-1H-inden-1-yl)amino]carbonyl}amino)-1H-indazole-1-carboxylate; and
isobutyl 4-({[(5-tert-butyl-2,3-dihydro-1H-inden-1-yl)amino]carbonyl}amino)-1H-indazole-1-carboxylate;
N-[(4R)-6-fluoro-2,2-dimethyl-3,4-dihydro-2H-chromen-4-yl]-N′-(1-methyl-1H-indazol-4-yl)urea;
N-[(4R)-6-fluoro-2,2-dimethyl-3,4-dihydro-2H-chromen-4-yl]-N′-1H-indazol-4-ylurea;
N-[(4R)-6-fluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-6-fluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-6-fluoro-2,2-dimethyl-3,4-dihydro-2H-chromen-4-yl]-N′-isoquinolin-5-ylurea;
N-[(4R)-6-fluoro-3,3′,4,4′-tetrahydro-2′ H-spiro[chromene-2,1′-cyclobutan]-4-yl]-N′-[(7R)-7-hydroxy-5,6,7,8-tetrahydronaphthalen-1-yl]urea;
N-[(4R)-6-fluoro-3,3′,4,4′-tetrahydro-2′ H-spiro[chromene-2,1′-cyclobutan]-4-yl]-N′-(1-methyl-1H-indazol-4-yl)urea;
N-[(4R)-6-fluoro-3,3′,4,4′-tetrahydro-2′ H-spiro[chromene-2,1′-cyclobutan]-4-yl]-N′-1H-indazol-4-ylurea;
N-[(4R)-6-fluoro-3,3′,4,4′-tetrahydro-2′ H-spiro[chromene-2,1′-cyclobutan]-4-yl]-N′-[(7S)-7-hydroxy-5,6,7,8-tetrahydronaphthalen-1-yl]urea;
N-[(4S)-6-fluoro-3,3′,4,4′-tetrahydro-2′ H-spiro[chromene-2,1′-cyclobutan]-4-yl]-N′-[(7S)-7-hydroxy-5,6,7,8-tetrahydronaphthalen-1-yl]urea;
N-[(4S)-6-fluoro-3,3′,4,4′-tetrahydro-2′ H-spiro[chromene-2,1′-cyclobutan]-4-yl]-N′-[(7R)-7-hydroxy-5,6,7,8-tetrahydronaphthalen-1-yl]urea;
N-[(4R)-6-fluoro-3,4-dihydro-2H-chromen-4-yl]-N′-(1-methyl-1H-indazol-4-yl)urea;
N-[(4R)-6-fluoro-3,4-dihydro-2H-chromen-4-yl]-N′-isoquinolin-5-ylurea;
N-[(4R)-6-fluoro-3,4-dihydro-2H-chromen-4-yl]-N′-[(7R)-7-hydroxy-5,6,7,8-tetrahydronaphthalen-1-yl]urea;
N-[(4R)-6-fluoro-3,4-dihydro-2H-chromen-4-yl]-N′-[(7S)-7-hydroxy-5,6,7,8-tetrahydronaphthalen-1-yl]urea;
N-[(4R)-6,8-difluoro-2,2-dimethyl-3,4-dihydro-2H-chromen-4-yl]-N′-(1-methyl-1H-indazol-4-yl)urea;
N-[(4R)-6,8-difluoro-2,2-dimethyl-3,4-dihydro-2H-chromen-4-yl]-N′-isoquinolin-5-ylurea;
N-[(4R)-6,8-difluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-6,8-difluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-8-fluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-8-fluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-8-fluoro-2,2-dimethyl-3,4-dihydro-2H-chromen-4-yl]-N′-isoquinolin-5-ylurea;
N-[(4R)-7-fluoro-2,2-dimethyl-3,4-dihydro-2H-chromen-4-yl]-N′-isoquinolin-5-ylurea;
N-[(4R)-7-fluoro-2,2-dimethyl-3,4-dihydro-2H-chromen-4-yl]-N′-(-methyl-1H-indazol-4-yl)urea;
N-[(4R)-8-fluoro-2,2-dimethyl-3,4-dihydro-2H-chromen-4-yl]-N′-(-methyl-1H-indazol-4-yl)urea;
N-[(4R)-2,2-diethyl-6-fluoro-3,4-dihydro-2H-chromen-4-yl]-N′-(1-methyl-1H-indazol-4-yl)urea;
N-[(4R)-2,2-dimethyl-3,4-dihydro-2H-chromen-4-yl]-N′-isoquinolin-5-ylurea;
N-[(4R)-2,2-diethyl-6-fluoro-3,4-dihydro-2H-chromen-4-yl]-N′-isoquinolin-5-ylurea;
N-[(4R)-7-fluoro-2,2-dimethyl-3,4-dihydro-2H-chromen-4-yl]-N′-1H-indazol-4-ylurea;
N-[(4R)-7-fluoro-2,2-dimethyl-3,4-dihydro-2H-chromen-4-yl]-N′-[(7R)-7-hydroxy-5,6,7,8-tetrahydronaphthalen-1-yl]urea;
N-[(4R)-7-fluoro-2,2-dimethyl-3,4-dihydro-2H-chromen-4-yl]-N′-[(7S)-7-hydroxy-5,6,7,8-tetrahydronaphthalen-1-yl]urea;
N-[(4R)-8-fluoro-2,2-dimethyl-3,4-dihydro-2H-chromen-4-yl]-N′-1H-indazol-4-ylurea;
N-[(4R)-2,2-dimethyl-7-(trifluoromethyl)-3,4-dihydro-2H-chromen-4-yl]-N′-(methyl-1H-indazol-4-yl)urea;
N-[(4R)-2,2-dimethyl-7-(trifluoromethyl)-3,4-dihydro-2H-chromen-4-yl]-N′-isoquinolin-5-ylurea;
N-[(4R)-6-fluoro-2,2-dimethyl-3,4-dihydro-2H-chromen-4-yl]-N′-(3-methylisoquinolin-5-yl)urea;
N-[(4R)-2,2-dimethyl-3,4-dihydro-2H-chromen-4-yl]-N′-[(7R)-7-hydroxy-5,6,7,8-tetrahydronaphthalen-1-yl]urea;
N-[(4R)-2,2-dimethyl-3,4-dihydro-2H-chromen-4-yl]-N′-[(7S)-7-hydroxy-5,6,7,8-tetrahydronaphthalen-1-yl]urea;
N-[(4R)-6-fluoro-2,2-dimethyl-3,4-dihydro-2H-chromen-4-yl]-N′-isoquinolin-8-ylurea;
N-[(4R)-2,2-dimethyl-7-(trifluoromethoxy)-3,4-dihydro-2H-chromen-4-yl]-N′-(1-methyl-1H-indazol-4-yl)urea;
N-[(4R)-2,2-dimethyl-7-(trifluoromethoxy)-3,4-dihydro-2H-chromen-4-yl]-N′-isoquinolin-5-ylurea;
N-[(4R)-2,2-dimethyl-7-(trifluoromethyl)-3,4-dihydro-2H-chromen-4-yl]-N′- H-indazol-4-ylurea;
N-[(4R)-2,2-dimethyl-8-(trifluoromethyl)-3,4-dihydro-2H-chromen-4-yl]-N′-(methyl-1H-indazol-4-yl)urea;
N-[(4R)-2,2-dimethyl-7-(trifluoromethyl)-3,4-dihydro-2H-chromen-4-yl]-N′-isoquinolin-8-ylurea;
N-[(4R)-2,2-dimethyl-8-(trifluoromethyl)-3,4-dihydro-2H-chromen-4-yl]-N′-isoquinolin-5-ylurea;
N-[(4R)-2,2-dimethyl-8-(trifluoromethyl)-3,4-dihydro-2H-chromen-4-yl]-N′-H-indazol-4-ylurea;
N-[(4R)-2,2-diethyl-7-fluoro-3,4-dihydro-2H-chromen-4-yl]-N′-(1-methyl-1H-indazol-4-yl)urea;
N-[(4R)-2,2-dimethyl-8-(trifluoromethyl)-3,4-dihydro-2H-chromen-4-yl]-N′-[(7R)-7-hydroxy-5,6,7,8-tetrahydronaphthalen-1-yl]urea;
N-[(4R)-2,2-diethyl-7-fluoro-3,4-dihydro-2H-chromen-4-yl]-N′-isoquinolin-5-ylurea;
N-[(4R)-2,2-diethyl-7-(trifluoromethyl)-3,4-dihydro-2H-chromen-4-yl]-N′-(methyl-1H-indazol-4-yl)urea;
N-[(4R)-2,2-diethyl-8-fluoro-3,4-dihydro-2H-chromen-4-yl]-N′-(1-methyl-1H-indazol-4-yl)urea;
N-[(4R)-2,2-diethyl-7-(trifluoromethyl)-3,4-dihydro-2H-chromen-4-yl]-N′-isoquinolin-5-ylurea;
N-[(4R)-2,2-diethyl-8-fluoro-3,4-dihydro-2H-chromen-4-yl]-N′-[(7R)-7-hydroxy-5,6,7,8-tetrahydronaphthalen-1-yl]urea;
N-[(4R)-2,2-dimethyl-7-(trifluoromethyl)-3,4-dihydro-2H-chromen-4-yl]-N′-[(7R)-7-hydroxy-5,6,7,8-tetrahydronaphthalen-1-yl]urea;
N-[(4R)-2,2-diethyl-6-fluoro-3,4-dihydro-2H-chromen-4-yl]-N′-[(7R)-7-hydroxy-5,6,7,8-tetrahydronaphthalen-1-yl]urea;
N-[(4R)-2,2-diethyl-8-(trifluoromethoxy)-3,4-dihydro-2H-chromen-4-yl]-N′-(methyl-1H-indazol-4-yl)urea;
N-[(4R)-2,2-diethyl-6-fluoro-3,4-dihydro-2H-chromen-4-yl]-N′-(3-methylisoquinolin-5-yl)urea;
N-[(4R)-2,2-diethyl-8-fluoro-3,4-dihydro-2H-chromen-4-yl]-N′-isoquinolin-5-ylurea;
N-[(4R)-2,2-diethyl-6-fluoro-3,4-dihydro-2H-chromen-4-yl]-N′-1H-indazol-4-ylurea;
N-(1-methyl-1H-indazol-4-yl)-N′-[(4R)-8-(trifluoromethyl)-3,4-dihydro-2H-chromen-4-yl]urea;
N-[(4R)-2,2-diethyl-6,8-difluoro-3,4-dihydro-2H-chromen-4-yl]-N′-(1-methyl-1H-indazol-4-yl)urea;
N-[(4R)-6-fluoro-2,2-dipropyl-3,4-dihydro-2H-chromen-4-yl]-N′-(1-methyl-1H-indazol-4-yl)urea;
N-[(4R)-2,2-diethyl-8-fluoro-3,4-dihydro-2H-chromen-4-yl]-N′-(3-methylisoquinolin-5-yl)urea;
N-1H-indazol-4-yl-N′-[(4R)-8-(trifluoromethyl)-3,4-dihydro-2H-chromen-4-yl]urea;
N-isoquinolin-5-yl-N′-[(4R)-8-(trifluoromethyl)-3,4-dihydro-2H-chromen-4-yl]urea and 5N-[(4R)-8-fluoro-2,2-dipropyl-3,4-dihydro-2H-chromen-4-yl]-N′-(1-methyl-1H-indazol-4-yl)urea;
N-[(4R)-6-fluoro-2,2-bis(fluoromethyl)-3,4-dihydro-2H-chromen-4-yl]-N′-(3-methylisoquinolin-5-yl)urea;
N-[(4R)-8-fluoro-2,2-dipropyl-3,4-dihydro-2H-chromen-4-yl]-N′-(3-methylisoquinolin-5-yl)urea;
N-[(4R)-7-chloro-2,2-diethyl-3,4-dihydro-2H-chromen-4-yl]-N′-(3-methylisoquinolin-5-yl)urea;
N-1H-indazol-4-yl-N-[(4R)-8-(trifluoromethoxy)-3,4-dihydro-2H-chromen-4-25 yl]urea;
N-[(4R)-2,2-diethyl-7-fluoro-3,4-dihydro-2H-chromen-4-yl]-N′-[(7S)-7-hydroxy-5,6,7,8-tetrahydronaphthalen-1-yl]urea;
N-[(4R)-6-fluoro-2,2-dipropyl-3,4-dihydro-2H-chromen-4-yl]-N′-[(7R)-7-hydroxy-5,6,7,8-tetrahydronaphthalen-1-yl]urea;
N-[(4R)-2,2-diethyl-6-fluoro-3,4-dihydro-2H-chromen-4-yl]-N′-[(7S)-7-hydroxy-5,6,7,8-tetrahydronaphthalen-1-yl]urea;
N-[(4R)-2,2-diethyl-7-fluoro-3,4-dihydro-2H-chromen-4-yl]-N′-[(7R)-7-hydroxy-5,6,7,8-tetrahydronaphthalen-1-yl]urea;
N-[(4R)-7-chloro-2,2-diethyl-3,4-dihydro-2H-chromen-4-yl]-N′-[(7R)-7-hydroxy-5,6,7,8-tetrahydronaphthalen-1-yl]urea;
N-[(4R)-7-chloro-2,2-diethyl-3,4-dihydro-2H-chromen-4-yl]-N′-[(7S)-7-hydroxy-5,6,7,8-tetrahydronaphthalen-1-yl]urea;
(R)-1-(3-methylisoquinolin-5-yl)-3-[8-(trifluoromethoxy)chroman-4-yl]urea;
(R)-1-[6-fluoro-2,2-bis(fluoromethyl)chroman-4-yl]-3-(3-methylisoquinolin-5-yl)urea; and salts or hydrates or solvates thereof.

19. A pharmaceutical dosage form, comprising the solid dispersion product of claim 1.

20. A process for preparing a solid dispersion product comprising at least one pharmaceutically active agent, which process comprises
a) preparing a liquid mixture containing the at least one active agent, at least one pharmaceutically acceptable matrix-forming agent, at least one pharmaceutically acceptable surfactant and at least one solvent, and
b) removing the solvent(s) from the liquid mixture to obtain the solid dispersion product.

21. The process of claim 20, wherein the liquid mixture is prepared by dissolving the pharmaceutically acceptable matrix-forming agent to obtain a matrix-forming agent solution, and adding the active agent and the pharmaceutically acceptable surfactant to the solution.

22. The process of claim 20, wherein the liquid mixture has a dry matter content of up to 90% by weight.

23. The process of claim 20, wherein removing of the solvent is carried out by spray-drying, drum drying, belt drying, tray drying or combinations of two or more thereof.

24. The process of claim 20, wherein the solvent is selected from the group consisting of alkanols, hydrocarbons, halogenated hydrocarbons, ketons, esters, ethers and combinations of two or more thereof.

25. The process of claim 20, further comprising compressing the solid dispersion product to obtain a tablet.

26. The process of claim 25, wherein at least one additive selected from flow regulators, disintegrants, bulking agents and lubricants is added before compressing.

27. The process of claim 20, further comprising filling the solid dispersion product into capsules.

28. The process of claim 20, wherein at least one filler is added to the liquid mixture before removing the solvent(s).

29. The process of claim 20, wherein the mass ratio of active agent and pharmaceutically acceptable matrix-forming agent is from 0.01:1 to 1:3.

30. The process of claim 20, wherein the mass ratio of active agent and pharmaceutically acceptable surfactant is from 0.1:1 to 1:7.

31. The process of claim 20, wherein the pharmaceutically acceptable matrix-forming agent is selected from the group consisting of cyclodextrines, pharmaceutically acceptable polymers, lipids or combinations of two or more thereof.

32. The process of claim 20, wherein said pharmaceutically acceptable matrix-forming agent is selected from the group consisting of cellulose esters, cellulose ethers, cellulose ether-esters, maltodextrines, N-vinyl pyrrolidone homopolymers, N-vinyl pyrrolidone copolymers and combinations of two or more thereof.

33. The process of claim 20, wherein said pharmaceutically acceptable matrix-forming agent is selected from the group consisting of poly N-vinylpyrrolidones, copolymers of N-vinyl pyrrolidone and vinyl acetate and combinations thereof.

34. The process of claim 20, wherein the pharmaceutically acceptable surfactant is selected from the group consisting of polyol fatty acid esters, polyalkoxylated polyol fatty acid esters, polyalkoxylated fatty alcohol ethers, tocopheryl compounds or combinations of two or more thereof.

35. The process of claim 20, wherein the pharmaceutically acceptable surfactant comprises a combination of two or more pharmaceutically acceptable surfactants.

36. The process dispersion product of claim 20, wherein the pharmaceutically acceptable surfactant comprises at least one surfactant having an HLB value of 10 or more.

37. The process of claim 35 wherein the combination of pharmaceutically acceptable surfactants comprises (i) at least one tocopheryl compound having a polyalkylene glycol moiety and (ii) at least one polyalkoxylated polyol fatty acid ester.

38. The process of claim 37, wherein the tocopheryl compound is alpha tocopheryl polyethylene glycol succinate.

39. The process of claim 37, wherein the polyalkoxylated polyol fatty acid ester is a polyalkoxylated glyceride.

40. The process of claim 37, wherein the mass ratio of tocopheryl compound and polyalkoxylated polyol fatty acid ester is in the range of from 0.2:1 to 1:1.
